SEQUENCE LISTING (23 sequences)

SEQ ID NO: 1 - protein, 5 aa, synthetic construct
EHIPA

SEQ ID NO: 2 - protein, 5 aa, synthetic construct
GPRPK

SEQ ID NO: 3 - protein, 8 aa, synthetic construct
HAIYPHRH

SEQ ID NO: 4 - protein, 11 aa, synthetic construct
DLSLRDWGCLW

SEQ ID NO: 5 - protein, 11 aa, synthetic construct
DISLPRWGCLW

SEQ ID NO: 6 - protein, 6 aa, synthetic construct
GSRYRY

SEQ ID NO: 7 - protein, 6 aa, synthetic construct
RYYYAI

SEQ ID NO: 8 - protein, 6 aa, synthetic construct
AAHIYY

SEQ ID NO: 9 - protein, 6 aa, synthetic construct
IYRIGR

SEQ ID NO: 10 - protein, 6 aa, synthetic construct
HSKIYK

SEQ ID NO: 11 - protein, 6 aa, synthetic construct
ADRYGH

SEQ ID NO: 12 - protein, 6 aa, synthetic construct
DRIYYY

SEQ ID NO: 13 - protein, 6 aa, synthetic construct
DKQRII

SEQ ID NO: 14 - protein, 6 aa, synthetic construct
RYYDYG

SEQ ID NO: 15 - protein, 6 aa, synthetic construct
YRIDRY

SEQ ID NO: 16 - protein, 4 aa, synthetic construct
HYAI

SEQ ID NO: 17 - protein, 4 aa, synthetic construct
FRYY

SEQ ID NO: 18 - protein, 4 aa, synthetic construct
HRRY

SEQ ID NO: 19 - protein, 4 aa, synthetic construct
RYFF

SEQ ID NO: 20 - protein, 4 aa, synthetic construct
DKSI

SEQ ID NO: 21 - protein, 4 aa, synthetic construct
DRNI

SEQ ID NO: 22 - protein, 4 aa, synthetic construct
HYFD

SEQ ID NO: 23 - protein, 4 aa, synthetic construct
YRFD

(19) United States
(12) Patent Application Publication
(10) Pub. No.: US 2025/0215050 A1
(43) Pub. Date: Jul. 3, 2025

(54) COMPOSITIONS AND METHODS FOR PURIFYING BIOLOGICAL FLUIDS

(71) Applicant: North Carolina State University, Raleigh, NC (US)

(72) Inventors: Stefano Menegatti, Raleigh, NC (US); Sobhana Alekhya Sripada, Raleigh, NC (US); Wenning Chu, Raleigh, NC (US); Ruben G. Carbonell, Raleigh, NC (US); Rebecca Ashton Bradley Lavoie, Raleigh, NC (US)

(21) Appl. No.: 18/701,164

(22) PCT Filed: Oct. 14, 2022
(86) PCT No.: PCT/US22/78136

§ 371 Date: Apr. 12, 2024

Related U.S. Application Data
(60) Provisional application No. 63/256,278, filed on Oct. 15, 2021.

(51) Int. Cl. C07K 7/06; C07K 1/22; C07K 5/107; C07K 5/11; C07K 5/113; C07K 5/117

(52) U.S. Cl. CPC C07K 7/06; C07K 1/22; C07K 5/1016; C07K 5/1019; C07K 5/1021; C07K 5/1024

(57) ABSTRACT

The present disclosure provides materials and methods related to the purification of a biologic from a biological fluid. In particular, the present disclosure provides compositions, and related methods, comprising peptide ligands capable of removing process-related impurities (e.g., host cell proteins, nucleic acids, and media components) and product-related impurities (e.g., product fragments, product aggregates, and inactive forms derived from product degradation by or association with other species in the cell culture harvest) from biological fluids during the production of a biologic.

### CROSS REFERENCE TO RELATED APPLICATIONS

This application claims priority to and the benefit of U.S. Provisional Patent Application No. 63/256,278 filed Oct. 15, 2021, which is incorporated herein by reference in its entirety and for all purposes.

### SEQUENCE LISTING

The text of the computer readable sequence listing filed herewith, titled “NCSU-39568-601.xml”, created Oct. 14, 2022, having a file size of 20,808 bytes, is hereby incorporated by reference in its entirety.

### FIELD

The present disclosure provides materials and methods related to the purification of a biologic from a biological fluid. In particular, the present disclosure provides compositions, and related methods, comprising peptide ligands capable of removing process-related impurities (e.g., host cell proteins, nucleic acids, and media components) and product-related impurities (e.g., product fragments, product aggregates, and inactive forms derived from product degradation by or association with other species in the cell culture harvest) from biological fluids during the production of a biologic.

### BACKGROUND

The growing clinical application of therapeutic monoclonal antibodies (mAbs) and the introduction of their next-generation variants (e.g., engineered antibody fragments, antibody-drug conjugates (ADCs), and multi-specific antibodies) in the fight against cancer, metabolic, neurodegenerative disorders, autoimmune diseases as well as infectious diseases (e.g., COVID-19) calls for improved biomanufacturing strategies that increase productivity and throughput, while reducing cost and environmental impact of these processes. In this context, initiatives such as Industry 4.0 are seeking futuristic mAb manufacturing reliant on single-use technologies that can minimize process footprint and buffer usage, hence enabling continuous operation and faster process validation. These characteristics can accelerate product delivery to clinics, potentially shortening “bench-to-clinic” time for newer products, and optimizing the use of natural resources.

A major challenge in mAb manufacturing processes is the removal of process-related impurities, in particular the host cell proteins (HCPs), that are secreted together with the mAb product by recombinant expression systems, chiefly Chinese Hamster Ovary (CHO) cells that are most prevalently used. Due to their wide physicochemical and biomolecular diversity, their ability to associate with (co-elute) or degrade the mAb product, and strong immunogenic potential, HCPs must be carefully removed prior to drug product formulation. In current manufacturing processes, the burden of HCP removal is mostly sustained by the Protein A step, where the mAb is captured and concentrated while removing most of the HCPs present in CHO cell culture harvests. The growing application of proteomics in bioprocess monitoring, however, has documented that the Protein A capture step fails to remove several HCPs that pose a threat to patients' health due to their immunogenicity or ability to degrade the mAb product during storage. Additionally, some of these HCPs can escape capture by the subsequent steps of intermediate and final polishing, and have been reported to cause delays in FDA clinical trials and approval process, as well as recalls of mAb batches. Accomplishing the removal of these persistent, high risk (HR—) HCPs requires extensive optimization of post-capture chromatographic steps and causes a significant financial impact on downstream biomanufacturing.

More recently, viral vectors (VVs) have emerged as an additional family of tools in modern medicine and biotechnology owing to their role as delivery agents for gene therapies targeting rare diseases, oncolytic agents to fight aggressive forms of cancer, vaccine platforms to counter infectious diseases, and a gateway to engineer cell therapies as well as plants and animals for a sustainable agriculture. New VV designs are constantly being introduced with improved tissue targeting and gene delivery, and low genotoxicity, hepatotoxicity, and immunogenicity. While necessary for their success as next-generation biotherapeutics, the constant upgrading of the viral capsid and transgene design represents a challenge for large-scale manufacturing: (i) the biomolecular landscape of VV capsids is inherently complex—to date, 12 serotypes and over 100 variants of the adeno-associated virus (AAV) and >60 serotypes of adenovirus (Ad) have been isolated in human/nonhuman primate tissues; (ii) recombinant capsids selected via library screening for improved therapeutic efficacy and safety can present significant differences—in terms of composition, ratio, and arrangement of virion proteins-compared to the corresponding native serotype; and (iii) the expression of VVs-whether native or recombinant—by engineered cells is a highly defective process that returns a variety of product-related impurities including partial capsids and capsid fragments, capsid-bound DNA, and Rep-associated capsids with poor or no transduction activity. The effects of these complexity are well represented in the current landscape of commercial affinity adsorbents for VV purification. In the field of AAVs, ligands presented as pan-selective have been reported to show poor binding of some native serotypes and fail to bind several recombinant serotypes, leading to the development of serotype-specific ligands; furthermore, these ligands cannot distinguish between intact and broken or impurity-associated capsids; finally, the strong capsid:ligand association requires harsh elution, which can cause denaturation of the virion proteins and capsid aggregation, resulting in poor transduction activity, as well as lower the resin lifetime by preventing a full regeneration of the binding surface. The affinity available for the affinity purification of other VVs such as lentivirus (LV) and Ad are rather limited and suffer—together with the high cost—from the same limitations listed above for AAV-targeted adsorbents. Finally, no affinity adsorbent is available for a number of VVs—such as herpes virus, baculovirus, rabies virus, etc.—despite their relevance in in vivo or ex vivo gene and cell therapies. The rapidly evolving scenario of VV manufacturing suggests reconfiguring affinity purification technology in a product-agnostic direction. This is corroborated by the uniformity of VV expression systems, which—in sharp contrast with the diversity of VV products—are represented almost solely by HEK293 and Sf9 cells, while other cell lines, such as Vero and Per.C3, are seldom utilized.

This calls for tools enabling a flexible platform for VV purification that comprises steps operated in flow-through mode to capture process- and product-related impurities (i.e., host cell proteins (HCPs), host cell DNA (hcDNA), plasmid DNA (pDNA), as well as capsid fragments, capsid-bound DNA, and Rep-associated capsids, etc.) while allowing the product VV to flow-through un-bound. This represents a transformative approach to VV purification, in that it is (a) flexible, as it can be readily implemented to the purification of different VVs and, for a given VV family, interchangeably across its different serotypes; (b) robust, as it can easily address variations in process- and product-related impurities coming from the upstream segment with minimal optimization; (c) rapid, as the major step of impurity clearance is performed in flow-through mode, which is in itself fast, thus enabling subsequent bind-and-elute mode steps to operate on “simpler” downstream stream; (d) affords higher product quality and safety, by removing-right at the onset of the purification pipeline-impurities that are traditionally challenging to remove and pose a threat to patient safety and product stability.

### SUMMARY

Embodiments of the present disclosure include a composition for purifying a target biologic from a biological fluid. In accordance with these embodiments, the composition includes at least one peptide ligand that is at least four amino acids in length and comprises at least one basic amino acid and at least one hydrophilic amino acid.

In some embodiments, the at least one peptide ligand comprises a hydrophobic amino acid or a positively charged amino acid at the second amino acid position. In some embodiments, the at least one peptide ligand comprises a hydrophobic amino acid or a negatively charged amino acid at the fourth amino acid position. In some embodiments, the at least one peptide ligand comprises an acidic amino acid directly adjacent to a hydrophobic amino acid or a negatively charged amino acid. In some embodiments, the at least one peptide ligand comprises a polar amino acid directly adjacent to cationic amino acid or an aliphatic amino acid. In some embodiments, a negatively charged amino acid in the peptide ligand is: (i) not directly adjacent to a polar amino acid; (ii) directly adjacent to an aliphatic amino acid or an aromatic amino acid; and/or (iii) directly adjacent to a positively charged amino acid. In some embodiments, an aromatic amino acid in the peptide ligand is: (i) directly adjacent to an aliphatic amino acid; (ii) directly adjacent to an anionic amino acid; and/or (iii) directly adjacent to a cationic amino acid.

In some embodiments, the at least one peptide ligand binds at least one host cell protein (HCP), at least one high-risk HCP, at least one host cell nucleic acid, aggregates of the target biologic, and/or an impurity derived from the target biologic.

In some embodiments, the at least one peptide ligand exhibits a K_{D} from about 10⁻⁹ M to about 10⁻⁵ M for the HCP, the host cell nucleic acid, the aggregates of the target biologic, and/or the impurity derived from the target biologic.

In some embodiments, the at least one peptide ligand comprises a linker. In some embodiments, the linker is bound to the C-terminus of the peptide ligand, and wherein the linker comprises a Glyₙ or a [Gly-Ser-Gly]ₘ, wherein 6≥n≥1 and 3≥m≥1.

In some embodiments, the at least one peptide ligand is bound to a solid support. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate, polyolefin, polyester, polysaccharide, iron oxide, silica, titania, and/or zirconia.

In some embodiments, the at least one peptide ligand is no more than 15 amino acids in length.

In some embodiments, the biological fluid comprises a supernatant and/or a cellular lysate.

In some embodiments, the biological fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof.

In some embodiments, the biological fluid is derived from HEK293 cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof.

In some embodiments, the biological fluid is derived from yeast cells. In some embodiments, the yeast cells are selected from the group consisting of P. pastoris, S. cerevisiae, and S. boulardii, or any derivatives or variants thereof.

In some embodiments, the biological fluid is derived from a virus production cell line. In some embodiments, the virus production cell line is selected from the group consisting of MDCK-S, MDCK-A, Vero cells, LLC-MK2D, PER.C6, EB66, and AGE1.CR cells, or any derivatives or variants thereof.

In some embodiments, the target biologic is one or more of a protein, peptide or polypeptide; an oligonucleotide or a polynucleotide; a virus or a virus-like particle; an exosome or an extracellular vesicle; a cell or cell organelle; or a small molecule.

In some embodiments, the biological fluid comprises a pH from about 3.0 to about 9.0.

In some embodiments, the biological fluid comprises a conductivity of about 1 to about 50 mS/cm.

In some embodiments, the at least one peptide ligand is selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof.

In some embodiments, the at least one peptide ligand comprises at least two peptide ligands selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof.

In some embodiments, the at least one peptide ligand comprises at least three peptide ligands selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof.

In some embodiments, the at least one peptide ligand comprises at least four peptide ligands selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof.

In some embodiments, the composition comprises EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof.

In some embodiments, the composition further comprises at least one peptide ligand selected from the group consisting of: GSRYRY (SEQ ID NO: 6), RYYYAI (SEQ ID NO: 7), AAHIYY (SEQ ID NO: 8), IYRIGR (SEQ ID NO: 9), HSKIYK (SEQ ID NO: 10), ADRYGH (SEQ ID NO: 11), DRIYYY (SEQ ID NO: 12), DKQRII (SEQ ID NO: 13), RYYDYG (SEQ ID NO: 14), YRIDRY (SEQ ID NO: 15), HYAI (SEQ ID NO: 16), FRYY (SEQ ID NO: 17), HRRY (SEQ ID NO: 18), RYFF (SEQ ID NO: 19), DKSI (SEQ ID NO: 20), DRNI (SEQ ID NO: 21), HYFD (SEQ ID NO: 22), and YRFD (SEQ ID NO: 23), or any derivatives or variants thereof.

Embodiments of the present disclosure include an adsorbent comprising any of the compositions described herein.

Embodiments of the present disclosure also include a method of purifying a target biologic from a biological fluid. In accordance with these embodiments, the method includes contacting a composition comprising any of the at least one peptide ligands and/or adsorbents described herein with the biological fluid comprising the target biologic, and collecting the biological fluid in flow-through mode, wherein the biological fluid comprises the target biologic. In some embodiments, the at least one peptide ligand binds at least one host cell protein (HCP), at least one high-risk HCP, at least one host cell nucleic acid, aggregates of the target biologic, and/or an impurity derived from the target biologic in a retentate.

In some embodiments, the method further comprises performing affinity chromatography on the biological fluid comprising the target biologic.

In some embodiments, the biological fluid comprises a pH from about 3.0 to about 9.0. In some embodiments, the biological fluid comprises a conductivity of about 1 to about 50 mS/cm.

In some embodiments, the method is performed under static binding conditions. In some embodiments, the method is performed under dynamic binding conditions.

### BRIEF DESCRIPTION OF THE DRAWINGS

FIG. 1: Mechanism of “flow-through affinity chromatography,” wherein an ensemble of synthetic ligands captures the spectrum of HCPs present in a cell culture harvest without retaining the target product (e.g., a therapeutic antibody or a virus).

FIGS. 2A-2B: Static binding studies-reported as Langmuir isotherm binding data-obtained in (FIG. 2A) non-competitive conditions by incubating solutions of CHO HCPs with (▪) G.1 or (♦) G.2 LigaGuard™ resins, or NIST mAb at different concentrations with (□) G.1 or (⋄) G.2 LigaGuard™ resin; (FIG. 2B) competitive conditions detailing HCP binding of G.2 LigaGuard™ with 1 mg/mL (●) and 5 mg/mL (▴) NISTmAb, and G.1 LigaGuard™ with 1 mg/mL (∘) and 5 mg/mL (Δ) NISTmAb, respectively.

FIG. 3: Contour maps correlating the values of mAb yield and purity as functions of load volume (CVs) obtained by loading industrial HCCFs on G.1 and G.2 LigaGuard™ resins (residence time of 1 min).

FIG. 4: Compared values of cumulative yield obtained under optimal loading of industrial HCCFs on G.1 and G.2 LigaGuard™ resins (residence time of 1 min).

FIG. 5: Box and Whisker plots of HMW CHO and LMW CHO content in the industrial HCCFs and the corresponding effluents obtained with G.1 and G.2 LigaGuard™ resins (residence time of 1 min).

FIG. 6: Cumulative HCP LRVs (HCP cLRVs) obtained by loading industrial CHO HCCFs containing therapeutic mAbs on G.1 and G.2 LigaGuard™ resins at the residence time (RT) of 1 min or 2 min (□: G.1-RT: 1 min; ▪: G.2-RT: 1 min; ∘: G.1-RT: 2 min; ●: G.2-RT: 2 min). The corresponding values of fractional LRVs are reported in FIG. 11.

FIG. 7: Fraction of HCPs captured at different values of load volumes (CVs) by G.1 and G.2 LigaGuard™ resins, and therefore absent in the effluent streams, expressed as % values of the total number of HCPs in the corresponding harvests.

FIGS. 8A-8B: Protein adsorption kinetic curves, reported as fraction of the amount of protein bound per volume of resin (q) vs. maximum binding capacity at the equilibrium (Qₘₐₓ), obtained by incubating a null CHO-S HCCF at 0.7 mg HCP per mL with (⋄) G.1 or (♦) G.2 LigaGuard™ resin or a solution pure NIST mAb at 0.8 mg/mL in PBS at pH 7.4 with (∘) G.1 or (●) G.2 LigaGuard™ resin (FIG. 8A). Breakthrough curves obtained by injecting a CHO HCCF featuring a mAb titer of 1.38 mg/mL and an HCP titer of 0.46 mg/mL on G.2 LigaGuard™ resins at the residence time of (▪) 0.5 min, (♦) 1 min, (▴) 2 min, and (●) 5 min (FIG. 8B).

FIG. 9: Profiles of mAb cumulative yield and purity and residual content of (%) HWM and LMW HCPs in the effluents obtained by loading different industrial HCCFs on G.1 LigaGuard™ resin (lines 1 and 2) and G.2 LigaGuard™ resin.

FIG. 10: Bubble plots of CHO HCPs in the industrial HCCFs utilized in this study presented by sequence-based values of isoelectric point (pI) and grand average of hydropathy (GRAVY); every point represents a unique HCP identified in the source HCCF by abundance (diameter of the dot) and sequence-based value of molecular weight (color).

FIG. 11: Fractional values of HCP LRVs (HCP fLRVs) obtained by loading industrial CHO HCCFs containing therapeutic mAbs on G.1 and G.2 LigaGuard™ resins at the residence time (RT) of 1 min or 2 min (□ G.1-RT: 1 min; ∘: G.1-RT: 2 min; ▪: G.2-RT: 1 min; ●: G.2-RT: 2 min).

FIG. 12: Distribution of CHO HCPs based on the values of sequence-based molecular weight in the pooled effluent obtained by loading industrial HCCFs on G.1 or G.2 LigaGuard™ resins at the residence time of 1 min.

FIG. 13: Distribution of CHO HCPs based on the values of sequence-based isoelectric point (pI) in the pooled effluent obtained by loading industrial HCCFs on G.1 or G.2 LigaGuard™ resins at the residence time of 1 min.

FIG. 14: Scheme of a mAb purification process wherein the effluent from the G.2 LigaGuard™ resin is fed to an affinity adsorbent, either a Protein A-based Toyopearl AF-rProtein A-650F resin or LigaTrap® Human IgG resin packed in a 0.1 mL chromatography column.

FIGS. 15A-15B: Representative images of a reducing SDS-PAGE gel (Coomassie stained in FIG. 15A; silver stained in FIG. 15B) demonstrating the presence of mAb4 in various flow-through fractions (e.g., FT1-FT10) of cell culture fluid from CHO cells purified using the peptide ligands of the present disclosure.

FIGS. 16A-16B: Representative images of a reducing SDS-PAGE gel (Coomassie stained in FIG. 16A; silver stained in FIG. 16B) demonstrating the presence of mAb3 in various flow-through fractions (e.g., FT1-FT10) of cell culture fluid from CHO cells purified using the peptide ligands of the present disclosure.

FIGS. 17A-17B: Representative images of a reducing SDS-PAGE gel (Coomassie stained in FIG. 17A; silver stained in FIG. 17B) demonstrating the presence of mAb2 in various flow-through fractions (e.g., FT1-FT10) of cell culture fluid from CHO cells purified using the peptide ligands of the present disclosure.

FIGS. 18A-18B: Representative images of a reducing SDS-PAGE gel (Coomassie stained in FIG. 18A; silver stained in FIG. 18B) demonstrating the presence of GmAb3 in various flow-through fractions (e.g., FT1-FT10) of cell culture fluid from CHO cells purified using the peptide ligands of the present disclosure.

FIGS. 19A-19B: Representative images of a reducing SDS-PAGE gel (Coomassie stained in FIG. 19A; silver stained in FIG. 19B) demonstrating the presence of GmAb2 in various flow-through fractions (e.g., FT1-FT10) of cell culture fluid from CHO cells purified using the peptide ligands of the present disclosure.

FIGS. 20A-20C: Values of (FIG. 20A) pIgG adsorption (mg of pIgG per mL of LigaTrap™ resin), (FIG. 20B) elution yield (%), and (FIG. 20C) pIgG purity in the eluted fraction (%) obtained by purifying pIgG from an Ig-rich paste using LigaTrap™ resin and binding buffers with different pH (6.5, 7.0, 7.4, and 8.0) and NaCl concentrations (0, 0.15, 0.25, and 0.5 M).

FIGS. 21A-21D: SDS-PAGE analysis (reducing conditions, Coomassie staining) of the chromatographic fractions obtained by purifying pIgG from an Ig-rich paste using LigaTrap™ resin and binding buffers with different pH: (FIG. 21A) 6.5, (FIG. 21B) 7.0, (FIG. 21C) 7.4, and (FIG. 21D) 8.0, and sodium chloride concentrations (0, 0.15, 0.25, and 0.5 M). Labels: MW, molecular weight marker; hIgG, human polyclonal IgG standard; F, feedstock obtained by dissolving the Ig-rich paste in binding buffer; FT-X, flow-through fraction obtained by loading Ig-rich paste diluted in a binding buffer with XM sodium chloride concentration; E-X, elution fraction obtained by loading Ig-rich paste diluted in a binding buffer with XM sodium chloride concentration; HSA, human serum albumin; IgG HC, heavy chain of IgG; IgG LC, light chain of IgG.

FIGS. 22A-22C: Values of (FIG. 22A) pIgG adsorption (mg of pIgG per mL of LigaTrap™ resin), (FIG. 22B) elution yield (%), and (FIG. 22C) pIgG purity in the eluted fraction (%) obtained by purifying pIgG from an Ig-rich paste using LigaTrap™ resin and binding buffers with different pH (7.4 and 8.0) and sodium caprylate concentrations (0, 25, 50 and 75 mM), and constant NaCl concentration (0.5 M).

FIGS. 23A-23B: SDS-PAGE analysis (reducing conditions, Coomassie staining) of the chromatographic fractions obtained by purifying pIgG from an Ig-rich paste using LigaTrap™ resin and binding buffers with pH of either (FIG. 23A) 7.4 or (FIG. 23B) 8.0, and sodium caprylate concentrations (0, 25, 50 and 75 mM), and constant NaCl concentration (0.5 M). Labels: MW, molecular weight marker; hIgG, human polyclonal IgG standard; F, feedstock obtained by dissolving the Ig-rich paste in binding buffer; FT-X, flow-through fraction obtained by loading Ig-rich paste diluted in a binding buffer with XM sodium caprylate concentration; E-X, elution fraction obtained by loading Ig-rich paste diluted in a binding buffer with XM sodium caprylate concentration; HSA, human serum albumin; IgG HC, heavy chain of IgG; IgG LC, light chain of IgG.

FIGS. 24A-24E: Profiles of pIgG flow-through yield (Y_{pIgG}) vs. loading volume obtained by injecting a solution of pIgG at 3 mg/mL in different binding buffers: (FIG. 24A) 20 mM piperazine HCl buffer at pH 5.0 and 5.5; 20 mM Bis-Tris HCl buffer at pH5.5; (FIG. 24B) 20 mM citric acid and Na₂HPO₄, KH₂PO₄ and Na₂HPO₄, and Bis-Tris HCl buffer at pH 6.0; (FIG. 24C) 20 mM citric acid and Na₂HPO₄, KH₂PO₄ and Na₂HPO₄, and Bis-Tris HCl buffer at pH 6.5; (FIG. 24D) 20 mM citric acid and Na₂HPO₄, KH₂PO₄ and Na₂HPO₄, Bis-Tris HCl, and Tris HCl buffer at pH 7.0; and (FIG. 24E) 20 mM citric acid and Na₂HPO₄, KH₂PO₄ and Na₂HPO₄, Tris HCl buffer at pH 7.4, and PBS buffer at pH 7.4—on first-generation LigaGuard™ resin.

FIGS. 25A-25C: Profiles of non-Ig plasma protein capture (Q_{PP}) vs. loading volume obtained by injecting diluted Ig-depleted plasma at 6 mg/mL in different binding buffers—namely, 20 mM piperazine HCl buffer at pH 5.0 and 5.5 (FIG. 25C); 20 mM Bis-Tris HCl buffer at pH 5.5 and 6.0 (FIG. 25B); and PBS buffer at pH 7.4 (FIG. 25A)—on LigaGuard™ resin.

FIGS. 26A-26D: Profiles of (FIG. 26A) pIgG flow-through yield (Y_{pIgG}), (FIG. 26B) non-Ig plasma protein capture (Q_{PP}), (FIG. 26C) ratio of pIgG titer in the effluent vs. feedstock (C_{pIgG}/C_{pIgG}*), and (FIG. 26D) ratio of non-Ig plasma protein titer in the effluent vs. feedstock (C_{PP}/C_{PP}*) vs. loading volume obtained by injecting plasma diluted using 20 mM Bis-Tris HCl buffer at pH 6.0 to a total protein titer of either 10 mg/mL (10-fold dilution, D10X) or 5.1 mg/mL (20-fold dilution, D20X) on first-generation LigaGuard™ resin.

FIG. 27: SDS-PAGE analysis (reducing conditions, Coomassie staining) of the chromatographic fractions obtained by purifying pIgG from diluted cryo-rich plasma using LigaGuard™ resin and 20 mM Bis-Tris HCl buffer at pH 6.0 as binding buffer. Labels: Feed D10X, 10-fold diluted plasma; FT D10X 2 CV, flow-through fraction obtained by loading 1 mL of 10-fold diluted plasma; FT D10X 6 CV, flow-through fraction obtained by loading 3 mL of 10-fold diluted plasma; FT D10X 10 CV, flow-through fraction obtained by loading 5 mL of 10-fold diluted plasma (i.e., cut-off value of loading volume); FT D10X Chased, flow-through fraction obtained by chasing the loading of 10-fold diluted plasma with the corresponding binding buffer; FT D20X 2 CV, flow-through fraction obtained by loading 1 mL of 20-fold diluted plasma; FT D20X 6 CV, flow-through fraction obtained by loading 3 mL of 20-fold diluted plasma; FT D20X 10 CV, flow-through fraction obtained by loading 5 mL of 20-fold diluted plasma (i.e., cut-off value of loading volume); FT D20X Chased, flow-through fraction obtained by chasing the loading of 20-fold diluted plasma with the corresponding binding buffer.

FIGS. 28A-28G: Profiles of (FIG. 28A) pIgG flow-through yield (Y_{pIgG}) and (FIG. 28B) non-Ig plasma protein capture (Q_{PP}) vs. loading volume obtained by respectively injecting a solution of pIgG at 3.0 mg/mL and Ig-depleted plasma at 5.6 mg/mL in 20 mM Bis-Tris HCl buffer at pH 6.0 on second-generation LigaGuard™ resin; (FIG. 28C) pIgG flow-through yield (Y_{pIgG}) and (FIG. 28D) purity (P_{pIgG}) vs. loading volume, and (FIG. 28E) Q_{PP} vs. loading volume obtained by injecting cryo-rich plasma diluted using 20 mM Bis-Tris HCl buffer at pH 6.0 to a total protein titer of either 15 mg/mL (5-fold dilution, D5X), 7.0 mg/mL (10-fold dilution, D10X), or 3.9 mg/mL (20-fold dilution, D20X) on second-generation LigaGuard™ resin, (FIG. 28F) pIgG flow-through yield (Y_{pIgG}) and (FIG. 28G) purity (P_{pIgG}) vs. loading volume by injecting cryo-poor plasma diluted using 20 mM Bis-Tris HCl buffer at pH 6.0 to a total protein titer of 6.4 mg/mL (10-fold dilution, D10X).

FIGS. 29A-29D: SDS-PAGE analysis (native conditions, Coomassie staining) of the chromatographic fractions obtained by purifying pIgG from cryo-rich plasma diluted using 20 mM Bis-Tris HCl buffer at pH 6.0 to a total protein titer of (FIG. 29A) 15 mg/mL (5-fold dilution, D5X), (FIG. 29B) 7.0 mg/mL (10-fold dilution, D10X), (FIG. 29C) 3.9 mg/mL (20-fold dilution, D20X), or (FIG. 29D) cryo-poor plasma diluted using 20 mM Bis-Tris HCl buffer at pH 6.0 to a total protein titer of 6.04 mg/mL (10-fold dilution, D10X). Labels: MW, molecular weight latter; hIgG, human polyclonal IgG standard; F, diluted plasma feedstock; and FTi, i-th flow-through fraction.

FIGS. 30A-30B: Two-column process comprising the second-generation LigaGuard™ adsorbent operated in flow-through mode followed by a LigaTrap™ adsorbent operated in bind-and-elute mode, and corresponding process (i.e., loading, composition of the buffers, and residence time) and performance (Y_{pIgG} and P_{pIgG}) parameters (FIG. 30A). SDS-PAGE analysis (reducing conditions, Coomassie staining) of the chromatographic fractions obtained by purifying pIgG from cryo-rich plasma diluted 10-fold using 20 mM Bis-Tris HCl buffer at pH 6.0 via the two-column process (FIG. 30B). Labels: MW, molecular weight latter; hIgG, human polyclonal IgG standard; F, diluted plasma feedstock; FT-LG, flow-through fraction from the LigaGuard™ adsorbent; FT-LT, flow-through fraction from the LigaTrap™ adsorbent; and E-LT, elution fraction from the LigaTrap™ adsorbent.

FIGS. 31A-31B: Two-column process comprising the second-generation LigaTrap™ adsorbent operated in bind-and-elute mode followed by a LigaGuard™ adsorbent operated in flow-through mode, and corresponding process (i.e., loading, composition of the buffers, and residence time) and performance (Y_{pIgG} and P_{pIgG}) parameters (FIG. 31A). SDS-PAGE analysis (reducing conditions, Coomassie staining) of the chromatographic fractions obtained by purifying pIgG from cryo-rich plasma diluted 10-fold using 20 mM Bis-Tris HCl buffer at pH 6.0 via the two-column process (FIG. 31B). Labels: MW, molecular weight latter; hIgG, human polyclonal IgG standard; HSA, human 6 serum albumin; F, diluted plasma feedstock; FT+W-LT, flow-through and wash fraction from the LigaTrap™ adsorbent; FT-E, elution fraction from the LigaTrap™ adsorbent; FT-CIP, clean in place fraction from the LigaTrap™ adsorbent; and FT n-LG (n=1, 2 and 3), flow-through fraction n from the LigaGuard™ adsorbent.

FIG. 32: DBC_{10%} (mg/mL resin) of IgG on LigaTrap™ Human IgG resin at different resident time (2 and 5 min) and initial concentration (5 and 10 mg/mL).

FIGS. 33A-33B: Binding capacity (Q, mg/mL), yield (Y, %) and purity (P, %) of polyclonal IgG purification from human cryo-rich plasma using (FIG. 33A) LigaTrap Human IgG resin and (FIG. 33B) Protein G Sepharose® Fast Flow at different IgG loading amount (10, 15, 20, 30, 40 and 50 mg/mL resin).

FIGS. 34A-34B: SDS-PAGE analysis (reducing conditions, Coomassie staining) of the chromatographic fractions obtained from IgG purification from human cryo-rich plasma on LigaTrap Human IgG resin (FIG. 34A) and Protein G Sepharose® Fast Flow (FIG. 34B) at different IgG loading amount (10, 15, 20, 30, 40 and 50 mg/mL resin). Labels: Feed, diluted human cryo-rich plasma; FT-n mg/mL (n=10, 15, 20, 30, 40 and 50), flow-through fraction at IgG loading amount of n mg/mL resin (n=10, 15, 20, 30, 40 and 50); E-n mg/mL (n=10, 15, 20, 30, 40 and 50), elution fraction at IgG loading amount of n mg/mL resin (n=10, 15, 20, 30, 40 and 75).

FIGS. 35A-35D: Comparison of the values of sequence-based isoelectric point (pI) and GRand A Verage of hYdropathicity (GRAVY) index of (FIG. 35A) CHO host cell proteins vs. (FIG. 35B) human plasma proteins; comparison of the values of sequence-based polarity (Zimmerman scale) and GRAVY index of (FIG. 35C) CHO host cell proteins vs. (FIG. 35D) human plasma proteins.

FIGS. 36A-36E: Profiles of pIgG flow-through ratio vs. loading volume obtained by injecting a solution of pIgG at 3 mg/mL in different binding buffers—namely, (FIG. 36A) 20 mM piperazine HCl buffer at pH 5.0 and 5.5; (FIG. 36B) 20 mM Bis-Tris HCl buffer at pH 5.5, 6.0, 6.5, and 7.0; (FIG. 36C) 20 mM citric acid and Na₂HPO₄ at pH 6.0, 6.5, 7.0, and 7.4; (FIG. 36D) 20 mM KH₂PO₄ and Na₂HPO₄ at pH 6.0, 6.5, 7.0, (FIG. 36E) and 7.4; 20 mM Tris HCl buffer at pH 7.0 and 7.4, and PBS buffer at pH 7.4—on LigaGuard™ resin.

FIGS. 37A-37E: Profiles of pIgG binding (Q_{pIgG}) vs. loading volume obtained by injecting a solution of pIgG at 3 mg/mL in different binding buffers—namely, (FIG. 37A) 20 mM piperazine HCl buffer at pH 5.0 and 5.5; (FIG. 37B) 20 mM Bis-Tris HCl buffer at pH 5.5, 6.0, 6.5, and 7.0; (FIG. 37C) 20 mM citric acid and Na₂HPO₄ at pH 6.0, 6.5, 7.0, and 7.4; (FIG. 37D) 20 mM KH₂PO₄ and Na₂HPO₄ at pH 6.0, 6.5, 7.0, (FIG. 37E) and 7.4; 20 mM Tris HCl buffer at pH 7.0 and 7.4, and PBS buffer at pH 7.4—on LigaGuard™ resin.

FIGS. 38A-38F: Profiles of pIgG (FIG. 38A) breakthrough ratio (C_{pIgG}/C_{pIgG}*) and (FIG. 38B) capture (Q_{pIgG}) vs. loading volume obtained by injecting a solution of pIgG at 3.0 mg/ml in 20 mM Bis-Tris HCl buffer at pH 6.0 on second-generation LigaGuard™ resin; (FIG. 38C) breakthrough ratio (C_{pIgG}/C_{pIgG}*) and (FIG. 38D) capture (Q_{pIgG}) vs. loading volume obtained by injecting cryo-rich plasma diluted using 20 mM Bis-Tris HCl buffer at pH 6.0 to a total protein titer of either 15 mg/mL (5-fold dilution, D5X), 7.0 mg/mL (10-fold dilution, D10X), or 3.9 mg/mL (20-fold dilution, D20X) on second-generation LigaGuard™ resin; (FIG. 38D) breakthrough ratio (C_{pIgG}/C_{pIgG}*) and (FIG. 38E) capture (Q_{pIgG}) vs. loading volume obtained by injecting cryo-poor plasma diluted using 20 mM Bis-Tris HCl buffer at pH 6.0 to a total protein titer of 6.4 mg/mL (10-fold dilution, D10X).

FIGS. 39A-39F: Profiles of non-Ig plasma proteins (FIG. 39A) breakthrough ratio (C_{PP}/C_{PP}*) and (FIG. 39B) capture (Q_{PP}) vs. loading volume obtained by injecting a solution of pIgG at 3.0 mg/ml in 20 mM Bis-Tris HCl buffer at pH 6.0 on second-generation LigaGuard™ resin; (FIG. 39C) breakthrough ratio (C_{PP}/C_{PP}*) and (FIG. 39D) capture (Q_{PP}) vs. loading volume obtained by injecting cryo-rich plasma diluted using 20 mM Bis-Tris HCl buffer at pH 6.0 to a total protein titer of either 15 mg/mL (5-fold dilution, D5X), 7.0 mg/mL (10-fold dilution, D10X), or 3.9 mg/mL (20-fold dilution, D20X) on second-generation LigaGuard™ resin; (FIG. 39D) breakthrough ratio (C_{PP}/C_{PP}*) and (FIG. 39E) capture (Q_{PP}) vs. loading volume obtained by injecting cryo-poor plasma diluted using 20 mM Bis-Tris HCl buffer at pH 6.0 to a total protein titer of 6.4 mg/mL (10-fold dilution, D10X).

FIGS. 40A-40C: (A) Schematic representation of capture of host cell protein impurities (HCP impurities) in flow-through mode from a clarified lysate of HEK293 cells expressing a therapeutic AAV using LigaGuard™ resin, resulting in purified AAVs in the effluent; (B) values of concentration of HEK293 host cell proteins in the flow-through effluent produced by feeding a clarified lysate of HEK293 cells expressing a therapeutic AAV2 through a LigaGuard™ resin (C_{FT} (mg/mL); black ▪); and corresponding values of HCP binding by the LigaGuard™ resin (Q (mg HCP per mL resin); red ●) vs. volume of clarified lysate loaded onto the LigaGuard™ resin (mL); (C) Table collating the values of host cell protein titer in the clarified lysate of HEK293 cells (Load), dynamic binding capacity at 10% breakthrough (DBC_{10%}) measured at different values of residence time (0.5 min, 1 min, 2 min, and 5 min).

FIGS. 41A-41D: Logarithmic removal values of host cell proteins (HCP LRV) obtained by loading clarified harvested cell culture fluids (HCCF) produced by (A) Chinese Hamster Ovary (CHO-K1) cells, (B, C) Human Embryonic Kidney (HEK293T) cells, or (D) HEK293FT cells onto LigaGuard™ resin vs. volume of clarified lysate loaded (expressed in column volumes, CVs).

FIG. 42: Values (%) of the ratios of number of host cell proteins (HCPs) identified in the #-th flow-through fraction (FT #) vs. number of host cell proteins (HCPs) identified in the feed obtained at incremental values of volume (20 CVs, 40 CVs, 60 CVs, and 80 CVs) of clarified CHO-K1, HEK293T, and HEK293FT harvested cell culture fluid (HCCF) loaded on LigaGuard™ resin.

FIG. 43: Schematic representation of capture of host cell protein impurities (HCP impurities) in flow-through mode from a clarified lysate of HEK293 cells expressing a therapeutic AAV using LigaGuard™ resin, resulting in purified AAVs in the effluent; average values of (i) recovery of AAV particles, (ii) recovery of encapsidated genome; (iii) logarithmic removal value of host cell DNA (DNA LRV); and (iv) logarithmic removal value of host cell proteins (HCP LRV) obtained by loading clarified lysates of HEK293 cells expressing therapeutic AAVs onto LigaGuard™.

FIGS. 44A-44C: Representative data using LigaGuard™ resin to purify AAV8 from HEK293 lysate in flow-through mode as shown with (A) affinity chromatography, (B) SEC-UPLC analysis, and (C) via ELISA to assess the recovery of AAV8.

FIG. 45: Representative data using LigaGuard™ resin to purify AAV2 in flow-through mode as shown with affinity chromatography from HEK293 HCPs.

FIGS. 46A-46B: Representative data using LigaGuard™ resin to purify AAV2 from HEK293 lysate in flow-through mode as shown with (A) affinity chromatography and (B) SEC-UPLC analysis followed by ELISA to assess the recovery of AAV2.

FIGS. 47A-47B: Representative data using LigaGuard™ resin to purify AAV6 from HEK293 lysate in flow-through mode as shown with (A) affinity chromatography and (B) SEC-UPLC analysis followed by ELISA to assess the recovery of AAV6.

FIGS. 48A-48B: Representative data using LigaGuard™ resin to purify AAV9 from HEK293 lysate in flow-through mode as shown with (A) affinity chromatography and (B) SEC-UPLC analysis followed by ELISA to assess the recovery of AAV9.

FIGS. 49A-49B: Representative data using LigaGuard™ resin to purify AAV8 from HEK293 lysate in flow-through mode as shown with (A) affinity chromatography and (B) SEC-UPLC analysis followed by ELISA to assess the recovery of AAV8.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “sequence identity” generally refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “target” or “target biologic” generally refers to a target protein, peptide, polypeptide, nucleic acid, ribonucleoprotein complex, nucleic acid construct, supramolecular construct, virus, viral construct, virus-like particle, cell, organelle, small molecule, and any combinations thereof, which may be present in a sample (e.g., biological fluid) comprising one or more process-related impurities and/or product-related substances. In some embodiments, the target or target biologic is an antibody or any antigen binding fragment/derivative thereof (e.g., monoclonal or polyclonal antibody). In other embodiments, the target or target biologic is a viral vector (e.g., AAV).

As used herein, the term “host cell protein” or “HCP” refers to any protein produced or encoded by the organism used to produce a recombinant polypeptide product and unrelated to the intended product. HCPs are generally undesirable in the final drug substance.

As used herein, a “mixture” comprises a target biologic of interest (for which purification is desired) and one or more contaminant or impurity. In some embodiments, the mixture is produced from a host cell or organism that expresses the protein of interest (either naturally or recombinantly). Such mixtures include, for example, cell cultures, cell lysates, and clarified bulk (e.g., clarified cell culture supernatant).

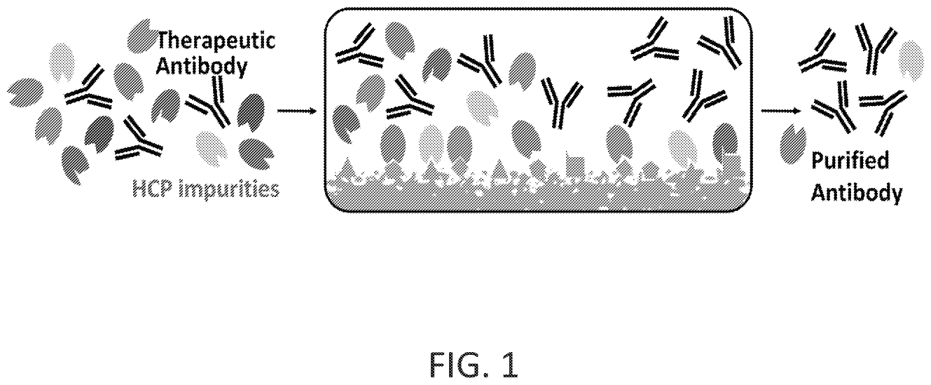

In response to the challenges of efficiently and effectively removing impurities (e.g., host cell proteins) from a cell culture fluid during biomanufacturing processes, embodiments of the present disclosure have established novel and scalable technologies for continuous target molecule purification (e.g., biologic purification). In accordance with these embodiments, the concept of “flow-through affinity chromatography” involves use of an ensemble of synthetic peptide ligands that capture a spectrum of HCPs present in a cell culture harvest without retaining the target product (FIG. 1). This approach was established by creating a chromatographic adsorbent with the peptide ligand ensemble (LigaGuard™) for purifying, for example, polyclonal and monoclonal IgGs from complex sources. This system achieved up to 1.3-log HCP reduction and about 85% mAb yield from recombinant fluids, demonstrating a superior HCP clearance in flow-through mode compared to commercial adsorbents such as Capto Adhere and SuperQ. Importantly, proteomics analysis of the effluents produced demonstrated a removal of persistent HR-HCPs, including HSP90, Clusterin, Vimentin, Cathepsins B/D, Histone H2B, etc. Further evaluation with other CHO cell culture harvests, however, indicated that the adsorbent can be improved further to remove other problematic HCPs. These observations prompted further development of this technology, ultimately resulting in a 2^{nd} generation LigaGuard™ resin with improved HCP binding capacity and selectivity.

To demonstrate the effectiveness of this improved approach, a systematic comparison was performed for the first (G.1) and second (G.2) generation LigaGuard™ resins using a panel of six CHO cell culture harvests featuring different mAb subclasses, titers, HCP composition, and concentrations. First, the HCP binding capacity of both LigaGuard™ resins was evaluated in static and dynamic conditions using a null (no mAb product) CHO-S cell culture fluid. The adsorbents exhibited comparable binding capacity, with maximum binding capacity (Qₘₐₓ) between 28 and 30 mg per mL of resin and breakthrough values (DBC_{10%}) between 16 and 22 mg/mL. A distinct difference was observed during the comparison of mAb recovery and in the logarithmic reduction values (LRVs) of HCPs from the industrial CHO cell culture harvests: while G.1 LigaGuard™ limited mAb yield to 75-89% and HCP LRV to 1.3, G.2 LigaGuard™ afforded up to 96% yield and 2 HCP LRV at short residence times (1 min). Proteomics analysis of the effluents from G.2 LigaGuard™ demonstrated the effective removal of persistent immunogenic HCPs, including cathepsins, histones, glutathione-S transferase, and lipoprotein lipases. Finally, a downstream purification segment was constructed by pairing G.2 LigaGuard™ and an affinity adsorbent, namely, Protein A-based Toyopearl AF-rProtein A-650F resin or LigaTrap® Human IgG resin, in series, which afforded a global mAb yield of 85%, and remarkable HCP and DNA LRVs>4. Collectively, these results demonstrate the feasibility of LigaGuard™ resin in next generation mAb manufacturing processes.

As described further herein, next-generation manufacturing of target biologics (e.g., therapeutic monoclonal antibodies) will likely involve continuous processes featuring single-use/disposable adsorbents, small footprint, and minimal volume of aqueous buffers. These characteristics (i) enable process intensification, (ii) accelerate product delivery to clinics—and potentially shorten “bench-to-clinic” time for newer biotherapeutics, and (iii) reduce the environmental impact of biomanufacturing. In this context, a crucial role will be played by the downstream pipeline—namely, the segment of the bioprocess devoted to the purification of the biological product. Specifically, chromatographic adsorbents operating in continuum—and, ideally, in flow-through mode—are ideally suited to continuous and rapid purification. This paradigm is based on flowing a fluid stream containing the biological product throughout a series of adsorbents wherein the impurities are captured and the product flows through unbound.

However, implementing this purification paradigm presents major challenges. The “impurities” to be captured are highly diverse in terms of titer, physicochemical/biomolecular properties (e.g., hydrodynamic radius, chemical composition, isoelectric point, amphiphilicity, secondary/tertiary structure, etc.), and mechanism of toxicity (e.g., direct trigger of an immunogenicity response, denaturing or degrading the product into toxic or immunogenic byproducts, etc.). Accordingly, designing adsorbents that capture rapidly and effective the entire host of process related impurities including, but not limited to, host cell proteins (HCPs) and DNA, endotoxins, adventitious agents (viral and bacterial contaminants), has not yet been achieved.

Particularly difficult and, hence, heavily researched upon both in academia and in the industry, is the clearance of HCPs. Very small (e.g., media components) and large (e.g., viruses and bacteria, and fragments thereof) contaminants can in fact be removed by relying on size-exclusion/filtration methods; analogously, DNA and RNA can be easily removed by ion exchange chromatography, relying on their strong negative charge and homogeneous physicochemical properties. HCPs, on the other hand, are much more diverse and dangerous to the patient's health. Specific HCPs that are known to pose a particular threat to the patient's health have reportedly caused the recall of batches of approved mAbs or failure/interruption of clinical trials of experimental mAbs.

Embodiments of the present disclosure have established flow-through purification of a target biologic (e.g., mAb purification). In particular, embodiments of the present disclosure include “affinity flow-through chromatography,” which involves the identification and use of an ensemble of synthetic peptide ligands that are capable of capturing the whole spectrum of HCPs present in monoclonal- and polyclonal-containing recombinant cell culture fluids (e.g., Chinese Hamster Cell (CHO), Human Embryonic Kidney (HEK), Pichia pastoris, and the like), without retaining the product.

Previous work (i.e., International journal of molecular sciences 20.7 (2019): 1729; Biotechnology and bioengineering 117.2 (2020): 438-452; Separation and Purification Technology 257 (2021): 117890) focused on the development and characterization of the ensemble of peptide ligands. In particular, the present disclosure analyzed the effluents (i.e., flow-through chromatographic fractions collected at regular time intervals) obtained via continuous injection of clarified CHO cell culture harvest through a LigaGuard™ adsorbent at different values of residence time (i.e., ratio of flow rate of the feedstock to the volume of the adsorbent through which it is injected) to determine (i) the recovery of the mAb product and (ii) the clearance of CHO HCPs. The clearance of HCPs was measured both (ii. 1) using cell HCP-specific ELISA kits, which returned single value of “global” HCP removal (reported as logarithmic removal value and calculated as the log₁₀ of the ratio of the HCP amount in the feedstock and the HCP amount in the flow-through fractions); or (ii.2) by proteomics via mass spectrometry, which returned an array of “individual” HCP removal values. The latter is becoming increasingly popular among industries and regulatory agencies to validate the clearance of HCPs, in particular those HCP species that are difficult to remove via traditional chromatographic technology and pose a specific threat to the patient's health.

This previous work identified and developed this ensemble of peptide ligands, which includes peptides GSRYRY (SEQ ID NO: 6), RYYYAI (SEQ ID NO: 7), AAHIYY (SEQ ID NO: 8), IYRIGR (SEQ ID NO: 9), HSKIYK (SEQ ID NO: 10), ADRYGH (SEQ ID NO: 11), DRIYYY (SEQ ID NO: 12), DKQRII (SEQ ID NO: 13), RYYDYG (SEQ ID NO: 14), YRIDRY (SEQ ID NO: 15), HYAI (SEQ ID NO: 16), FRYY (SEQ ID NO: 17), HRRY (SEQ ID NO: 18), RYFF (SEQ ID NO: 19), DKSI (SEQ ID NO: 20), DRNI (SEQ ID NO: 21), HYFD (SEQ ID NO: 22), and YRFD (SEQ ID NO: 23), and any derivatives or variants thereof. These embodiments are disclosed in PCT Patent Application Publication WO 2020/112906, filed on Nov. 26, 2019, which is herein incorporated by reference in its entirety. Additionally, in some embodiments, nine of these peptides comprise a first generation LigaGuard™ adsorbent, and include GSRYRY (SEQ ID NO: 6), RYYYAI (SEQ ID NO: 7), AAHIYY (SEQ ID NO: 8), IYRIGR (SEQ ID NO: 9), HSKIYK (SEQ ID NO: 10), DKSI (SEQ ID NO: 20), DRNI (SEQ ID NO: 21), HYFD (SEQ ID NO: 22), and YRFD (SEQ ID NO: 23), and any derivatives or variants thereof.

While the ELISA-based values of “global” HCP removal obtained for different mAb-containing CHO harvests provided by different industrial collaborators using the first generation LigaGuard™ adsorbent were satisfactory, the proteomics analysis of the effluents revealed that some challenging HCPs were not effectively removed. Therefore, embodiments of the present disclosure further enhanced the capturing activity of the first generation peptide composition by identifying an additional five peptides that include EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), and any derivatives or variants thereof. As described further herein, this peptide composition is capable of binding a broader range of HCP contaminants from cell culture fluids and enhances the purification capability of the originally discovered peptide composition. As would be recognized by one of ordinary skill in the art based on the present disclosure, these peptides can be used in any combination or grouping to provide enhanced purification of a target biologic.

### 2. Compositions and Methods for Removing Process and Product-Related Impurities

a. Compositions

The growing application of advanced analytics in the biomanufacturing of therapeutic monoclonal antibodies (mAb) has highlighted challenges connected to the clearance of host cell protein (HCP) impurities. Of special concern in biomanufacturing of mAbs is the removal of “persistent” HCPs, namely species that co-elute with the mAb product from the Protein A capture step and can escape the subsequent polishing steps. Persistent HCPs comprise some immunogenic and mAb-degrading proteins, which represent a threat to patient health and a burden to the biopharmaceutical industry. In response to this challenge, an ensemble of peptide ligands was developed that target the whole spectrum of HCPs in Chinese Hamster Ovary (CHO) cell culture fluids and applied them to mAb purification via flow-through affinity chromatography. In this present disclosure, the development of the adsorbent (LigaGuard™) that enables enhanced recovery and purity of mAb products was further improved. First, the binding capacity of CHO HCPs was evaluated in both static and dynamic conditions. LigaGuard™ featured a remarkable equilibrium capacity of about 30 mg per mL of resin, as well as 10% breakthrough capacities up to 16 and 22 mg/mL, respectively at 1- and 2-minute residence times. LigaGuard™ was then evaluated against a panel of industrial CHO cell culture harvests featuring different mAb titers (1-9 mg/mL), properties, total HCP concentrations (0.3-0.6 mg/mL) and their composition. LigaGuard™ afforded a consistently high HCP clearance, with logarithmic removal values (LRVs) up to 2. Proteomics analysis of the effluents confirmed the removal of persistent immunogenic HCPs, including cathepsins, histones, glutathione-S transferase, and lipoprotein lipases. When implemented prior to an affinity capture step, LigaGuard™, especially G.2 as described in this present disclosure, enabled a global mAb yield of 85%, and remarkable HCP and DNA LRVs>4, thus demonstrating its feasibility in next generation mAb manufacturing processes.

In accordance with these embodiments, the present disclosure provides compositions and methods for purifying a target biologic from one or more product- and/or process-related impurities or contaminants. In some embodiments, the compositions and methods disclosed herein facilitate the flow-through purification and isolation of a target biologic (e.g., antibody, vector construct, etc.) from the one or more product- and/or process-related impurities or contaminants. The composition comprises one or more peptide ligands, each of which can bind with a greater affinity to one or more product- and/or process-related impurities or contaminants than to the one or more target biologics. In some embodiments, the one or more peptide ligands bind one or more host cell proteins (HCP), thereby purifying the target biologic.

The one or more target biologic can be any suitable biological target. For example, the target biologic may be a polypeptide, a protein, an oligonucleotide, a polynucleotide, a virus or a viral capsid, a portion of the viral capsid, a cell or a cell organelle, or a small molecule. In some embodiments, the target biologic is a protein, such as an antibody, an antibody fragment, an antibody-drug conjugate, a drug-antibody fragment conjugate, a Fc-fusion protein, a hormone, an anticoagulant, a blood coagulation factor, a growth factor, a morphogenic protein, a therapeutic enzyme, an engineered protein scaffold, an interferon, an interleukin, or a cytokine.

As would be understood by one of ordinary skill in the art based on the present disclosure, the target biologic can be any protein, peptide, or polypeptide produced in a cell, including any endogenous, exogenous, or recombinant proteins produced by a cell, and the methods and compositions described herein can facilitate their purification from HCPs.

In other embodiments, the target biologic can be a virus, viral capsid, or viral vector propagated in a cell. In some embodiments, such viruses, viral capsids, or viral vectors are engineered to deliver genetic material into cells for gene therapy, oncolytic applications, or vaccination; therefore, the various embodiments of the present disclosure can be used to purify the target biologic viruses, viral capsids, or viral vectors before they are administered to a cell or a subject. For example, as described further herein, the target biologic can be a retrovirus (RV), an adenovirus (AV), an adeno-associated virus (AAV), a lentivirus (LV), a baculovirus, or a herpes simplex virus (HSV). As would be understood by one of ordinary skill in the art based on the present disclosure, the target biologic can be any viral vector produced in a cell, and the methods and compositions described herein can facilitate their purification from HCPs.

In some embodiments, the target biologic can be a cell in a stem cell, a progenitor cell, or an immune effector cell. In some embodiments, the immune effector cell includes, but is not limited to, a T cell or a Natural Killer (NK) cell, including immune effector cells engineered to include a chimeric antigen receptor (CAR), such as CAR-T cells and CAR-NK cells. In some embodiments, the target biologic can be an extracellular vesicle or an exosome.

The one or more product- and/or process-related impurities or contaminants can be any protein, peptide, polypeptide, and/or nucleic acid that is not desirable in a purified composition comprising a target biologic. For example, product- and/or process-related impurities can include any fragments or aggregates of the target biologic that are not desired in a purified composition. In other embodiments, the product- and/or process-related impurities can include an intact target biologic that has undergone a chemical or biochemical modification (e.g., enzyme modification of the amino acid sequence of the target biologic or its profile of post-translational modifications), or an intact target biologic that has become associated with an impurity (e.g., and has been rendered inactive).

In accordance with these embodiments, the at least one peptide ligand binds at least one HCP, at least one host cell nucleic acid, aggregates of the target biologic, and/or an impurity derived from the target biologic. The one or more HCPs can be any host cell protein which one would want to remove from a mixture and is independently selected from the proteome of the host cell expressing the one or more target biologics. Examples of host cell proteins include, but are not limited to, acidic ribosomal proteins, biglycan, cathepsins, clusterin, heat shock proteins, nidogen, peptidyl-prolyl cis-trans isomerase, protein disulfide isomerase, SPARC, thrombospondin-1, vimentin, histones, endoplasmic reticulum chaperone BiP, legumain, serine protease HTRA1, and putative phospholipase B-like protein.

In accordance with these embodiments, the composition includes at least one peptide ligand that is at least four amino acids in length and comprises at least one basic amino acid and at least one hydrophilic amino acid. In some embodiments, the at least one peptide ligand comprises a hydrophobic amino acid or a positively charged amino acid at the second amino acid position (e.g., X₁-H₂-X₃-X₄-; where X is an amino acid and His a hydrophobic amino acid; and X₁-P₂-X₃-X₄-; where X is an amino acid and P is a positively charged amino acid). In some embodiments, the at least one peptide ligand comprises a hydrophobic amino acid or a negatively charged amino acid at the fourth amino acid position (e.g., X₁-X₂-X₃-H₄-; where X is an amino acid and His a hydrophobic amino acid; and X₁-X₂-X₃-N₄-; where X is an amino acid and Nis a negatively charged amino acid).

In some embodiments, the at least one peptide ligand comprises an acidic amino acid directly adjacent to a hydrophobic amino acid or a negatively charged amino acid. In some embodiments, the at least one peptide ligand comprises a polar amino acid directly adjacent to cationic amino acid or an aliphatic amino acid. In some embodiments, a negatively charged amino acid in the peptide ligand is: (i) not directly adjacent to a polar amino acid; (ii) directly adjacent to an aliphatic amino acid or an aromatic amino acid; and/or (iii) directly adjacent to a positively charged amino acid. In some embodiments, an aromatic amino acid in the peptide ligand is: (i) directly adjacent to an aliphatic amino acid; (ii) directly adjacent to an anionic amino acid; and/or (iii) directly adjacent to a cationic amino acid.

In accordance with these embodiments, hydrophobicity of an amino acid can be determined by any means known in the art, such as with a hydrophilicity plot. A hydrophobicity plot is a quantitative analysis of the degree of hydrophobicity or hydrophilicity of amino acids of a protein. It can be used to characterize or identify possible structure or domains of a protein. Generally, the plot has amino acid sequence of a protein on its x-axis, and degree of hydrophobicity and hydrophilicity on its y-axis. There are a number of methods to measure the degree of interaction of polar solvents such as water with specific amino acids. For example, the Kyte-Doolittle scale indicates hydrophobic amino acids, whereas the Hopp-Woods scale measures hydrophilic residues. Analyzing the shape of the plot provides information about partial structure of the protein. The Hopp-Woods hydrophilicity scale of amino acids can be used to rank the amino acids in a protein according to their water solubility in order to search for surface locations on proteins, and especially those locations that tend to form strong interactions with other macromolecules such as proteins, DNA, and RNA.

In some embodiments, amino acids that are considered to be hydrophobic (have hydrophobic side chains) include glycine (Gly), alanine (Ala, A), valine (Val, V), leucine (Leu, L), isoleucine (Ile, I), proline (Pro, P), phenylalanine (Phe, F), methionine (Met, M), and tryptophan (Trp, W). Additionally, amino acids that are considered to be positively charged (cationic) include lysine (Lys, K), arginine (Arg, R) and histidine (His, H) (basic side chains), and amino acids that are considered to be negatively charged (anionic) include aspartic acid (Asp, D) and glutamic acid (Glu, E) (acidic side chains). Amino acids considered to be polar amino acids include serine (Ser, S), threonine (Thr, T), cysteine (Cys, C), asparagine (Asn, N), glutamine (Gln, Q), and tyrosine (Tyr, Y). Amino acids considered to be aliphatic amino acids include isoleucine (Ile, I), leucine (Leu, L), proline (Pro, P), and valine (Val, V). And amino acids considered to be aromatic amino acids include tryptophan (Trp, W), tyrosine (Tyr, Y), and phenylalanine (Phe, F).

As would be recognized by one of ordinary skill in the art based on the present disclosure, the peptide ligands provided herein can be conjugated to a linker. In some embodiments, the linker can facilitate display of a peptide ligand onto a solid support, which allows for better capture of an HCP, for example. In other embodiments, the peptide ligands provided herein are not conjugated to a linker, but can still be bind to HCPs and be removed from a cell culture fluid through other means. In some embodiments, the one or more peptide ligands comprise a linker on the C-terminus of the peptide. The C-terminus linker comprise a linker according to the following structure: Glyₙ or a [Gly-Ser-Gly]ₘ, wherein 6≥n≥1 and 3≥m≥1. The C-terminus linker can be any suitable linker including, but not limited to GSG and GGG.

In some embodiments, the at least one peptide ligand exhibits a K_{D} from about 10⁻¹⁰ M to about 10⁻³ M for the HCP, the host cell nucleic acid, and/or the target biologic aggregate. In some embodiments, the at least one peptide ligand exhibits a K_{D} from about 10⁻⁹ M to about 10⁻³ M for the HCP, the host cell nucleic acid, and/or the target biologic aggregate. In some embodiments, the at least one peptide ligand exhibits a K_{D} from about 10⁻⁸ M to about 10⁻³ M for the HCP, the host cell nucleic acid, and/or the target biologic aggregate. In some embodiments, the at least one peptide ligand exhibits a K_{D} from about 10⁻⁷ M to about 10⁻³ M for the HCP, the host cell nucleic acid, and/or the target biologic aggregate. In some embodiments, the at least one peptide ligand exhibits a K_{D} from about 10⁻⁶ M to about 10⁻³ M for the HCP, the host cell nucleic acid, and/or the target biologic aggregate. In some embodiments, the at least one peptide ligand exhibits a K_{D} from about 10⁻⁵ M to about 10⁻³ M for the HCP, the host cell nucleic acid, and/or the target biologic aggregate. In some embodiments, the at least one peptide ligand exhibits a K_{D} from about 10⁻⁴ M to about 10⁻³ M for the HCP, the host cell nucleic acid, and/or the target biologic aggregate. In some embodiments, the at least one peptide ligand exhibits a K_{D} that is lower than about 10⁻⁵ M for the HCP, the host cell nucleic acid, and/or the target biologic aggregate. In some embodiments, the at least one peptide ligand exhibits a K_{D} from about 10⁻¹⁰ M to about 10⁻⁴ M for the HCP, the host cell nucleic acid, and/or the target biologic aggregate. In some embodiments, the at least one peptide ligand exhibits a K_{D} from about 10⁻⁹ M to about 10⁻³ M for the HCP, the host cell nucleic acid, and/or the target biologic aggregate. In some embodiments, the at least one peptide ligand exhibits a K_{D} from about 10⁻⁹ M to about 10⁻⁴ M for the HCP, the host cell nucleic acid, and/or the target biologic aggregate. In some embodiments, the at least one peptide ligand exhibits a K_{D} from about 10⁻⁹ M to about 10⁻⁵ M for the HCP, the host cell nucleic acid, and/or the target biologic aggregate. In some embodiments, the at least one peptide ligand exhibits a K_{D} from about 10⁻⁸ M to about 10⁻³ M for the HCP, the host cell nucleic acid, and/or the target biologic aggregate. In some embodiments, the at least one peptide ligand exhibits a K_{D} from about 10⁻⁸ M to about 10⁻⁴ M for the HCP, the host cell nucleic acid, and/or the target biologic aggregate. In some embodiments, the at least one peptide ligand exhibits a K_{D} from about 10⁻⁸ M to about 10⁻⁵ M for the HCP, the host cell nucleic acid, and/or the target biologic aggregate. In some embodiments, the at least one peptide ligand exhibits a K_{D} from about 10⁻⁷ M to about 10⁻³ M for the HCP, the host cell nucleic acid, and/or the target biologic aggregate. In some embodiments, the at least one peptide ligand exhibits a K_{D} from about 10⁻⁷ M to about 10⁻⁴ M for the HCP, the host cell nucleic acid, and/or the target biologic aggregate. In some embodiments, the at least one peptide ligand exhibits a K_{D} from about 10⁻⁷ M to about 10⁻⁵ M for the HCP, the host cell nucleic acid, and/or the target biologic aggregate. In some embodiments, the at least one peptide ligand exhibits a K_{D} from about 10⁻⁶ M to about 10⁻³ M for the HCP, the host cell nucleic acid, and/or the target biologic aggregate. In some embodiments, the at least one peptide ligand exhibits a K_{D} from about 10⁻⁶ M to about 10⁻⁴ M for the HCP, the host cell nucleic acid, and/or the target biologic aggregate. In some embodiments, the at least one peptide ligand exhibits a K_{D} from about 10⁻⁶ M to about 10⁻⁵ M for the HCP, the host cell nucleic acid, and/or the target biologic aggregate.

In some embodiments, the at least one peptide ligand is no more than 15 amino acids in length. In some embodiments, the at least one peptide ligand is no more than 14 amino acids in length. In some embodiments, the at least one peptide ligand is no more than 13 amino acids in length. In some embodiments, the at least one peptide ligand is no more than 12 amino acids in length. In some embodiments, the at least one peptide ligand is no more than 11 amino acids in length. In some embodiments, the at least one peptide ligand is no more than 10 amino acids in length. In some embodiments, the at least one peptide ligand is from about 4 to about 15 amino acids in length. In some embodiments, the at least one peptide ligand is from about 5 to about 15 amino acids in length. In some embodiments, the at least one peptide ligand is from about 6 to about 15 amino acids in length. In some embodiments, the at least one peptide ligand is from about 7 to about 15 amino acids in length. In some embodiments, the at least one peptide ligand is from about 8 to about 15 amino acids in length. In some embodiments, the at least one peptide ligand is from about 9 to about 15 amino acids in length. In some embodiments, the at least one peptide ligand is from about 10 to about 15 amino acids in length. In some embodiments, the at least one peptide ligand is from about 4 to about 14 amino acids in length. In some embodiments, the at least one peptide ligand is from about 4 to about 13 amino acids in length. In some embodiments, the at least one peptide ligand is from about 4 to about 12 amino acids in length. In some embodiments, the at least one peptide ligand is from about 4 to about 11 amino acids in length. In some embodiments, the at least one peptide ligand is from about 4 to about 10 amino acids in length. In some embodiments, the at least one peptide ligand is from about 5 to about 15 amino acids in length. In some embodiments, the at least one peptide ligand is from about 5 to about 10 amino acids in length. In some embodiments, the at least one peptide ligand is from about 5 to about 15 amino acids in length. In some embodiments, the at least one peptide ligand is from about 10 to about 15 amino acids in length. In some embodiments, the at least one peptide ligand is from about 7 to about 14 amino acids in length.

In some embodiments, the cell culture fluid comprises a supernatant and/or a cellular lysate. In some embodiments, the cell culture fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof. In some embodiments, the cell culture fluid is derived from HEK293 cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof. In some embodiments, the cell culture fluid is derived from yeast cells. In some embodiments, the yeast cells are selected from the group consisting of P. pastoris, S. cerevisiae, and S. boulardii, or any derivatives or variants thereof. In some embodiments, the cell culture fluid is derived from a virus production cell line. In some embodiments, the virus production cell line is selected from the group consisting of MDCK-S, MDCK-A, Vero cells, LLC-MK2D, PER.C6, EB66, and AGE1.CR cells, or any derivatives or variants thereof.

In some embodiments, the cell culture fluid comprises a pH from about 3.0 to about 9.0. In some embodiments, the cell culture fluid comprises a pH from about 4.0 to about 9.0. In some embodiments, the cell culture fluid comprises a pH from about 5.0 to about 9.0. In some embodiments, the cell culture fluid comprises a pH from about 6.0 to about 9.0. In some embodiments, the cell culture fluid comprises a pH from about 7.0 to about 9.0. In some embodiments, the cell culture fluid comprises a pH from about 3.0 to about 8.0. In some embodiments, the cell culture fluid comprises a pH from about 3.0 to about 7.0. In some embodiments, the cell culture fluid comprises a pH from about 3.0 to about 6.0. In some embodiments, the cell culture fluid comprises a pH from about 4.0 to about 8.0. In some embodiments, the cell culture fluid comprises a pH from about 5.0 to about 7.0.

In some embodiments, the cell culture fluid comprises a conductivity of about 1 to about 50 mS/cm. In some embodiments, the cell culture fluid comprises a conductivity of about 5 to about 50 mS/cm. In some embodiments, the cell culture fluid comprises a conductivity of about 10 to about 50 mS/cm. In some embodiments, the cell culture fluid comprises a conductivity of about 15 to about 50 mS/cm. In some embodiments, the cell culture fluid comprises a conductivity of about 20 to about 50 mS/cm. In some embodiments, the cell culture fluid comprises a conductivity of about 30 to about 50 mS/cm. In some embodiments, the cell culture fluid comprises a conductivity of about 40 to about 50 mS/cm. In some embodiments, the cell culture fluid comprises a conductivity of about 1 to about 40 mS/cm. In some embodiments, the cell culture fluid comprises a conductivity of about 1 to about 30 mS/cm. In some embodiments, the cell culture fluid comprises a conductivity of about 1 to about 20 mS/cm. In some embodiments, the cell culture fluid comprises a conductivity of about 1 to about 15 mS/cm. In some embodiments, the cell culture fluid comprises a conductivity of about 10 to about 40 mS/cm. In some embodiments, the cell culture fluid comprises a conductivity of about 20 to about 30 mS/cm.

In accordance with the above embodiments, the at least one peptide ligand can comprise at least 2 peptide ligands, at least 3 peptide ligands, at least 4 peptide ligands, at least 5 peptide ligands, at least 6 peptide ligands, at least 7 peptide ligands, at least 8 peptide ligands, at least 9 peptide ligands, at least 10 peptide ligands, at least 11 peptide ligands, at least 12 peptide ligands, at least 13 peptide ligands, at least 14 peptide ligands, at least 15 peptide ligands, at least 16 peptide ligands, at least 17 peptide ligands, at least 18 peptide ligands, at least 19 peptide ligands, at least 20 peptide ligands, at least 21 peptide ligands, at least 22 peptide ligands, at least 23 peptide ligands, at least 24 peptide ligands, at least 25 peptide ligands, at least 26 peptide ligands, at least 27 peptide ligands, at least 8 peptide ligands, at least 29 peptide ligands, or at least 30 peptide ligands. In some embodiments, the one or more peptide ligands comprise different amino acid sequences.

In some embodiments, the at least one peptide ligand is selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof. In some embodiments, the at least one peptide ligand comprises at least two peptide ligands selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof. In some embodiments, the at least one peptide ligand comprises at least three peptide ligands selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof. In some embodiments, the at least one peptide ligand comprises at least four peptide ligands selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof. In some embodiments, the composition comprises EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof.

In some embodiments, the composition further comprises at least one peptide ligand selected from the group consisting of: GSRYRY (SEQ ID NO: 6), RYYYAI (SEQ ID NO: 7), AAHIYY (SEQ ID NO: 8), IYRIGR (SEQ ID NO: 9), HSKIYK (SEQ ID NO: 10), ADRYGH (SEQ ID NO: 11), DRIYYY (SEQ ID NO: 12), DKQRII (SEQ ID NO: 13), RYYDYG (SEQ ID NO: 14), YRIDRY (SEQ ID NO: 15), HYAI (SEQ ID NO: 16), FRYY (SEQ ID NO: 17), HRRY (SEQ ID NO: 18), RYFF (SEQ ID NO: 19), DKSI (SEQ ID NO: 20), DRNI (SEQ ID NO: 21), HYFD (SEQ ID NO: 22), and YRFD (SEQ ID NO: 23), or any derivatives or variants thereof. In some embodiments, the composition further comprises at least one peptide ligand selected from the group consisting of: GSRYRY (SEQ ID NO: 6), RYYYAI (SEQ ID NO: 7), AAHIYY (SEQ ID NO: 8), IYRIGR (SEQ ID NO: 9), HSKIYK (SEQ ID NO: 10), DKSI (SEQ ID NO: 20), DRNI (SEQ ID NO: 21), HYFD (SEQ ID NO: 22), and YRFD (SEQ ID NO: 23), and any derivatives or variants thereof.

In accordance with these embodiments, the compositions of the present disclosure can be used in the production of any biologic, including but not limited to, biologic molecules such as antibodies and antibody fragments (e.g., single-chain variable fragments (scFv), single-chain antibodies (scAb), and fragment antigen binding molecules (Fab fragments), diabodies, glycoengineered antibodies, bi-specific antibodies, antibody-drug conjugates, as well as any combinations, derivatives, variants, and fusions thereof. For example, the peptide compositions of the present disclosure can be used to purify any of the currently available therapeutic antibodies, including but not limited to, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), Inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ustekinumab (Stelara), infliximab, and bevacizumab. In some embodiments, the present disclosure includes a composition comprising any of the peptide ligands disclosed herein, and one of the above-recited therapeutic antibodies.

b. Adsorbents

Further described herein are adsorbents comprising a composition as described above, where each peptide of the composition is conjugated to a support. Supports may comprise, but are not limited to, particles, beads, plastic surfaces, resins, fibers, and/or membranes. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate, polyolefin, polyester, polysaccharide, iron oxide, silica, titania, and/or zirconia. In some embodiments, supports may include microparticles and/or nanoparticles. Each support may be made out of any suitable material including, but not limited to, synthetic or natural polymers, metals, and metal oxides. Some supports may be magnetic, such as a magnetic bead, microparticle and/or nanoparticle. Suitable synthetic polymers include, but are not limited to, polymethacrylate, polyethersulfone, and polyethyleneglycol. Suitable natural polymers include, but are not limited to, cellulose, agarose, and chitosan. Suitable metal oxides include, but are not limited to, iron oxide, silica, titania, and zirconia. Further described herein are adsorbents comprising a composition as described above conjugated to a support.

In some embodiments, the adsorbent comprises a single type of support made from a single type of support material, where all of the peptides in the composition are conjugated to supports formed of the single type of support material. In these embodiments, the composition may comprise one or more different types of peptides, each conjugated to the single type of support made from the single type of support material. In other embodiments, the adsorbent comprises a plurality of types of support. Each type of support may be made of the same type of support material or different types of support materials. In these embodiments, the composition may comprise one or more different types of peptides, each conjugated to a different type of support. In still other embodiments, the peptides of the composition can be conjugated to a soluble compound, for example stimuli-responsive polymer chains to remove HCPs by affinity precipitation.

c. Methods

As described further herein, the present disclosure also provides improved methods for purifying a target biologic from a biological fluid comprising and one or more product- and/or process-related impurities or contaminants, as compared to currently used methods. In some embodiments, the method includes contacting a composition comprising any of the peptide ligands described herein with a cell culture fluid, and collecting the cell culture fluid in flow-through mode, with the cell culture fluid comprising the target biologic. In some embodiments, the at least one peptide ligand binds a host cell protein (HCP), a host cell nucleic acid, and/or an aggregate of the target biologic in a retentate.

Further described herein are methods for removing one or more host cell proteins from a mixture comprising the one or more host cell proteins and one or more target biologics. The methods comprise contacting the mixture with a composition or adsorbent described herein. In one embodiment, the contacting between the composition or adsorbent and the mixture results in the binding of the one or more host cell proteins to the composition or adsorbent. In this embodiment, the one or more host cell proteins has a higher binding affinity for the composition, as compared to the one or more target biologics. This results in the preferred binding of the composition to the one or more host cell proteins as compared to the one or more target molecules.

The methods of the present disclosure can further comprise washing the composition or adsorbent to remove one or more unbound target biologics into a supernatant or mobile phase; and then collecting the supernatant or mobile phase containing the one or more unbound target biologics. In an embodiment, the washing step can also occur after the contacting step and after the collection of the supernatant or mobile phase.

In some embodiments, the method can be performed under any binding conditions suitable for use with the composition or adsorbent, including both static binding conditions and dynamic binding conditions. In some embodiments the unbound target biologics are collected into a supernatant when the methods are performed under static binding conditions. In some embodiments the unbound target biologics are collected into a mobile phase when the methods are performed under dynamic binding conditions. The methods of the present disclosure can optionally include flow-through chromatography and weak partition chromatography.

The binding affinity of the compositions and/or adsorbent for the host cell proteins, as compared to the one or more target molecules, can be altered by changes in the following: properties and concentration of the one or more target proteins; the properties and concentration of the host cell proteins; the composition, concentration, and pH of the mixture; and/or the loading conditions and residence time of the contacting and washing steps. Any of these variables can be changed to variables which are suitable according to the methods of the present disclosure and result in increased or decreased binding affinity as required for the present disclosure.

In some embodiments, the contacting step comprises a high ionic strength binding buffer or low ionic strength binding buffer. A low ionic strength binding buffer comprises a buffer of between 1-50 mM NaCl. In one embodiment the low ionic strength binding buffer comprises 20 mM NaCl. A high ionic strength binding buffer comprises a buffer of between 100-500 mM NaCl. In one embodiment the low ionic strength binding buffer comprises 150 mM NaCl.

In some embodiments, the contacting step can comprise a low pH buffer of between pH 5-9. In some embodiments, the contacting step can comprise a low pH buffer of between pH 5-8. In some embodiments, the contacting step can comprise a low pH buffer of between pH 5-7. In some embodiments, the contacting step can comprise a low pH buffer of between pH 6-9. In some embodiments, the contacting step can comprise a low pH buffer of between pH 6-8. In some embodiments, the contacting step can comprise a low pH buffer of between pH 6-7. In some embodiments, the contacting step can comprise a low pH buffer of between pH 7-9. In some embodiments, the contacting step can comprise a low pH buffer of between pH 7-8.

As would be appreciated by one of ordinary skill in the art based on the present disclosure, the methods described herein can be used before or after any purification method typically used to purify and/or isolate a given target molecule. For example, the methods disclosed herein can be used before or after ion exchange chromatography (e.g., cation exchange chromatography, anion exchange chromatography, and/or mixed mode chromatography), before or after affinity chromatography (e.g., Protein A affinity chromatography), and/or before or after size exclusion chromatography or other filtration treatment. In some embodiments, the methods disclosed herein are used after a cell culture fluid has been clarified, but prior to performing a chromatography step (e.g., Protein A affinity chromatography).

In some embodiments, the methods of the present disclosure are particularly suitable for use in the manufacturing of therapeutic antibodies, which can greatly benefit from the adoption of the compositions of peptides and adsorbents of the present disclosure owing to their potential of transforming the downstream process from a pipeline of “batch” chromatographic steps operated in “bind-and-elute” mode to a pipeline of continuous and connected chromatographic train operated in flow-through” mode. However, the methods described herein are also applicable to the purification of other target biologics, such as gene therapy products. These include, for example, viruses for in vivo (e.g., adenovirus and adeno-associated virus) and in vitro (e.g., lentivirus and baculovirus) gene therapy. Unlike proteins, viruses are much larger in size (>20 nm), yet much lower in titer (10¹¹-10¹³ vg/mL, corresponding to μg/mL levels, much lower than the typical mg/mL titer of proteins in cell culture harvests) and often in biochemical stability (e.g., all viruses quickly lose infectivity when exposed to the typical elution conditions (low pH) currently utilized for their purification; specific adeno-associated virus serotypes are very prone to irreversible adhesion and aggregation; lentiviruses are very sensitive to pH variations outside of the physiological range). As a result, affinity-based purification in capture-and-elute mode fails to provide the product yield and quality requested by clinics and biotech companies worldwide. The compositions and methods of the present disclosure circumvent these issues by enabling flow-through purification of viruses. The key benefits of this approach include, but are not limited to, (i) flowing the cell culture fluid from the bioreactor to capture HCPs while excluding the viruses by size (upon adjusting the pore diameter, the HCPs can enter the pores, while the viruses are excluded), thereby improving product recovery; (ii) rapid clearance of HCPs at minimal residence time (upon adjusting the particle diameter), thereby improving product stability; (iii) operating in flow-through mode avoids virus adsorption on the resin and exposure to variations in conductivity and pH (associated to washing/elution buffers in current bind-and-elute affinity purification), thereby reducing product aggregation and preserving its transduction activity.

The production of non-therapeutic proteins forms a large segment of the present economy. Improvement of livestock and crops via genetic engineering (e.g., CRISPR) requires the availability of purified gene-editing enzymes (e.g., Cas9 nucleases). Very often, these proteins feature remarkable biochemical lability, which makes large-scale purification challenging and limits product throughput and quality, thereby increasing drastically the price of these products. Accelerating and simplifying the purification process of these products is an essential contribution towards enabling their widespread use in the future. The compositions and methods of the present disclosure can support the production of non-therapeutic proteins for the biotech/ag-bio industry.

Additional applications of the compositions and methods of the present disclosure include the detection of low-abundance proteins in biological fluids, such as cell culture harvests, plant/tissue extracts, bodily fluids (e.g., blood, serum, plasma, sweat, urine, saliva). In this context, the prevalence of mass spectrometry (MS)-based analytical techniques for process monitoring and diagnostic applications has posed an accent on the need to enrich and/or isolate low-abundance proteins that are often key markers of product quality of disease. MS-based analysis relies on the ionization of the analyte species in the sample: abundant analytes, due to their higher titer, capture most of the electrons, at the expense of low-titer analytes, which become undetected. The compositions and methods of the present disclosure can overcome these limitations by concentrating the HCPs and releasing them in a controlled fashion: (i) all HCPs are initially captured on the adsorbent; (ii) the HCPs are “eluted” using a linear or a step-wise gradient, which progressively releases cohorts of HCPs from the adsorbent and directly into the analytical equipment. The low-abundance proteins are present in the eluted stream at a much higher concentration and are more likely to be detected.

In accordance with these embodiments, the compositions and methods of the present disclosure can be used in the production of any biologic, including but not limited to, biologic molecules such as antibodies (monoclonal and polyclonal) and antibody fragments (e.g., single-chain variable fragments (scFv), single-chain antibodies (scAb), fragment antigen binding molecules (Fab fragments), diabodies, glycoengineered antibodies, bi-specific antibodies, antibody-drug conjugates, as well as any combinations, derivatives, variants, and fusions thereof. For example, the peptide compositions and methods of the present disclosure can be used to purify any of the currently available therapeutic antibodies, including but not limited to, abciximab (Reopro), adalimumab (Humira, Amjevita), alefacept (Amevive), alemtuzumab (Campath), basiliximab (Simulect), belimumab (Benlysta), bezlotoxumab (Zinplava), canakinumab (Ilaris), certolizumab pegol (Cimzia), cetuximab (Erbitux), daclizumab (Zenapax, Zinbryta), denosumab (Prolia, Xgeva), efalizumab (Raptiva), golimumab (Simponi, Simponi Aria), inflectra (Remicade), ipilimumab (Yervoy), ixekizumab (Taltz), natalizumab (Tysabri), nivolumab (Opdivo), olaratumab (Lartruvo), omalizumab (Xolair), palivizumab (Synagis), panitumumab (Vectibix), pembrolizumab (Keytruda), rituximab (Rituxan), tocilizumab (Actemra), trastuzumab (Herceptin), secukinumab (Cosentyx), ustekinumab (Stelara), infliximab, and bevacizumab. In some embodiments, the present disclosure includes a method for purifying any of the above-recited therapeutic antibodies by combining a cell culture fluid comprising one of the above-recited antibodies with a composition comprising any of the peptide ligands disclosed herein.

### 3. EXAMPLES

The accompanying Examples are offered as illustrative as a partial scope and particular embodiments of the disclosure and are not meant to be limiting of the scope of the disclosure.

The future therapeutic mAb manufacturing will most likely rely on next-generation chromatographic adsorbents that are also conducive for continuous operation, with an improved capability of removing HCPs, especially high risk species that have been identified as persistent in current bioprocesses. In this context, embodiments of the present disclosure include a downstream toolbox comprising affordable peptide-based chromatographic adsorbents that purify therapeutic proteins in bind-and-elute mode or via “flow-through affinity chromatography.” The latter (i.e., LigaGuard™) operates by capturing HCPs selectively, while allowing the mAb product to flow through unbound as a clarified cell culture harvest is fed in continuum without a need for prior conditioning.

Clearing HCPs from a cell culture harvest requires chromatographic substrates functionalized with ligands capable of capturing a spectrum of proteins that feature a vast diversity in terms of concentration, physicochemical properties (i.e., hydrodynamic radius, chemical composition, isoelectric point, amphiphilicity, and structure, etc.), and safety profile (e.g., toxicity, immunogenicity, degradation activity to Protein A, the mAb product, and the excipients utilized in the drug formulation, etc.). To accomplish this, an initial ensemble of 9 peptide ligands was developed, referred to as the first generation (G.1) LigaGuard™, by selecting linear 4-mer and 6-mer peptides against a model CHO feedstock comprising human polyclonal IgG and HCPs via dual-fluorescence solid phase library screening. This adsorbent was validated by purifying a therapeutic mAb from a clarified CHO cell culture harvest in flow-through mode, affording good mAb recovery (˜85%) and purity (90%). While demonstrating the feasibility of this purification paradigm, G.1 LigaGuard™ resin was unable to significantly remove a subset of high-risk (HR) HCPs, namely Cathepsin Z, Glutathione-S Transferases, Peroredoxins etc. Therefore, a G.2 LigaGuard™ was developed with superior HCP-capture capacity and selectivity. To this end, five additional peptides designed in silico to target model HCPs via multi-point interactions were introduced.

The results of the present disclosure include a comparative study of G.1 and G.2 LigaGuard™ resins by evaluating process-relevant parameters including their (i) static and dynamic binding capacity for CHO HCPs, (ii) HCP vs. mAb binding selectivity, (iii) mAb recovery and clearance of HCPs from a panel of six industrial CHO cell culture harvests, and (iv) proteomic analysis of the effluents to document effective removal of persistent HR HCPs. The feasibility of G.2 LigaGuard™ resin as an HCP-scrubbing adsorbent for mAb processing was evaluated by quantifying the synergism of HCP removal in combination with Protein A resin.

### Example 1

HCP binding capacity and selectivity of LigaGuard™ resins. Titer and biomolecular diversity of HCPs vary with the cell lines used, cell culture media formulation, operating conditions, longevity of the cell line and time. It is therefore critical to quantify the binding capacity and selectivity of G.1 and G.2 LigaGuard™ resins and identify appropriate loading conditions, namely, the volumetric ratio of cell culture harvest vs. adsorbent volume needed to achieve satisfactory mAb recovery and purity. Accordingly, static and dynamic binding studies were performed in both non-competitive, namely, mAb-free CHO-S cell culture harvest and pure NIST mAb solutions at different titers, and competitive conditions, namely, a null CHO-S solution spiked with NIST mAb.

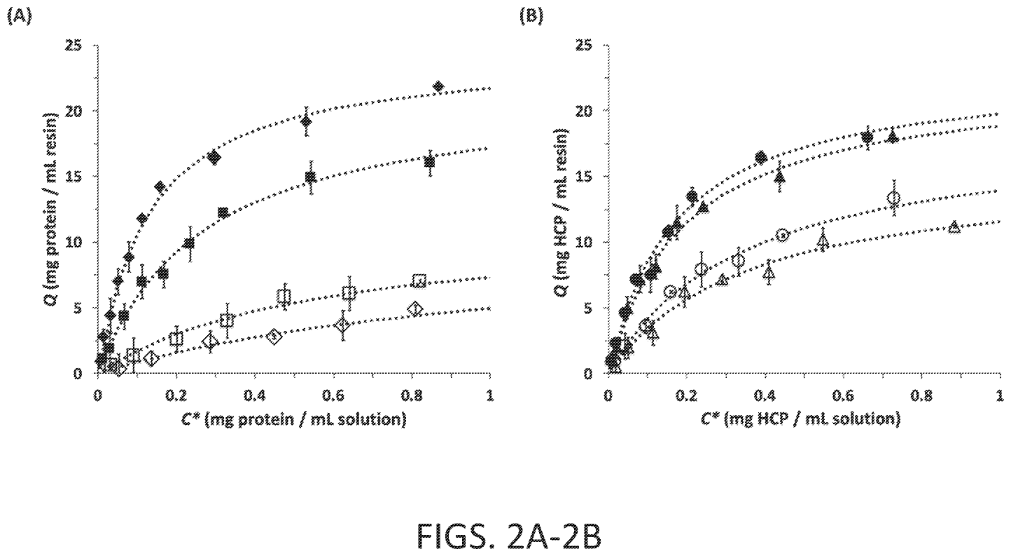

The values of static binding capacity, obtained by fitting the isotherms in FIG. 2 against a Langmuir isotherm (reported in Table 1) offer good insight into the binding strength and selectivity of the ensemble of peptide ligands. The solid markers in FIG. 2A depict the binding affinities of G.1 (squares) and G.2 (diamonds) LigaGuard™ respectively, while their outlined counterparts trend the ligands' performance with a solution of pure mAb. The trends in FIG. 2B describe the performance of both ligand ensembles subjected to a model fluid with varying HCP concentration and mAb concentration solutions-1 mg/mL (circles) and 5 mg/mL (triangles) respectively.

In the absence of the IgG product, G.1 and G.2 LigaGuard™ resins exhibited comparable values of HCP binding capacity, with asymptotic values of capacity (Q_{max,HCPs}) of 21.8 and 24.5 mg of HCPs per mL of resin (FIG. 2A). Furthermore, the micromolar values of K_{D,HCPs} were calculated to be 6.75 μM for G.1 ligands and 3.25 μM for G.2, (note: these values were calculated assuming an average HCP molecular weight of 40 kDa) placing the LigaGuard™ peptides well within the class of affinity ligands. While the values of equilibrium capacity are lower than that characteristic of commercial affinity resin like Protein A, which feature equilibrium capacity ranging between 35-54 mg of IgG per mL of resin [35,36], it should be considered that the titer of HCPs in industrial CHO cell culture harvests is typically comprised between 0.3 and 0.8 g/L, i.e. approximately 1/25^{th}-⅕^{th} of the mAb titer; therefore, with Qₘₐₓ, HCPs˜20-25 mg/mL, LigaGuard™ resins possess sufficient binding capacity to be operated in tandem with Protein A-based resin in a mAb purification process.

Analogously, while their HCP binding strength (K_{D,HCPs}) is milder than that of protein-targeting affinity ligands, it should be considered that the performance of HCP binding ligands depends on both—the titer of individual HCP present in the HCCF and the molar concentration of target epitopes that provides adequate driving force for ligand binding, i.e., presence of at least 1 nM-20 nM. Therefore, while a rigorous value cannot be provided, the inherent HCP-binding strength of LigaGuard™ ligands is substantially higher than the level portrayed by the K_{D,HCPs} derived from the binding isotherms. Conversely, binding capacities and strength of the “competing” IgG species using LigaGuard™ were lower than that observed for HCPs, exhibiting an inherent selectivity towards HCPs. IgG binding may be caused by the lack of target HCPs in the feedstock, which leave all ligands available for non-selective capture of IgG mostly due to electrostatic and hydrophobic interactions. However, it must be considered that with the increase in HCP concentration, these ligands are able to preferentially bind to the impurities, leading to high mAb product recovery and yields as well. As anticipated, when evaluated against pure species, G.2 has a higher selectivity for HCPs than IgG, corroborating the rationale underlying the design of the additional five peptide ligands. In this context, it is also worth noticing that the HCP binding kinetics (k_{on,HCP}˜3.9±0.4 10⁴ M⁻¹s⁻¹; (calculated from the binding kinetics in FIG. 8A assuming an average HCP molecular weight of 40 kDa) was found to be substantially faster than that of IgG (k_{on,mAb}˜8.71±0.1 10³ M⁻¹s⁻¹).

Further information was provided by the static binding studies performed in competitive conditions, which utilized solutions of CHO HCPs spiked with NIST mAb at a constant concentration of either 1 or 5 mg/mL (FIG. 2B). Under these conditions, the HCPs were found to outcompete the mAb in binding to the peptide ligands, initially through a kinetics-governed competition (k_{on,HCP}>K_{on,mAb}) and ultimately through a thermodynamics-governed competition (K_{D,HCPs}<K_{D,mAb}). Most notably, while the Qₘₐₓ, HCPs of G.2 resin decreased by a mere ˜25%, from ˜24 to ˜18 mg per mL, upon introducing mAb at 1 mg/mL in the CHO HCP solution, and down to ˜16 mg per mL upon increasing the mAb titer to 5 mg/mL. Conversely, the Qₘₐₓ, HCPs of G.1 resin dropped from ˜21 mg/mL to 13 mg/mL and ultimately to ˜10 mg per mL upon introducing mAb at 1 and 5 mg/mL, respectively. This demonstrates that G.2 LigaGuard™ resin feature a superior HCP binding capacity and selectivity compared to their G.1 precursor.

Prior work on G.1 LigaGuard™ resin indicated that HCP capture in flow-through mode was affected rather substantially by residence time. Accordingly, the dynamic binding capacity of G.2 LigaGuard™ resin at residence times 0.5, 1, 2, and 5 min was evaluated using an industrial CHO cell culture harvest (mAb titer of 1.38 mg/mL and HCP titer of 0.46 mg/mL). The comparison between values of DBC_{10%} obtained from the breakthrough curves (FIG. 8B) demonstrate that the HCP capture by G.2 LigaGuard™ resin is minimally affected by the flow conditions, decreasing from 17.6 mg per mL at 5 min, to 16.7 and 16.2 mg/mL at 1 and 2 min, and ultimately to 14.3 mg/mL at 0.5 min; this is coherent with the prior observation of rapid binding (high k_{on,HCP}) and high selectivity of this second-generation adsorbent. With the goal of maximizing mAb productivity while achieving best HCP log-reduction, experiments focused on residence times of 1 and 2 min for the remainder of this study.

TABLE 1
Values of static binding capacity (Qₘₐₓ) and affinity (K_{D}) of G.1 and G.2
LigaGuard ™ resin in either non-competitive or competitive conditions.

| | | | | Competitive | | | |
|---|---|---|---|---|---|---|---|
| | Non-competitive | | | 1 mg mAb/mL | | 5 mg mAb/mL | |
| | | K_{D} | Qₘₐₓ | K_{D} HCP | Qₘₐₓ | K_{D} HCP | Qₘₐₓ |
| | Species | (μM) | (mg/mL) | (μM) | (mg/mL) | (μM) | (mg/mL) |
| G.1 | mAb | 14.25 | 11.5 | 8.75 | 18.8 | 8.75 | 15.6 |
| LigaGuard ™ | HCPs | 6.50 | 21.8 | | | | |
| G.2 | mAb | 28.27 | 10.5 | 4.25 | 23.1 | 4.75 | 22.4 |
| LigaGuard ™ | HCPs | 3.25 | 24.5 | | | | |

### Example 2

mAb purification via flow-through affinity chromatography using G.1 and G.2 LigaGuard™ resins. ICH guidelines Q8, Q9 and Q10 provide a well-defined approach to develop processes with high productivity while meeting targets for critical quality attributes, such as biomolecular profile of the mAb product and residual HCP and host DNA titer. Tracking process- or product-related impurities using different—and orthogonal—analytical techniques is now commonplace in the biomanufacturing of therapeutic mAbs and other proteins. Accordingly, in the present disclosure, analytical chromatographic techniques were used (e.g., Protein G) for mAb titer, and size exclusion chromatography was used for product purity assessment. In addition to these, ELISA and proteomics analyses using mass spectrometry (LC-MS/MS) were to evaluate global and individual HCP clearance.

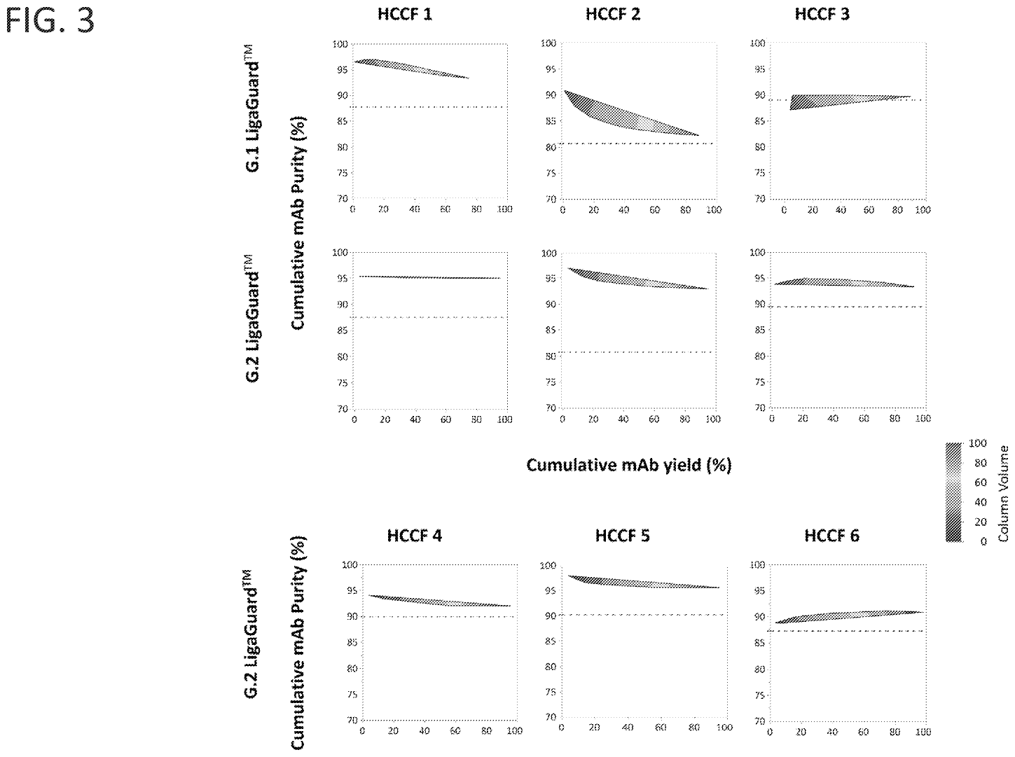

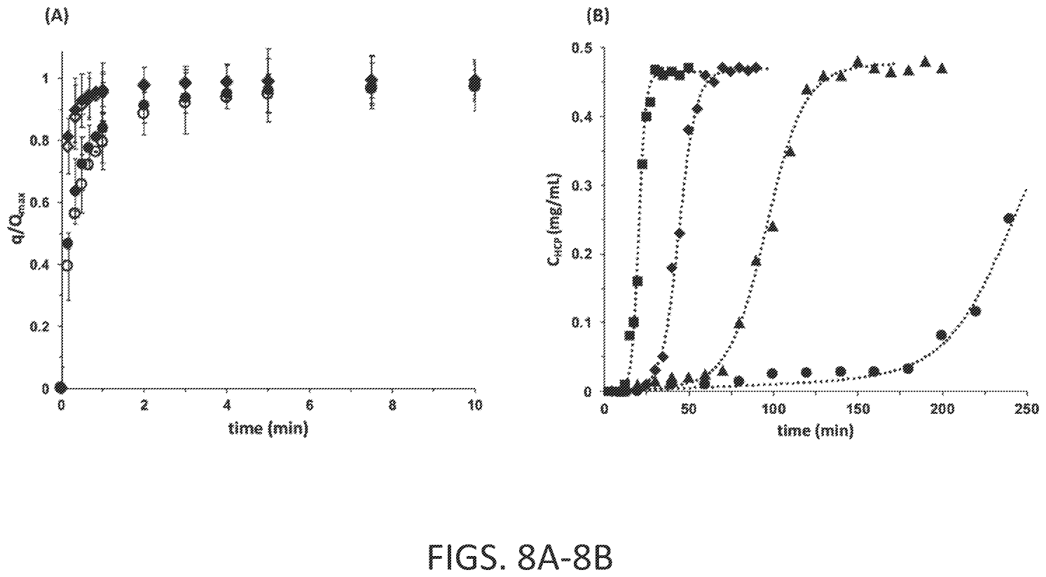

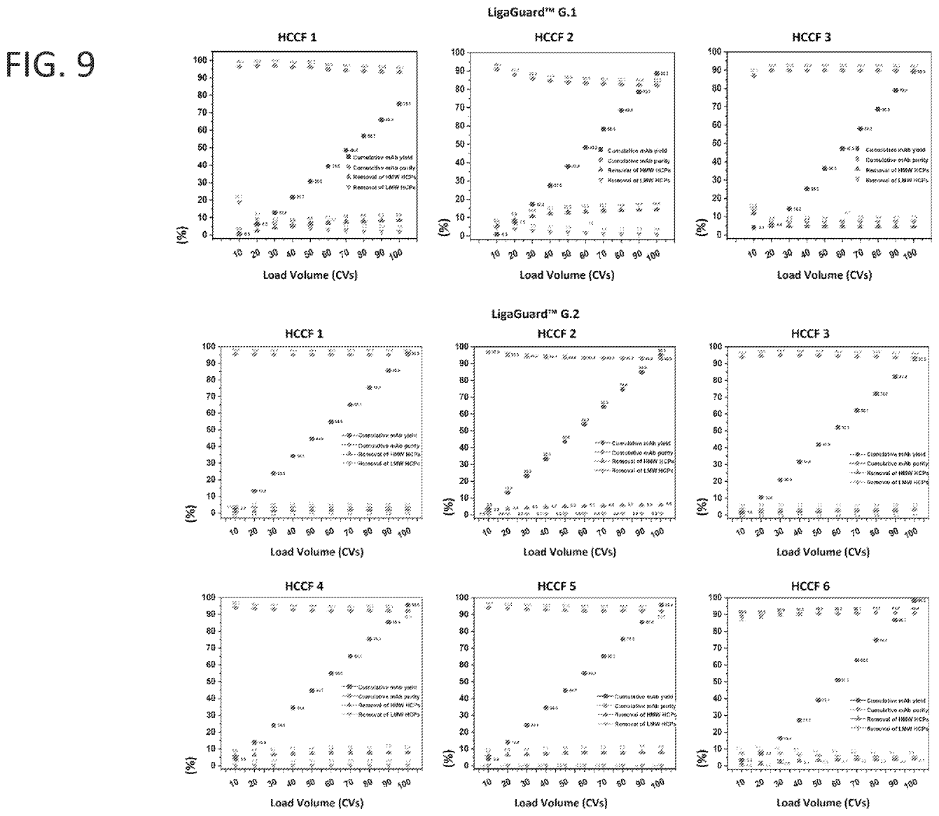

In FIG. 3, contour plots of mAb yield and mAb purity (monomer) are shown as a function of loaded CHO cell culture harvest (0-100 CVs; CV: column volume; note 1: these values are relative to the residence time of 1 min; note 2: the profiles of fractional and cumulative values of mAb yield and purity as functions of loaded volume obtained by loading the CHO cell culture harvests on G.2 LigaGuard™ resins are reported in FIG. 9). These plots help visualize the superior purification power and robustness of G.2 LigaGuard™ resin. At a first glance, the G.2 resin consistently provides higher purity across the entire spectrum of loading volume, and therefore yield, for all tested HCCFs. Secondly, it was observed that the purity of mAb products decreased consistently with the volume of harvest loaded on G.1 resin. This is possibly due to the breakthrough of HCPs with increasing HCCF load, which typically occurs as the load increases and might lead to ligand binding competition between mAb and HCPs, thus lowering the effective HCP binding capacity. A dependence of mAb product quality on loading (i.e., the volumetric ratio of load vs. ligand availability on the chromatographic resin) is expected to a degree when using mixed-mode adsorbents, possibly because of the contact time between HCP species and available ligands. This relationship is a complex function of the HCP profile in the liquid phase and the degree of saturation of the ligands, both of which evolve as the feedstock flows through the adsorbent.

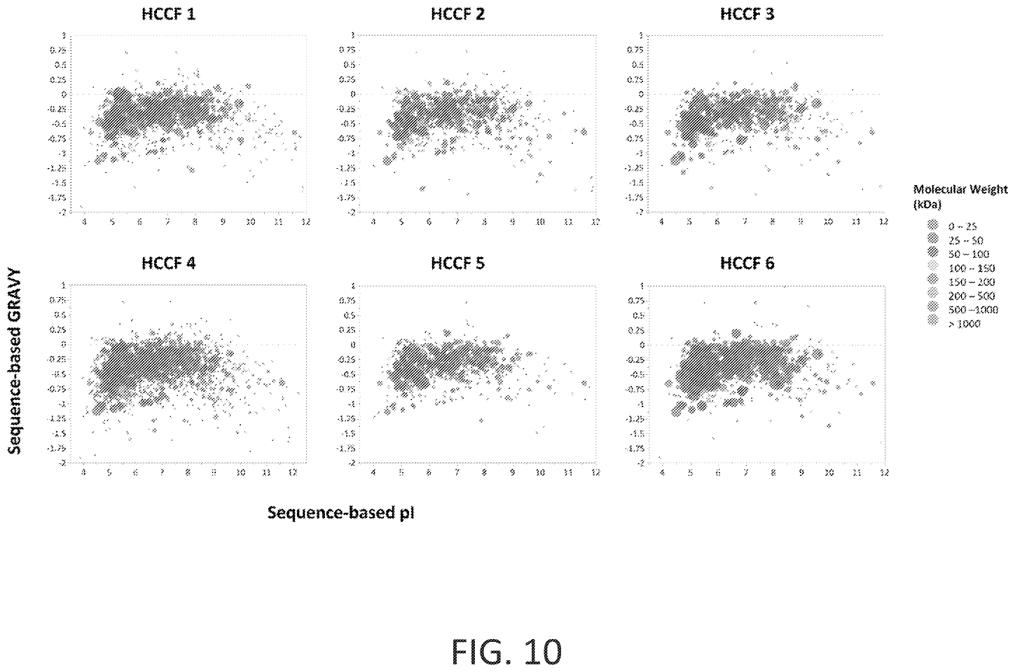

Conversely, the G.2 resin, owing to its higher binding capacity and selectivity, was observed to provide a constant value of mAb purity across the entire range of loading, and therefore cumulative yield. This observation is consistent with the results described in FIG. 3, where other HCCFs were considered and tested with this adsorbent. As detailed in Table 7, the industrial harvests employed in this study differed by cell line, antibody subclass and titer, and significantly, in the titer and properties of HCPs (see the proteomic profiles of the harvests in FIG. 10). High mAb recovery and purity across a broad range of feedstocks and loading volumes denotes strong robustness of the G.2 LigaGuard™ resin, a highly desirable trait in a purification tool, given the diversity of molecular design frameworks, and expression systems and conditions adopted by different biopharmaceutical companies worldwide.

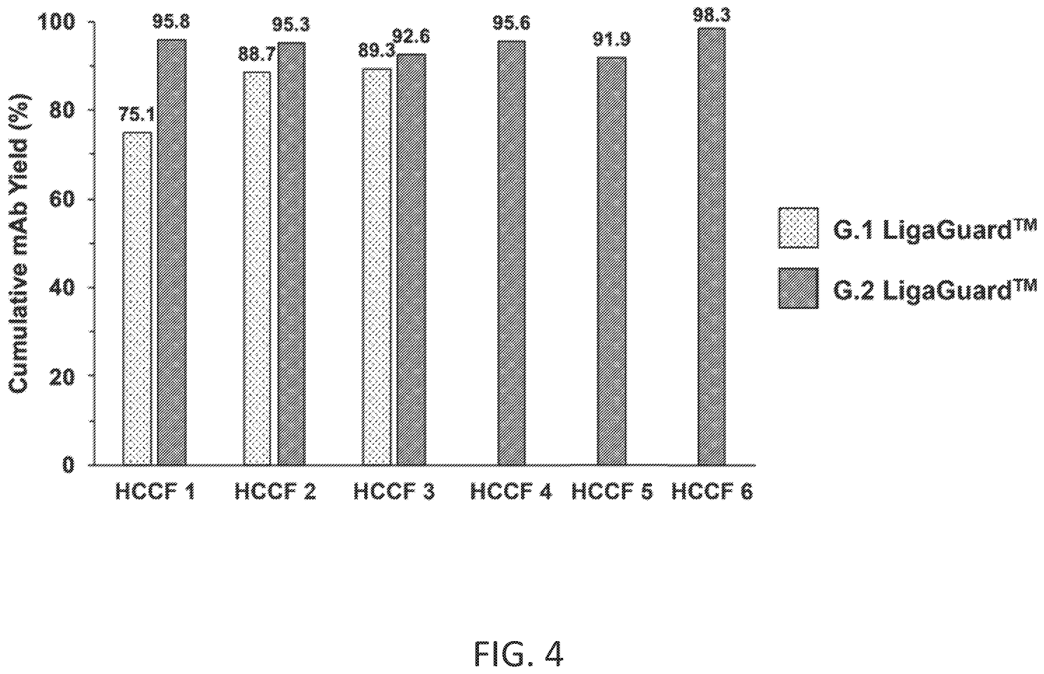

The values of cumulative yield obtained under optimal loading conditions on G.1 and G.2 LigaGuard™ resins are compared in FIG. 4. As noted above, G.2 resin afforded a significantly higher yield of mAb product across all feedstocks compared, namely mAb1, mAb2, and mAb3 harvests, for which a range of 3%-20% difference in yield was observed between G.1 and G.2 resins. Notably, the observation that all values of yield were consistently greater than 90% (as seen with the other tested HCCFs in FIG. 4) when using G.2 resin supports the integration of this technology as an HCP scrubbing step prior to Protein A loading within the current mAb purification platform or next-generation “straight-through” purification processes.

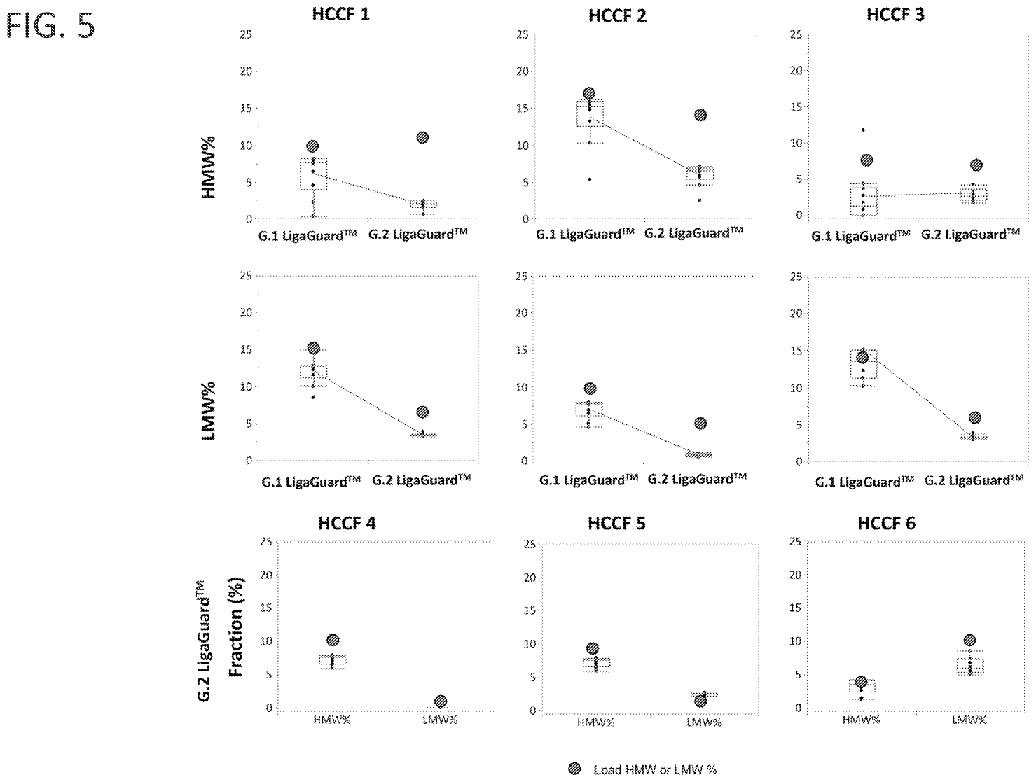

Finally, FIG. 5 summarizes the presence and clearance of impurities distinguished by molecular weight-high molecular weight species (HMW, encompassing the >150 kDa range; includes heavy HCPs and various aggregates formed by mAb and HCPs) and low molecular weight species (LMW, encompassing the 10⁻¹⁵⁰ kDa MW range; includes most of HCPs and mAb fragments)—based on the size exclusion chromatography (SEC) analysis of the harvests and effluents obtained with G.1 and G.2 LigaGuard™ resins.

The various HCCFs tested differed substantially in their impurity profiles-LMW and HMW species, which ranged anywhere between 1%-20% in the feedstocks. The superior purification activity of G.2 resin over its G.1 cognate is well reflected by the clearance of HMW and LMW species. Specifically, the loss of purification power observed with G.1 resin at higher loading ratios translated in both higher means and a larger range of observed values of impurities. Conversely, impurity clearance activity maintained by G.2 resins throughout the entire loading resulted in more consistent product profiles, marked by box plots that are both narrow and markedly separate from the point representing the LMW and HMW composition in the feedstocks (blue). Notably, these data suggest that, by targeting HCPs, the G.2 resin accomplishes the removal of both process- and product-related proteinaceous impurities, including mAb aggregates whose formation is mediated by HCPs, which are ultimately displayed on the surface of the aggregated protein particle.

### Example 3

Clearance of Host Cell Proteins: global and species-specific results. For decades, ELISAs have been considered the global standard for the quantification of titer and HCP clearance in bioprocess streams, and validation of therapeutic mAb production batches continues to rely on ELISA kits certifying that residual HCP impurities are below FDA-imposed limits. Lately, however, prevalence of mass spectrometry as an advanced analytical technique for protein identification and even more recently, quantification, has shown that mAb formulations with acceptable global level of impurities can contain amounts of individual HR-HCPs that pose a threat to patient health due to their inherent immunogenicity or ability to degrade the mAb product during storage. In this context, a growing body of literature is documenting that commercial Protein A and polishing adsorbents struggle to remove HR-HCPs. These “persistent” HR-HCPs have been highlighted on both a process-basis, product batch basis and have been reported to cause delays in clinical trials and process approval, as well as the recall of mAb batches.

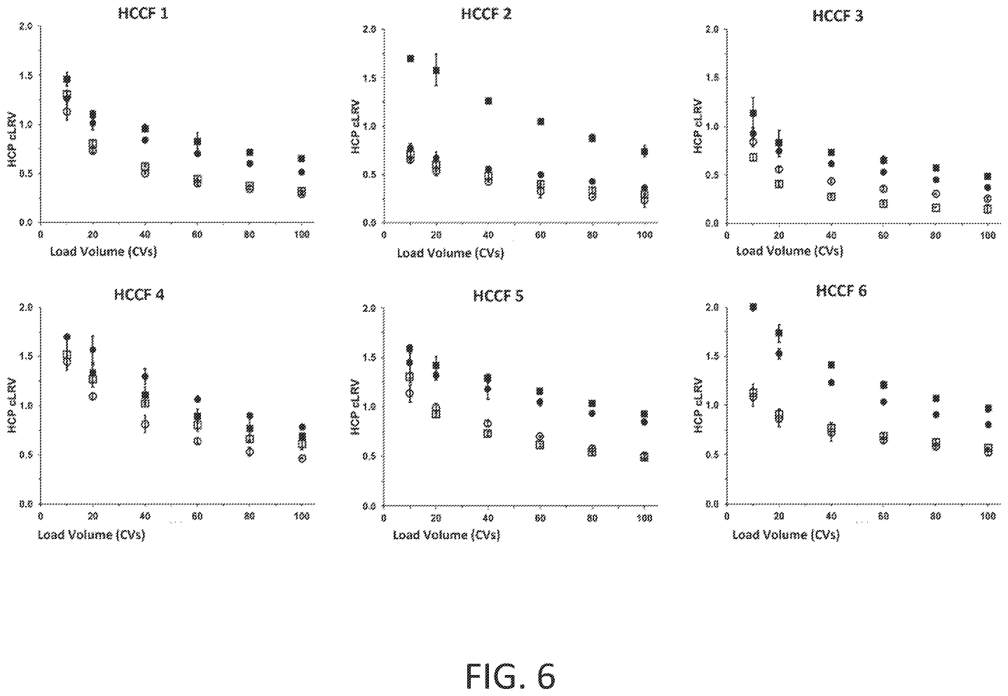

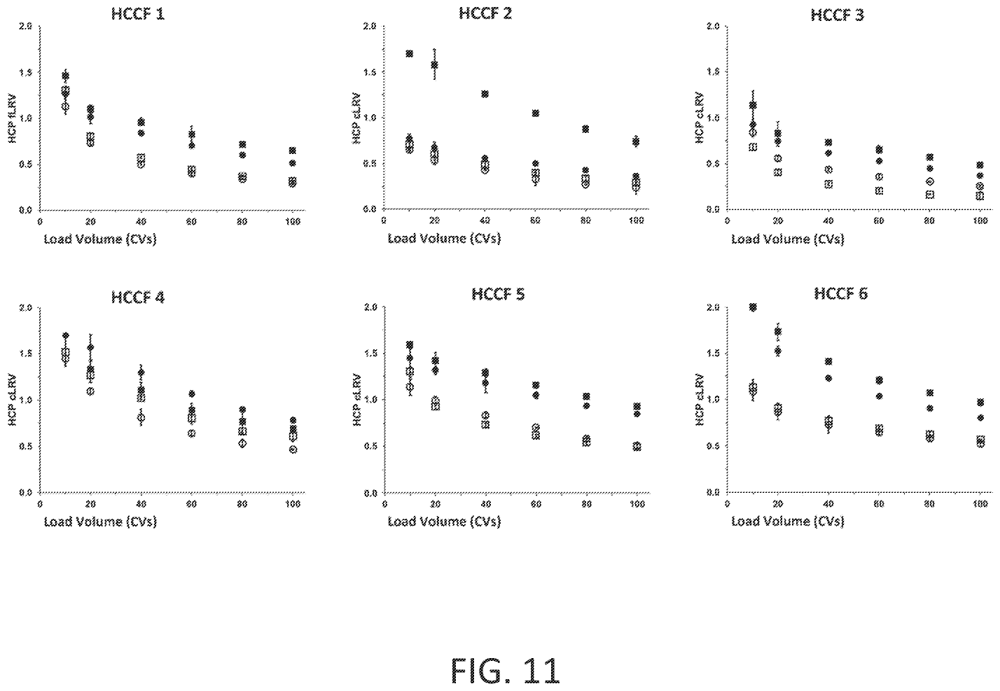

Considering these experiences, the clearance of HCPs by LigaGuard™ resin was evaluate using both global quantification via ELISA and single-protein tracking via proteomic analysis of the flow-through effluents by mass spectrometry (LC-MS/MS). The cumulative values of logarithmic removal (cLRV) of HCPs from the various CHO cell culture harvests are reported in FIG. 6 as functions of the amount of HCCF loaded in CV; (note: the corresponding profiles of fractional LRV (fLRV) as functions of loaded volume are reported in FIG. 11). As noted above, differences in the feedstock properties—namely, HCP titer and composition—and residence times (1 vs. 2 min) resulted into different cLRV profiles. Nonetheless, G.2 LigaGuard™ substantially outperformed its G.1 cognate in HCP removal, accomplishing both remarkable LRVs>2 at low injection volumes and maintaining cLRV>1 throughout the entire loading and flow-through purification process.

Of note is that a higher HCP clearance was consistently observed at the residence time of 1 min. This can possibly be explained by the dynamics of ligand binding between the mAb product and the HCP impurities and competition between them. While the latter is favored both kinetically (k_{on,HCP}>k_{on,mAb}) and thermodynamically (K_{D,HCPs}<K_{D,mAb}), increasing the contact time of a stream at high mAb titer (between 5- to 25-fold higher than the HCP titer) may trigger the displacement of HCPs and binding of mAb, thus lowering both product yield and purity. At a residence time of 1 minute, it was observed that this phenomenon is avoided, hence becoming conducive to both higher product throughput and quality.

As the HCP binding peptide ligands become progressively saturated, their ability to capture individual HCPs or HCP classes may decrease. Accordingly, as the loading progresses, monitoring the effluents becomes necessary towards tracking the breakthrough of specific HCPs that would pose a threat to product quality and patients' safety. In this regard, a conspicuous body of literature has identified and characterized the role of CHO HCPs that persist through the purification pipeline by coeluting with the mAb product from the Protein A resin and escape the polishing adsorbents, and are highly immunogenic or degrade the mAb product during storage.

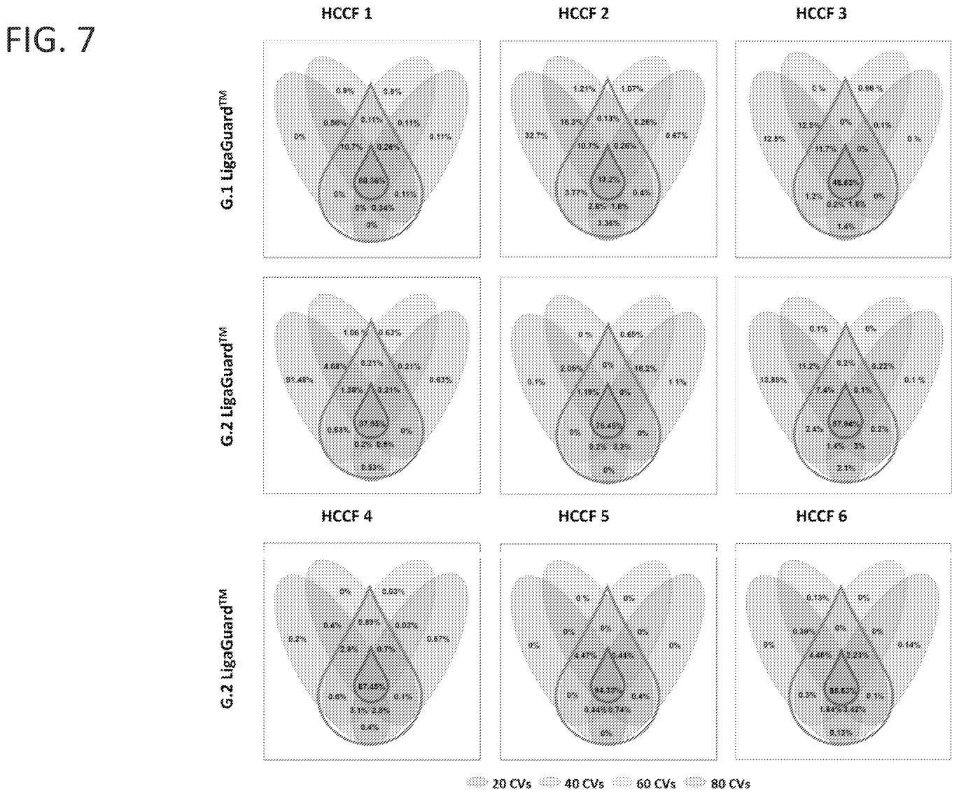

To document the ability of LigaGuard™ resins to target and effectively clear these persistent and “high risk” (HR) HCPs, proteomics analysis of the flow-through fractions was performed via LC-MS/MS analysis. As detailed in prior work, CHO HCPs were identified and quantitatively tracked via intensity-based absolute quantification (iBAQ). Significantly “captured” HCPs were defined as proteins that were either—(i) identified in the HCCF but not in the effluent (note: an HCP is “identified” when the sum of the spectral counts of its fragments is >4) or (ii) are compared to be statistically significantly lower than in the HCCF, computed by performing ANOVA. The numbers of HCPs captured by G.1 and G.2 LigaGuard™ resins either uniquely at specific loading (CVs) or completely—throughout the run have been represented and expressed as a % fraction of the total number of captured HCPs throughout the run in FIG. 7.

While these values do not portray the mass or concentration of HCPs removed and are therefore not directly comparable to LRVs, they provide a measure of HCP capture coverage achieved with different HCCFs by LigaGuard™ resins. Comprehensive coverage in targeting the HCPs is denoted by the overlap of bound HCPs across different fractions—represented by droplet boundaries (red and blue) in FIG. 7. Across compared groups (FIG. 7, lines 1 and 2), HCCFs 2 and 3 showed a tremendous improvement in the ability to capture a wide variety of HCPs consistently throughout the flow-through experiment, while HCCF 1 showed very high clearance of HCPs within the first 30 CV of load. On the other hand, all the other HCCFs evaluated with G.2 (FIG. 7, line 3) corroborate a similar observation of very high comprehensive coverage.

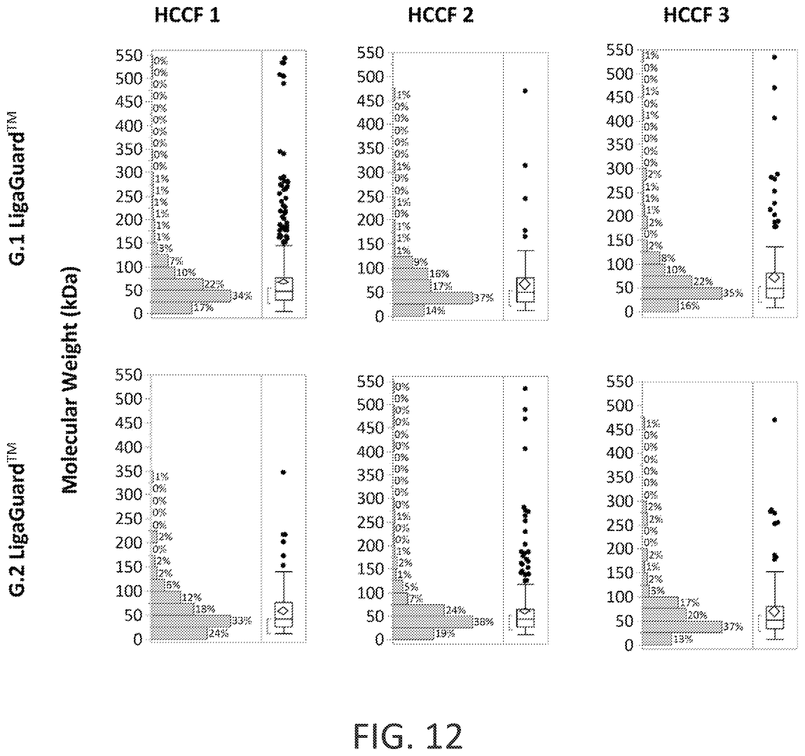

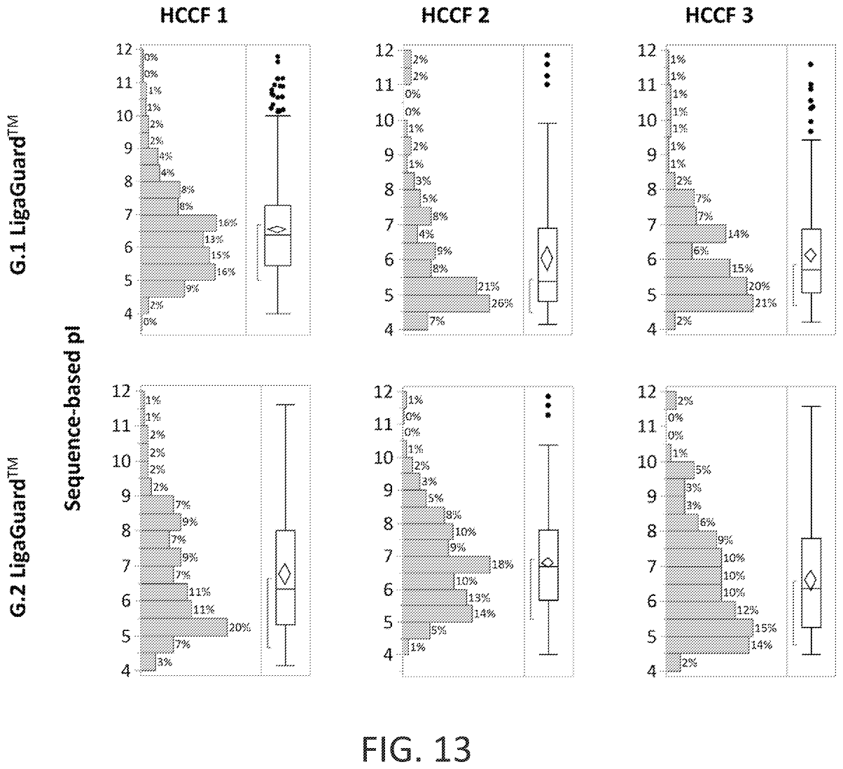

The commonly bound HCPs were then surveyed, including those in the blue droplet boundaries, to identify notable differences in the classes of HCPs captured by G.1 and G.2 LigaGuard™ resins. While both adsorbents demonstrated the ability to capture HCPs differing greatly by molecular weight (16-650 kDa, (FIG. 12) and physicochemical properties (i.e., isoelectric point and grand average of hydropathy—GRAVY, (FIGS. 10 and 13) their HCP capturing activity continued to evolve as the harvests were loaded. For example, there were species that were captured within 0-20 CVs and 80-100 CVs of injected harvest, but not in between (21-79 CVs); furthermore, some species that were uniquely captured between 21-40 CVs or 41-60 CVs. Finally, except for HCCF3, the progression of HCP capture coverage at difference load volumes is rather different between G.1 and G.2 resins. Similarly, while G.2 resin featured a high binding robustness, consistently capturing 40-95% of HCPs (blue droplet boundaries in FIG. 7), notable differences in HCP capture coverage by G.2 resin across the various harvests were nonetheless registered. These phenomena may have different causes-chiefly, the diversity and complexity of HCPs in different HCCFs, but also HCPs competing for ligand binding as the latter become progressively saturated at higher loading. It is also to be noted that there exists an effect of residence time (1 minute) on these results between groups. The interconnected effects of formation and disruption of HCP:HCP interactions and HCP:mAb interactions on the affinity surface, especially in solution, cannot be easily deconvoluted. Therefore, the results above should be viewed phenomenologically and not mechanistically.

The most important conclusion obtained from the proteomics analysis of the effluents is the clearance of “persistent”, “high-risk” CHO HCPs identified from the various harvests. A list of HCPs commonly identified in industrial bioprocesses and the ability of G.1 and G.2 LigaGuard™ resins to clear them is summarized in Table 2 and comprehensively reported in Table 3. As HR-HCPs from comprehensively bound protein groups were chosen to perform this comparison, it is to be noted that uncolored cells either relate to proteins that were undetected in the respective load samples or have been cleared as a function of CV as shown in FIG. 7. In case the HR-HCP absolute concentration is very low, it is possible that after being captured at a specific time point, the protein is not detected in rest of the flow-through fractions.

As mentioned above, while demonstrating the value in the “flow through affinity chromatography” paradigm, the G.1 precursor was inadequate in capturing some of the highly problematic HCPs. Conversely, G.2 LigaGuard™ resin successfully and consistently cleared these species. It is important to note that these HCPs not only pose a risk to product safety due to their high immunogenicity (risk class 3) but may also possess the ability to degrade the mAb product or the excipients that ensure its stability during storage (risk class 2). With many of these HCPs being proteolytically active-Serine Proteases, Cathepsins, Metalloproteases, Lipases etc.—not only the mAb product, but also the Protein A ligand can be degraded upon prolonged exposure, thus releasing dangerous fragments, and losing its purification efficiency. The latter results in discrepancies between the declared lifetime of Protein A media, typically up to 200 cycles of alkaline regeneration, and their actual lifetime in bioprocessing. These results demonstrated that LigaGuard™ resins either completely, or at least partially, yet substantially clear these HR-HCPs and can effectively prevent such deterring issues from manifesting as a risk to the patients and a burden to the industry.

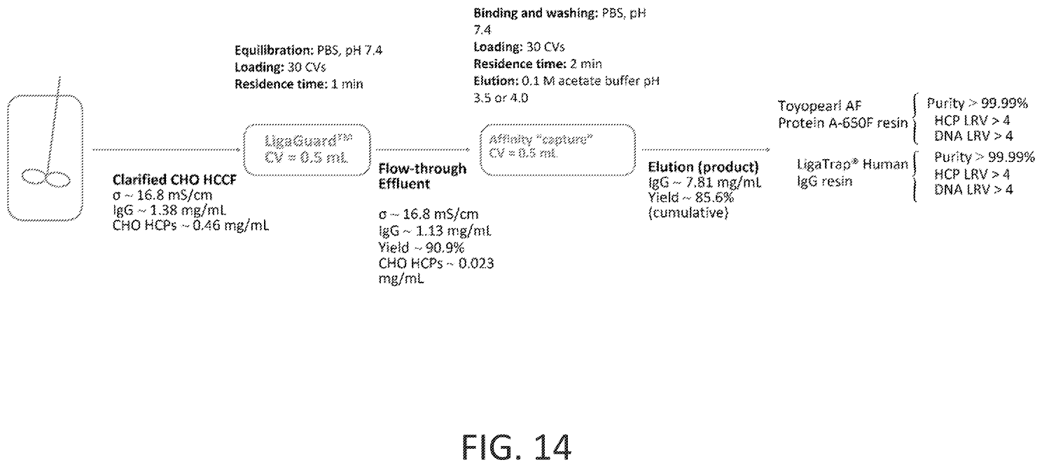

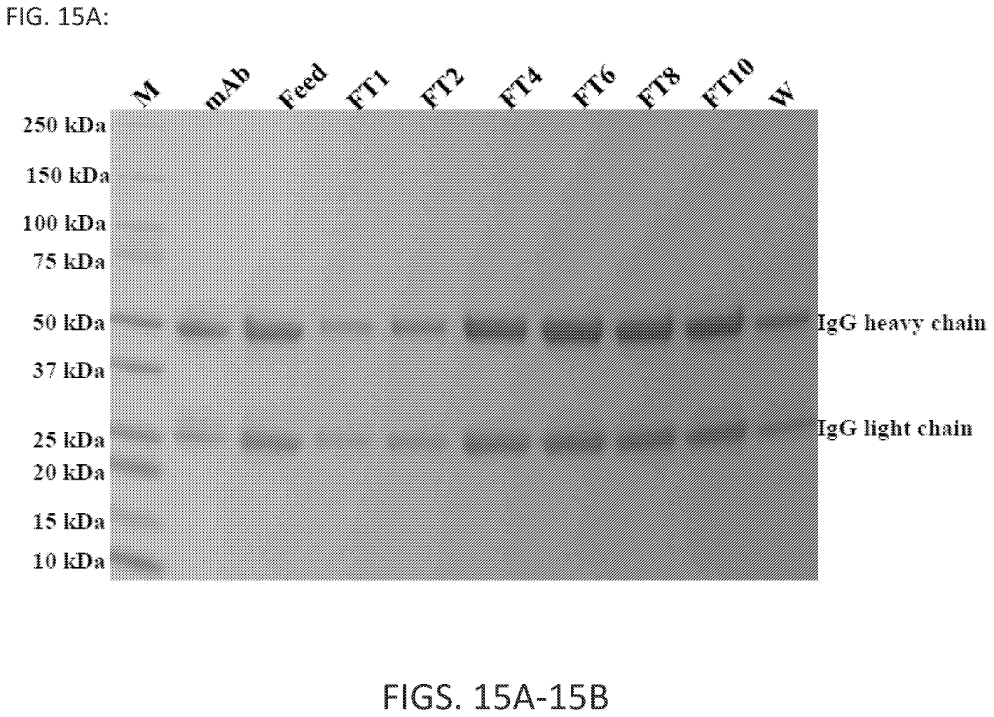

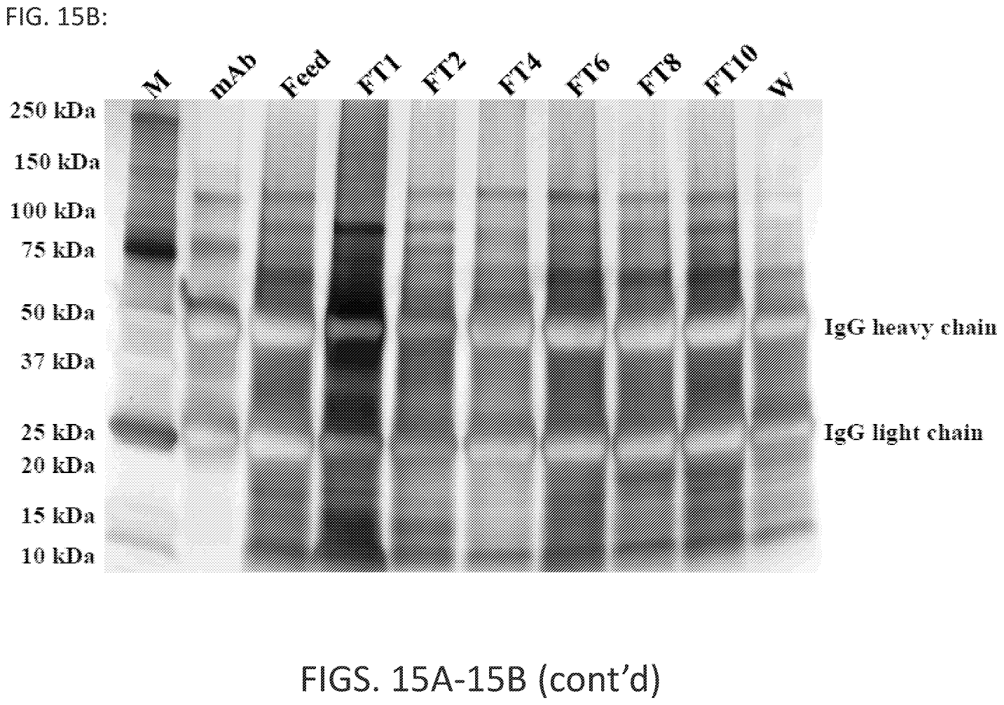

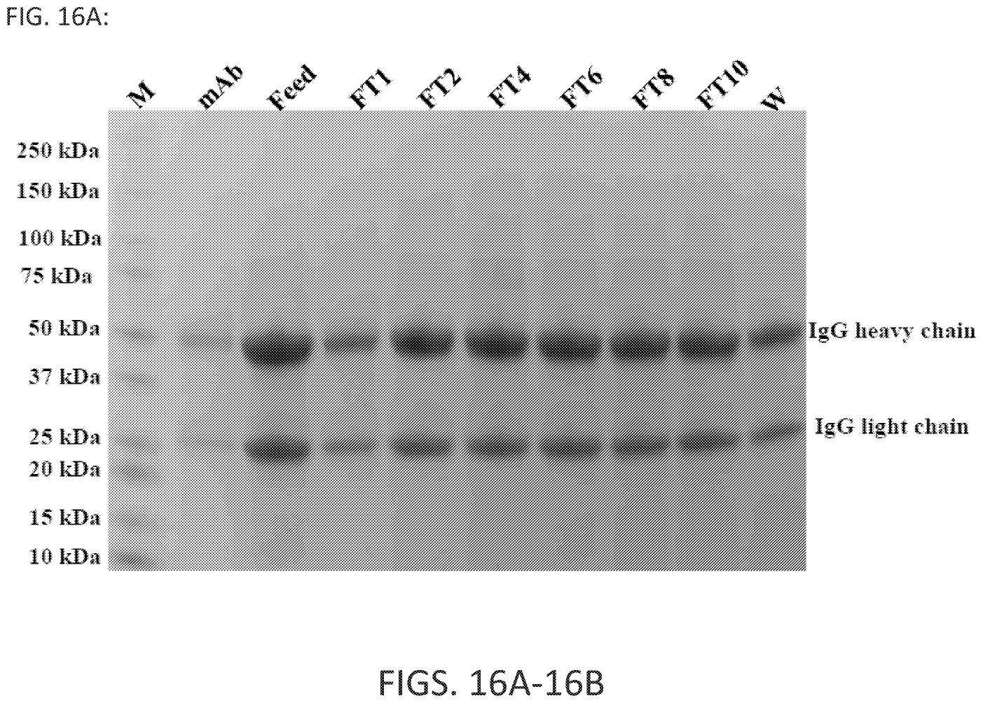

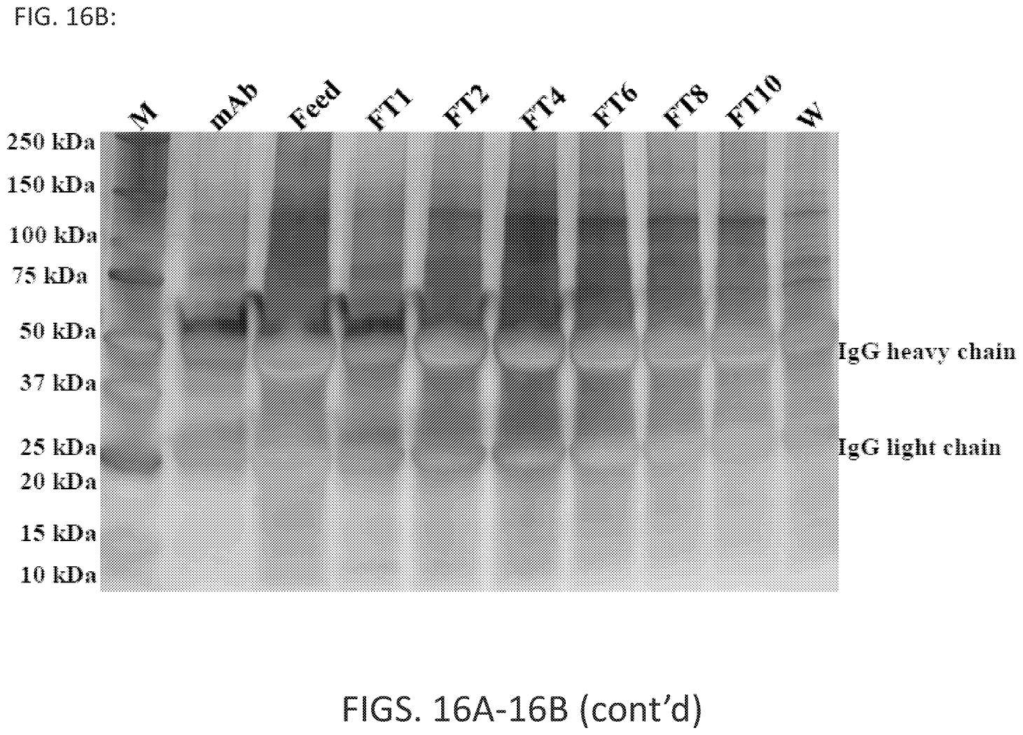

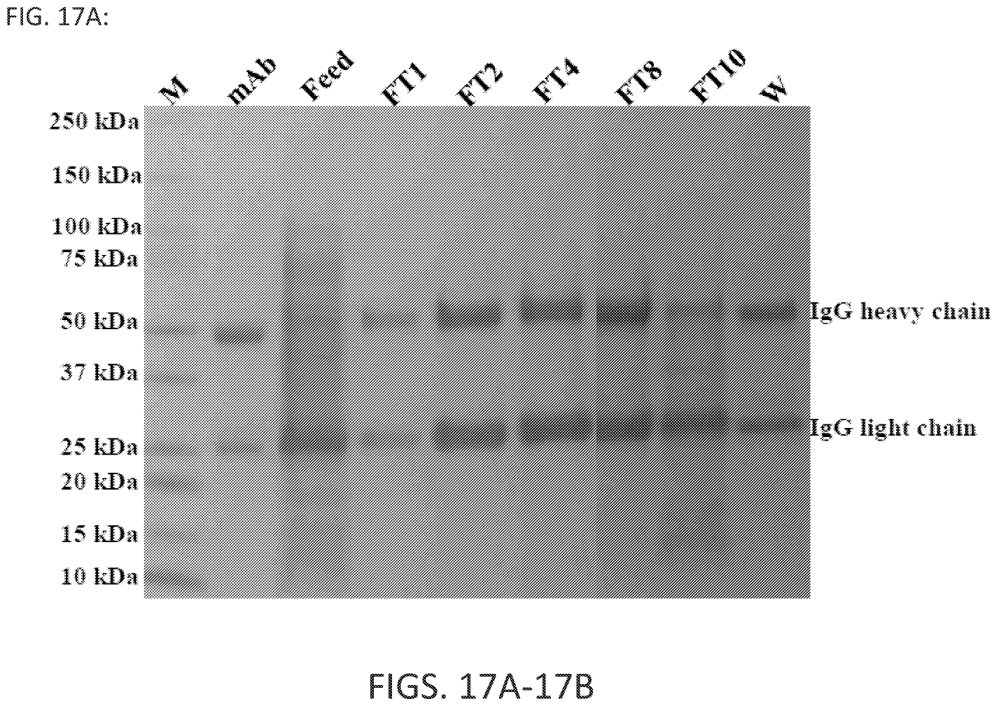

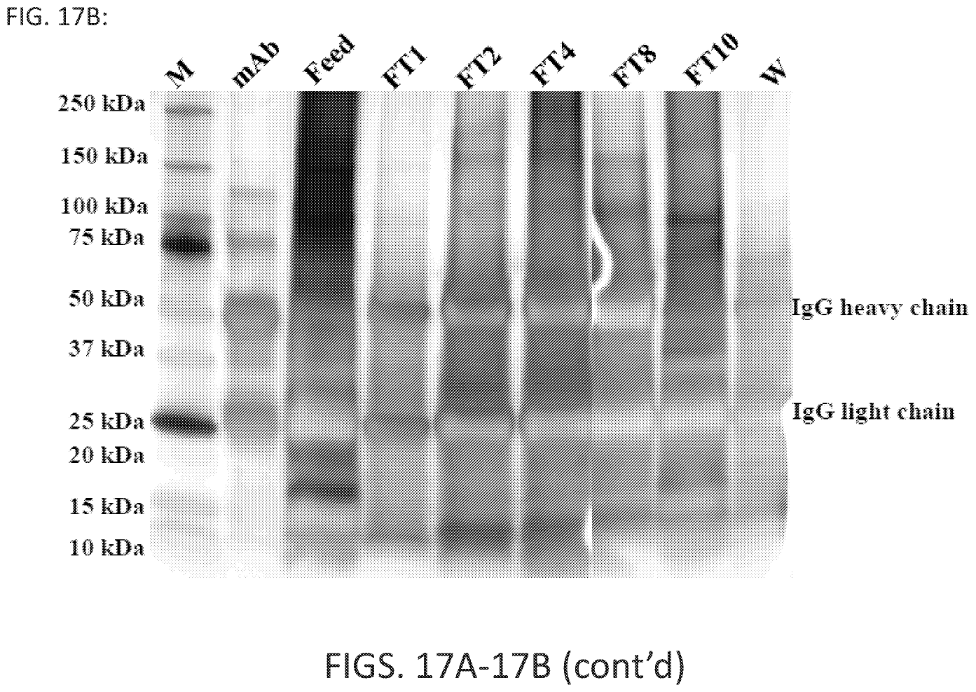

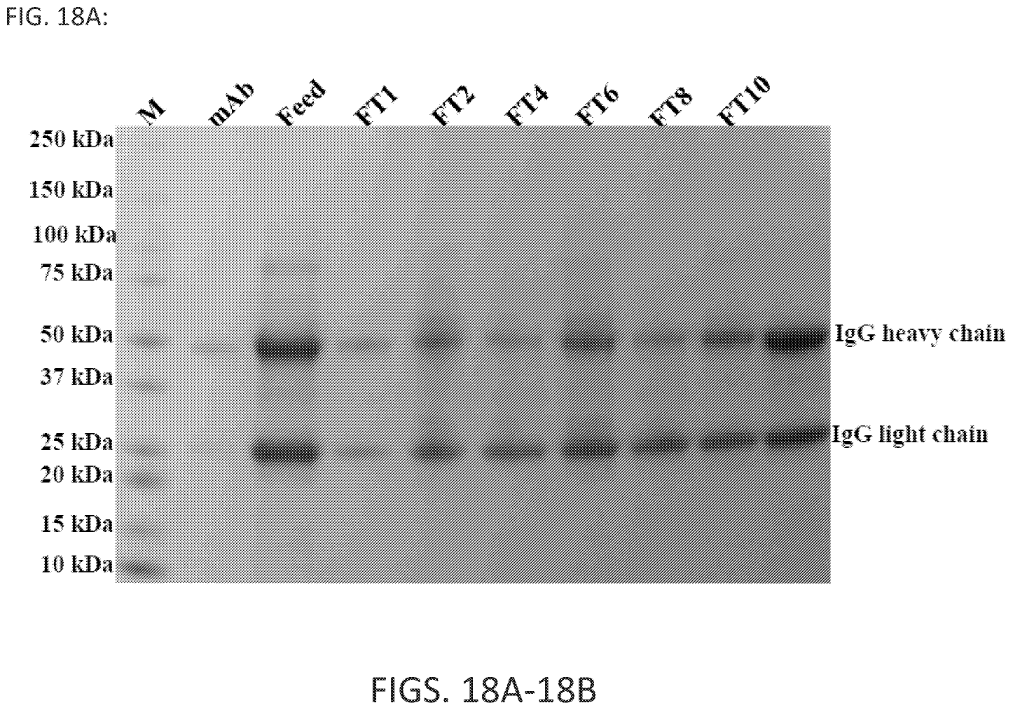

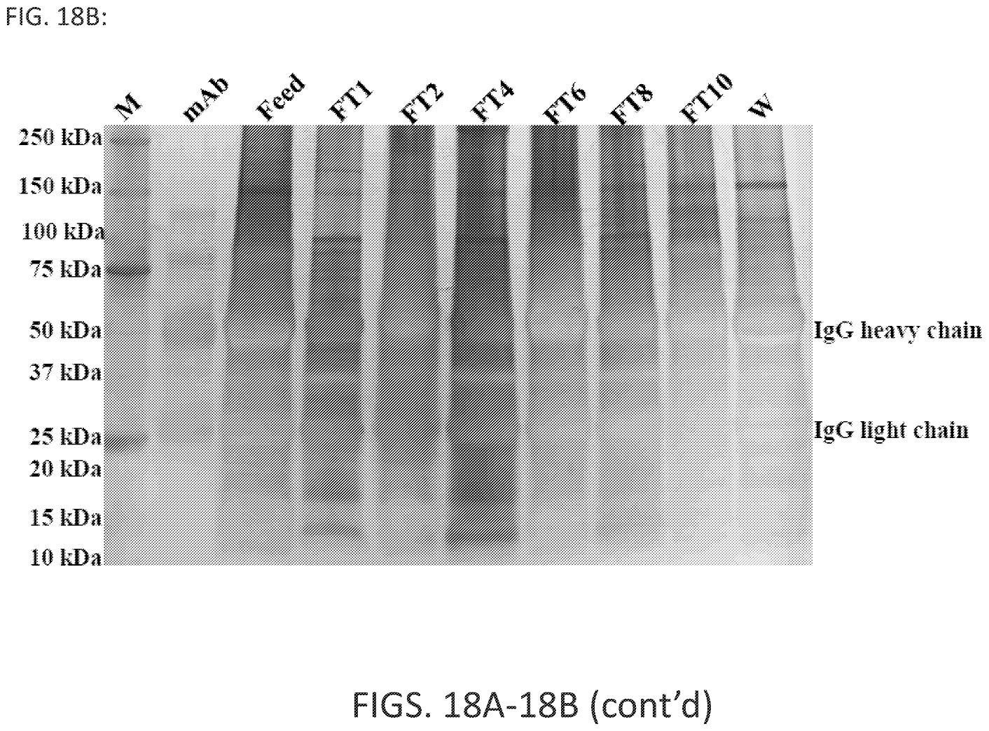

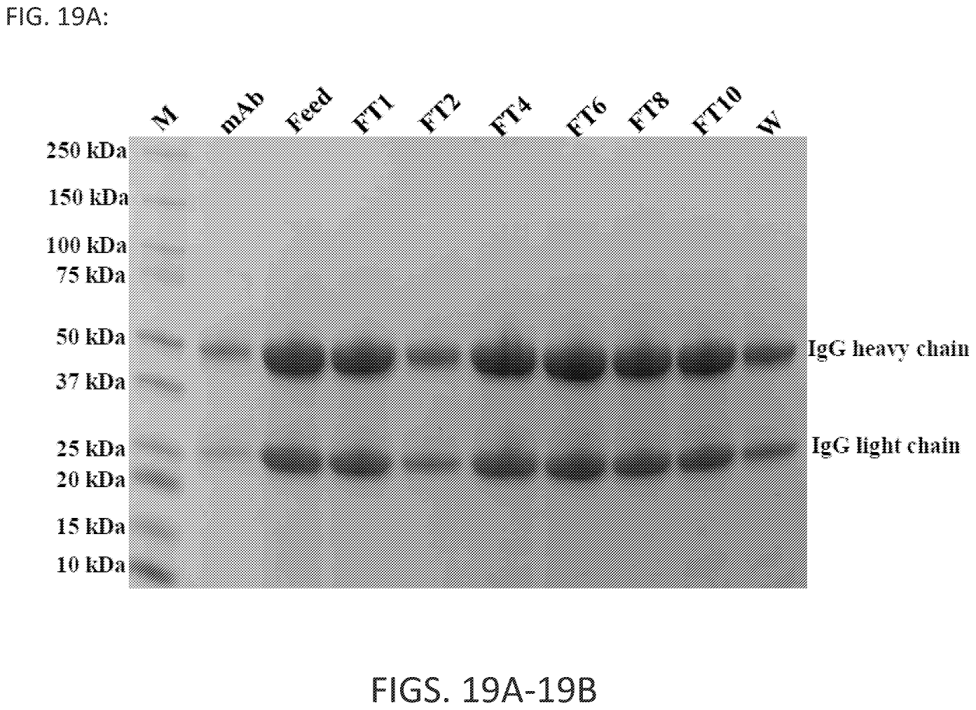

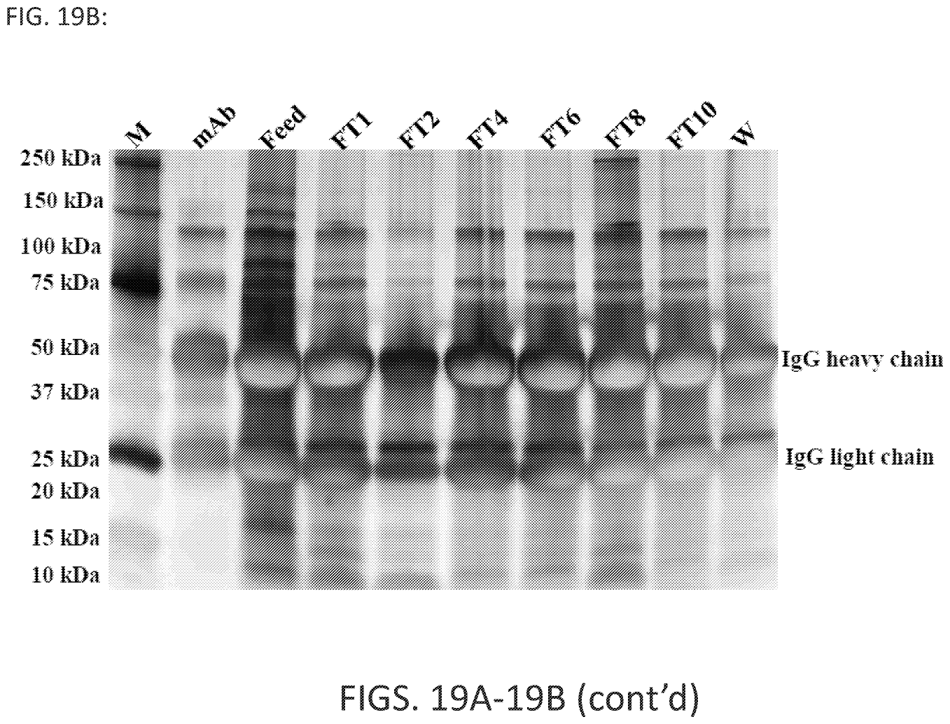

As a part of the final evaluation, the effluent from the G.2 LigaGuard™ resin was used to feed an affinity adsorbent-either a Protein A-based Toyopearl AF-rProtein A-650F resin or LigaTrap® Human IgG resin packed in 0.1 mL chromatography column (FIG. 14). Following binding, the column was washed using PBS at pH 7.4 and eluted from Toyopearl AF-rProtein A-650F and LigaTrap® Human IgG resins using 0.1 M acetic acid at pH 3.5 and 4.0, respectively. Both resins were regenerated using 0.1 M glycine buffer at pH 2.5 and cleaned using 0.5 M aqueous NaOH. The eluted fractions were analyzed as described herein to determine mAb yield and to determine HCP LRV, which was estimated to afford a 4-log clearance of HCPs.

TABLE 2
Selected list of persistent, high-risk HCPs and their corresponding risk class removed by G.1 and
G.2 LigaGuard ™ resin via flow-through affinity chromatography from six industrial harvested
cell culture fluids (HCCF); risk group 1 comprises HCPs that co-elute with and can degrade the mAb
product, while risk group 2 comprises highly immunogenic HCPs. The full list is reported in Table 3.

| | | mAb | mAb | mAb | mAb | mAb | mAb | mAb | mAb | mAb |
|---|---|---|---|---|---|---|---|---|---|---|
| | Risk | 1 | 2 | 3 | 1 | 2 | 3 | 4 | 5 | 6 |
| Description | Group | G.1 LigaTrap ™ | | | G.2 LigaTrap ™ | | | | | |
| 60S ribosomal protein isoforms | 1 | [symbol] | [symbol] | [symbol] | [symbol] | [symbol] | [symbol] | [symbol] | | |
| Alpha-enolase isoform X2 | 1 | [symbol] | [symbol] | [symbol] | [symbol] | [symbol] | [symbol] | | [symbol] | [symbol] |
| Annexin A1 | 2 | [symbol] | [symbol] | [symbol] | [symbol] | [symbol] | [symbol] | [symbol] | [symbol] | [symbol] |
| Carboxypeptidase D X2 | 1 | [symbol] | [symbol] | [symbol] | [symbol] | [symbol] | [symbol] | | | [symbol] |
| Cathepsin B | 1 | [symbol] | [symbol] | [symbol] | [symbol] | [symbol] | [symbol] | [symbol] | [symbol] | [symbol] |
| Cathepsin D | 1 | | [symbol] | [symbol] | | [symbol] | [symbol] | [symbol] | [symbol] | [symbol] |
| Cathepsin F isoform X1 | | [symbol] | | | [symbol] | | | [symbol] | | |
| Cathepsin L 1 | 1 | [symbol] | [symbol] | [symbol] | [symbol] | [symbol] | [symbol] | [symbol] | [symbol] | [symbol] |
| Cathepsin Z | 1 | [symbol] | [symbol] | [symbol] | [symbol] | [symbol] | [symbol] | [symbol] | [symbol] | [symbol] |
| Clusterin | 1 | | [symbol] | [symbol] | | [symbol] | [symbol] | [symbol] | [symbol] | [symbol] |
| C-X-C motif chemokine 3 precursor | 2 | [symbol] | | [symbol] | [symbol] | | [symbol] | [symbol] | | [symbol] |
| Endoplasmic reticulum chaperone BiP precursor | 1 | [symbol] | [symbol] | [symbol] | [symbol] | [symbol] | [symbol] | | [symbol] | [symbol] |
| Enolase-phosphatase E1 isoform X1 | 2 | | | [symbol] | | | [symbol] | | | [symbol] |
| Glutathione S-transferase A4 | 2 | [symbol] | [symbol] | [symbol] | [symbol] | [symbol] | [symbol] | [symbol] | [symbol] | [symbol] |
| Glutathione S-transferase Mu 6 | 2 | | [symbol] | [symbol] | | [symbol] | [symbol] | [symbol] | [symbol] | [symbol] |
| Group XV phospholipase A2 isoform X2 | 1 | | | [symbol] | | | [symbol] | [symbol] | | [symbol] |
| Histone H2A type 1-H isoform X1 | 1 | | [symbol] | | | [symbol] | | [symbol] | [symbol] | [symbol] |
| Histone H3 isoform X2 | 1 | [symbol] | [symbol] | [symbol] | [symbol] | [symbol] | [symbol] | [symbol] | [symbol] | [symbol] |
| Lipoprotein lipase isoform X1 | 1 | [symbol] | [symbol] | [symbol] | [symbol] | [symbol] | [symbol] | [symbol] | [symbol] | [symbol] |
| Peptidyl-prolyl cis-trans isomerase B | 1 | | [symbol] | [symbol] | | [symbol] | [symbol] | [symbol] | [symbol] | [symbol] |
| Peptidyl-prolyl cis-trans isomerase C isoform X2 | 1 | [symbol] | [symbol] | [symbol] | [symbol] | [symbol] | [symbol] | [symbol] | [symbol] | [symbol] |
| Protein S100-A11 | 2 | [symbol] | [symbol] | [symbol] | [symbol] | [symbol] | [symbol] | [symbol] | [symbol] | [symbol] |
| Protein S100-A6 isoform X1 | 2 | [symbol] | [symbol] | [symbol] | [symbol] | [symbol] | [symbol] | [symbol] | [symbol] | [symbol] |
| Phospholipase B-like 2 | 2 | | [symbol] | [symbol] | | [symbol] | [symbol] | [symbol] | [symbol] | [symbol] |
| Pyruvate kinase PKM X1 | 2 | [symbol] | | [symbol] | [symbol] | | [symbol] | [symbol] | | [symbol] |
| Serine protease HTRA2, mitochondrial | 1 | | | [symbol] | | | | [symbol] | [symbol] | |
| Transforming growth factor beta receptor type 3 | 2 | [symbol] | [symbol] | | [symbol] | [symbol] | | [symbol] | [symbol] | |
| ubiquitin-60S ribosomal protein L40 isoform X1 | 1 | | | [symbol] | | | [symbol] | [symbol] | [symbol] | [symbol] |

[symbol]

indicates data missing or illegible when filed

TABLE 3
Complete list of persistent, high-risk HCPs and their corresponding risk class removed by G.1 and G.2 LigaGuard ™ resin
via flow-through affinity chromatography from six industrial harvested cell culture fluids (HCCF); risk group 1 comprises
HCPs that co-elute with and can degrade the mAb product, while risk group 2 comprises highly immunogenic HCPs.

| | | mAb | mAb | mAb | mAb | mAb | mAb | mAb | mAb | mAb |
|---|---|---|---|---|---|---|---|---|---|---|
| | Risk | 1 | 2 | 3 | 1 | 2 | 3 | 4 | 5 | 6 |
| Description | Group | G.1 LigaTrap ™ | | | G.2 LigaTrap ™ | | | | | |
| 60S acidic ribosomal protein P0 isoform X1 | 1 | | [symbol] | [symbol] | | [symbol] | [symbol] | [symbol] | [symbol] | [symbol] |
| 60S acidic ribosomal protein P2 isoform X1 | 1 | [symbol] | | [symbol] | [symbol] | | [symbol] | [symbol] | [symbol] | |
| 60S ribosomal protein L11-like | 1 | [symbol] | [symbol] | [symbol] | [symbol] | [symbol] | [symbol] | [symbol] | [symbol] | [symbol] |
| 60S ribosomal protein L15 | 1 | [symbol] | | | [symbol] | | | [symbol] | [symbol] | |
| 60S ribosomal protein L18a isoform X2 | 1 | [symbol] | [symbol] | [symbol] | [symbol] | [symbol] | [symbol] | [symbol] | | |
| 60S ribosomal protein L7a isoform X2 | 1 | [symbol] | | [symbol] | [symbol] | | [symbol] | [symbol] | | |
| alpha-enolase isoform X2 | 1 | [symbol] | [symbol] | [symbol] | [symbol] | [symbol] | [symbol] | | [symbol] | [symbol] |
| annexin A1 | 2 | [symbol] | [symbol] | [symbol] | [symbol] | [symbol] | [symbol] | [symbol] | [symbol] | [symbol] |
| annexin A2 isoform X2 | 2 | [symbol] | [symbol] | [symbol] | [symbol] | [symbol] | [symbol] | [symbol] | | |
| annexin A4 | 2 | | | [symbol] | | | [symbol] | | | |
| annexin A5 | 2 | | [symbol] | [symbol] | | [symbol] | [symbol] | | | [symbol] |
| annexin A6 isoform X2 | 2 | | [symbol] | | | [symbol] | | | | |
| annexin A7 isoform X1 | 2 | | [symbol] | [symbol] | | [symbol] | [symbol] | | | [symbol] |
| Carboxypeptidase D isoform X2 | 1 | [symbol] | [symbol] | [symbol] | [symbol] | [symbol] | [symbol] | | | [symbol] |
| cathepsin B | 1 | [symbol] | [symbol] | [symbol] | [symbol] | [symbol] | [symbol] | [symbol] | [symbol] | [symbol] |
| cathepsin D | 1 | | [symbol] | [symbol] | | [symbol] | [symbol] | [symbol] | [symbol] | [symbol] |
| cathepsin F isoform X1 | | [symbol] | | | [symbol] | | | [symbol] | | |
| cathepsin L1 | 1 | [symbol] | [symbol] | [symbol] | [symbol] | [symbol] | [symbol] | [symbol] | [symbol] | [symbol] |
| cathepsin Z | 1 | [symbol] | [symbol] | [symbol] | [symbol] | [symbol] | [symbol] | [symbol] | [symbol] | [symbol] |
| clusterin | 1 | | [symbol] | [symbol] | | [symbol] | [symbol] | [symbol] | [symbol] | [symbol] |
| C-X-C motif chemokine 3 precursor | 2 | [symbol] | | [symbol] | [symbol] | | [symbol] | [symbol] | | [symbol] |
| endoplasmic reticulum chaperone BiP precursor | 1 | [symbol] | [symbol] | [symbol] | [symbol] | [symbol] | [symbol] | | [symbol] | [symbol] |
| enolase-phosphatase E1 isoform X1 | 2 | | | [symbol] | | | [symbol] | | | [symbol] |
| glutathione S-transferase A4 | 2 | [symbol] | [symbol] | [symbol] | [symbol] | [symbol] | [symbol] | [symbol] | [symbol] | [symbol] |
| glutathione S-transferase alpha-3 | 2 | [symbol] | | | [symbol] | | | [symbol] | | [symbol] |
| glutathione S-transferase isoform X1 | 2 | [symbol] | | [symbol] | [symbol] | | [symbol] | | | |
| glutathione S-transferase Mu 1 isoform X1 | 2 | [symbol] | [symbol] | [symbol] | [symbol] | [symbol] | [symbol] | [symbol] | [symbol] | [symbol] |
| glutathione S-transferase Mu 3 | 2 | | | [symbol] | | | [symbol] | [symbol] | [symbol] | |
| glutathione S-transferase Mu 3 isoform X3 | 2 | [symbol] | | | [symbol] | | | [symbol] | [symbol] | |
| glutathione S-transferase Mu 6 | 2 | | [symbol] | [symbol] | | [symbol] | [symbol] | [symbol] | [symbol] | [symbol] |
| glutathione S-transferase Mu 7 isoform X2 | 2 | [symbol] | [symbol] | [symbol] | [symbol] | [symbol] | [symbol] | [symbol] | [symbol] | |
| glutathione S-transferase omega-1 isoform X4 | 2 | [symbol] | | | [symbol] | | | [symbol] | | [symbol] |
| glutathione S-transferase omega-1 isoform X6 | 2 | [symbol] | | [symbol] | [symbol] | | [symbol] | [symbol] | [symbol] | |
| glutathione S-transferase P 1 | 2 | [symbol] | [symbol] | [symbol] | [symbol] | [symbol] | [symbol] | [symbol] | [symbol] | [symbol] |
| glutathione S-transferase P 2 isoform X2 | 2 | [symbol] | | | [symbol] | | | | [symbol] | [symbol] |
| glutathione S-transferase Y1 isoform X2 | 2 | | [symbol] | [symbol] | | [symbol] | [symbol] | | [symbol] | [symbol] |
| glutathione synthetase isoform X1 | 2 | | [symbol] | [symbol] | | [symbol] | [symbol] | [symbol] | [symbol] | |
| group XV phospholipase A2 isoform X2 | 1 | | | [symbol] | | | [symbol] | [symbol] | | [symbol] |
| heat shock protein HSP 90-alpha | 1 | [symbol] | [symbol] | [symbol] | [symbol] | [symbol] | [symbol] | | [symbol] | |
| heat shock protein HSP 90-beta isoform X11 | 1 | | [symbol] | [symbol] | | [symbol] | [symbol] | | [symbol] | [symbol] |
| histone H2A type 1-H isoform X1 | 1 | | [symbol] | | | [symbol] | | [symbol] | [symbol] | [symbol] |
| histone H2B type 2-F | 1 | [symbol] | [symbol] | [symbol] | [symbol] | [symbol] | [symbol] | | [symbol] | [symbol] |
| histone H3 isoform X2 | 1 | [symbol] | [symbol] | [symbol] | [symbol] | [symbol] | [symbol] | [symbol] | [symbol] | [symbol] |
| histone H3.3 isoform X2 | | | [symbol] | | | [symbol] | | | | |
| legumain | 1 | | [symbol] | [symbol] | | [symbol] | [symbol] | [symbol] | [symbol] | [symbol] |
| lipoprotein lipase isoform X1 | 1 | [symbol] | [symbol] | [symbol] | [symbol] | [symbol] | [symbol] | [symbol] | [symbol] | [symbol] |
| lysosomal acid lipase/cholesteryl ester hydrolase | 1 | [symbol] | | [symbol] | [symbol] | | [symbol] | [symbol] | | |
| matrix metalloproteinase-19 | 1 | | [symbol] | [symbol] | | [symbol] | [symbol] | [symbol] | [symbol] | [symbol] |
| matrix metalloproteinase 9 | 1 | [symbol] | | [symbol] | [symbol] | | [symbol] | [symbol] | | [symbol] |
| nidogen-1 isoform X3 | 1 | [symbol] | [symbol] | [symbol] | [symbol] | [symbol] | [symbol] | [symbol] | [symbol] | |
| peptidyl-prolyl cis-trans isomerase A isoform X2 | 1 | [symbol] | [symbol] | [symbol] | [symbol] | [symbol] | [symbol] | [symbol] | [symbol] | |
| peptidyl-prolyl cis-trans isomerase B | 1 | | [symbol] | [symbol] | | [symbol] | [symbol] | [symbol] | [symbol] | [symbol] |
| peptidyl-prolyl cis-trans isomerase C isoform X2 | 1 | [symbol] | [symbol] | [symbol] | [symbol] | [symbol] | [symbol] | [symbol] | [symbol] | [symbol] |
| peptidyl-prolyl cis-trans isomerase D isoform X2 | 1 | [symbol] | | | [symbol] | | | [symbol] | | |
| peptidyl-prolyl cis-trans isomerase FKBP10 isoform X1 | 1 | | [symbol] | [symbol] | | [symbol] | [symbol] | [symbol] | [symbol] | [symbol] |
| peptidyl-prolyl cis-trans isomerase FKBP1A isoform X2 | 1 | [symbol] | [symbol] | [symbol] | [symbol] | [symbol] | [symbol] | [symbol] | | [symbol] |
| peptidyl-prolyl cis-trans isomerase FKBP2 isoform X1 | 1 | | | [symbol] | | | [symbol] | [symbol] | | |
| peptidyl-prolyl cis-trans isomerase FKBP3 isoform X2 | 1 | [symbol] | [symbol] | [symbol] | [symbol] | [symbol] | [symbol] | [symbol] | [symbol] | [symbol] |
| peptidyl-prolyl cis-trans isomerase FKBP4 isoform X2 | 1 | [symbol] | [symbol] | [symbol] | [symbol] | [symbol] | [symbol] | [symbol] | [symbol] | [symbol] |
| peptidyl-prolyl cis-trans isomerase FKBP9 | 1 | [symbol] | [symbol] | [symbol] | [symbol] | [symbol] | [symbol] | [symbol] | [symbol] | [symbol] |
| phospholipase A-2-activating protein | 1 | [symbol] | | | [symbol] | | | [symbol] | | |
| phospholipase D3 | 1 | [symbol] | | | [symbol] | | | | | |
| procollagen-lysine,2-oxoglutarate 5-dioxygenase 1 isoform X3 | 2 | [symbol] | [symbol] | [symbol] | [symbol] | [symbol] | [symbol] | [symbol] | [symbol] | [symbol] |
| procollagen-lysine,2-oxoglutarate 5-dioxygenase 2 isoform X2 | 2 | [symbol] | | [symbol] | [symbol] | | [symbol] | | [symbol] | |
| protein disulfide-isomerase A3 precursor | 1 | | [symbol] | [symbol] | | [symbol] | [symbol] | | | |
| protein disulfide-isomerase A4 | 1 | [symbol] | [symbol] | [symbol] | [symbol] | [symbol] | [symbol] | | [symbol] | [symbol] |
| protein disulfide-isomerase A6 isoform X2 | 1 | [symbol] | [symbol] | [symbol] | [symbol] | [symbol] | [symbol] | | [symbol] | |
| protein disulfide-isomerase precursor | 1 | | [symbol] | [symbol] | | [symbol] | [symbol] | | [symbol] | [symbol] |
| protein S100-A10 | 2 | | [symbol] | [symbol] | | [symbol] | [symbol] | [symbol] | [symbol] | [symbol] |
| protein S100-A11 | 2 | [symbol] | [symbol] | [symbol] | [symbol] | [symbol] | [symbol] | [symbol] | [symbol] | [symbol] |
| protein S100-A13 | | [symbol] | | | [symbol] | | | | | |
| Protein S100-A13 | 2 | | [symbol] | [symbol] | | [symbol] | [symbol] | | | |
| protein S100-A4 | 2 | [symbol] | | [symbol] | [symbol] | | [symbol] | [symbol] | [symbol] | [symbol] |
| protein S100-A6 isoform X1 | 2 | [symbol] | [symbol] | [symbol] | [symbol] | [symbol] | [symbol] | [symbol] | [symbol] | [symbol] |
| putative phospholipase B-like 2 | 2 | | [symbol] | [symbol] | | [symbol] | [symbol] | [symbol] | [symbol] | [symbol] |
| pyruvate kinase PKM isoform X1 | 2 | [symbol] | | [symbol] | [symbol] | | [symbol] | [symbol] | | [symbol] |
| pyruvate kinase PKM isoform X2 | 2 | [symbol] | [symbol] | [symbol] | [symbol] | [symbol] | [symbol] | [symbol] | [symbol] | [symbol] |
| serine protease HTRA1 isoform X2 | 1 | | [symbol] | [symbol] | | [symbol] | [symbol] | [symbol] | [symbol] | [symbol] |
| serine protease HTRA2, mitochondrial | 1 | | | [symbol] | | | [symbol] | [symbol] | | |
| SPARC isoform X3 | 1 | | [symbol] | [symbol] | | [symbol] | [symbol] | [symbol] | | [symbol] |
| transforming growth factor beta receptor type 3 | 2 | [symbol] | [symbol] | | [symbol] | [symbol] | | [symbol] | [symbol] | |
| ubiquitin-60S ribosomal protein L40 isoform X1 | 1 | | | [symbol] | | | [symbol] | [symbol] | [symbol] | [symbol] |
| vimentin | 1 | [symbol] | [symbol] | [symbol] | [symbol] | [symbol] | [symbol] | [symbol] | [symbol] | [symbol] |

[symbol]

indicates data missing or illegible when filed

Prior work on LigaGuard™ technology introduced the paradigm of “flow-through affinity chromatography,” where an ensemble of discovered peptide ligands was immobilized on a chromatographic substrate to specifically capture HCPs, as well as other process- and product-related impurities from industrial CHO cell culture harvests. The first generation (G.1) LigaGuard™ resin comprised 9 peptides, and while capable of clearing a significant fraction of HCPs and ensure good mAb recovery (˜85%), could be perceived as inadequate in capturing all the species that the current literature has come to recognize as persistent, high-risk HCPs. This prompted the expansion of the list of peptide ligands by adding five sequences designed to target the escaping species. The resulting second generation (G.2) LigaGuard™ resin, presented in this present disclosure, provides a significant improvement in mAb recovery, with values consistently higher than 90%, and high monomer purity.

When challenged against different industrial CHO HCCFs that differed widely in terms of mAb titer, product properties and HCP titer and diversity, G.2 resin exhibited a remarkable robustness, demonstrating that the additional peptide ligands indeed strengthened the purification ability of the adsorbent. Furthermore, unlike the precursor G.1 resin whose HCP-binding activity decreased rather sharply with loading, G.2 resin achieved a consistently higher global HCP LRV (0.5-2) in comparison, across the entire load volume. The ability of G.2 resin to capture the bioprocess-persistent HCPs before they access the mAb purification pipeline, namely, the train of capture, intermediate purification, and final polishing, before they become a threat to product quality and patient's health is significant. Therefore, while applicable as a post-Protein A polishing step, LigaGuard™ adsorbents also seem well suited as a pre-Protein A adsorbent for frontal HCP removal, thus potentially (i) improving the performance and lifetime of expensive Protein A media and (ii) simplifying the optimization of intermediate purification and making further polishing steps optional if applicable. In this regard, LigaGuard™ adsorbents may ultimately lead to perfecting the mAb purification pipeline as a true platform, by substantially reducing the need of optimization (i.e., based on product, cell line, and upstream process conditions) and streamlining process development and validation. Due to its flow-through nature, utilizing the LigaGuard™ technology would also be very easy to integrate into continuous platforms for biomanufacturing that are currently being developed by various companies.

Furthermore, the LigaGuard™ technology is conductive to continuous, straight-through processes for manufacturing both mAb and non-mAb products. Morphing biomanufacturing towards straight-through processes, in fact, would offer major benefits including reducing the number and number of aqueous buffers, capital costs, and facilitate full process automation. Of growing interest is the development of Protein A-free mAb manufacturing and continuous production of viral vectors, where the LigaGuard™ technology could also play a key role.

### Example 4

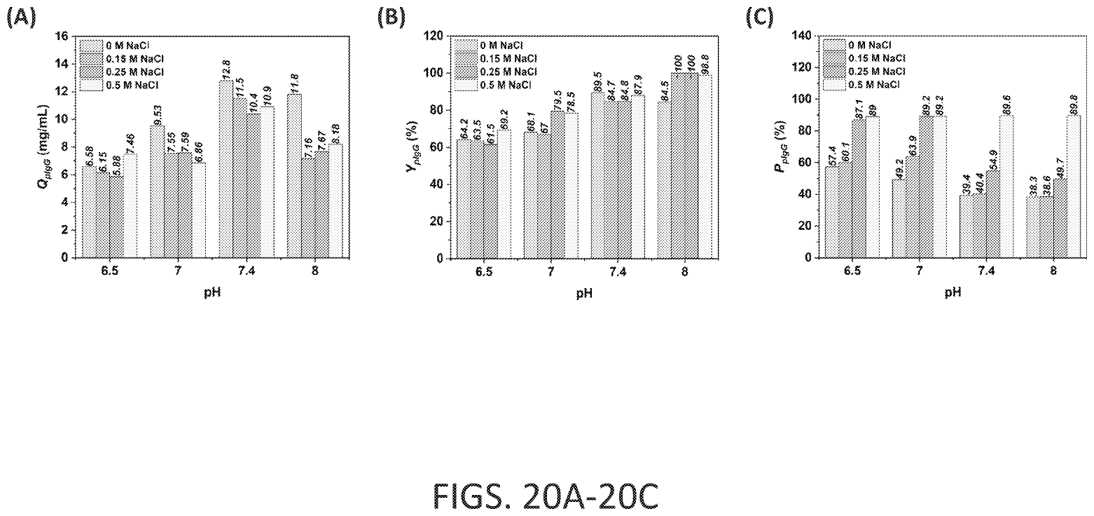

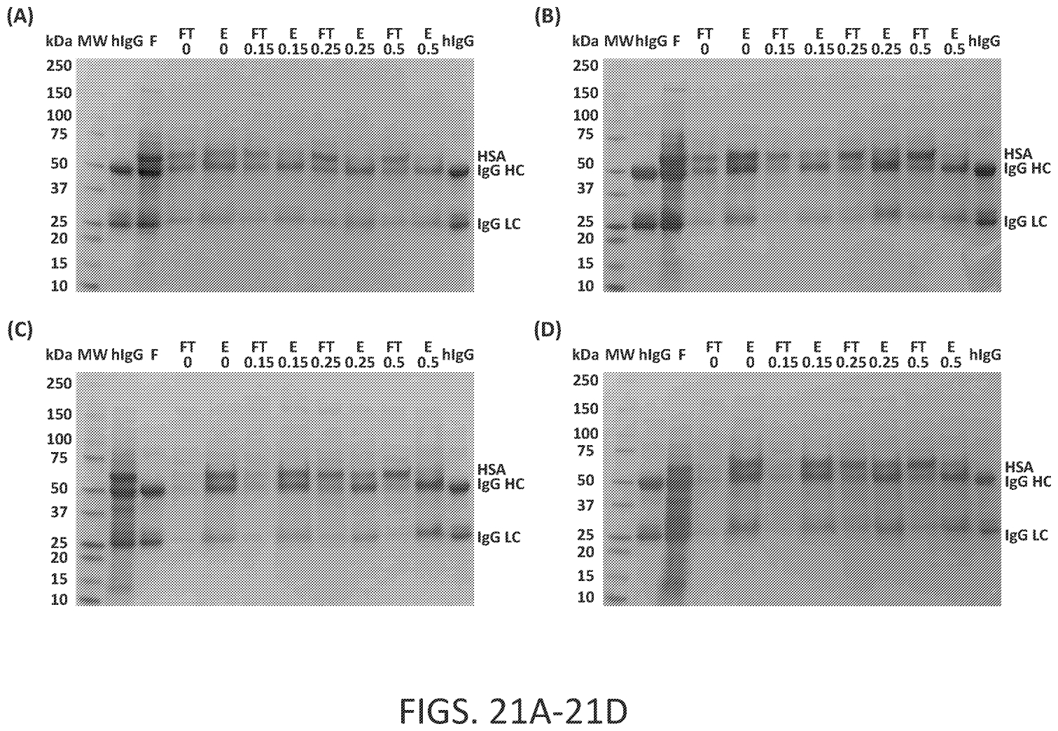

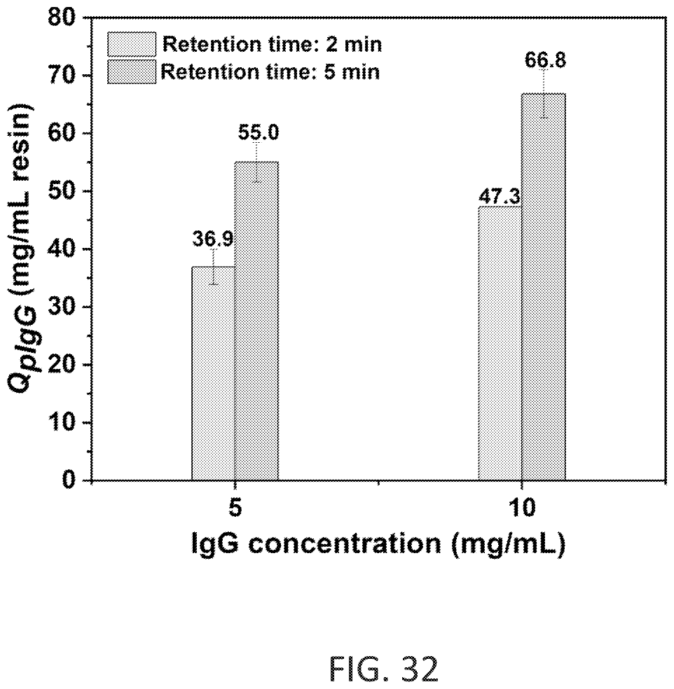

IgG purification from human Ig-rich paste using LigaTrap™ resin. LigaTrap™ resin has been developed for purifying γ-globulins from polyclonal and monoclonal sources. Compared to Protein A/G-based adsorbents, this adsorbent features comparable binding capacity and selectivity, improved lifetime, and a substantially lower cost, making it ideal for extracting pIgG from large volumes of pooled plasma. In this study, the performance of LigaTrap™ resin was initially evaluated against a human Ig-rich paste obtained via cold ethanol precipitation of plasma. To this end, the process conditions were optimized focusing on protein loading (mass of IgG loaded per volume of resin), residence time (FIG. 32), and composition and pH (6.5, 7.0, 7.4 and 8.0) of the binding and washing buffers. The eluted fractions were analyzed to determine pIgG adsorption as well as yield and purity (FIGS. 20 and 21).

As shown in FIG. 20, the pH of the binding buffer is the major determinant of pIgG binding capacity and selectivity on LigaTrap™ resin, whereas conductivity plays a rather minor role: at all values of NaCl concentration, in fact, pIgG binding and yield continuously increased as the binding pH ranged from 6.5 to 8.0, reaching their maximum of ˜11.4 mg/mL at pH 7.4 and ˜99.0% at pH 8.0, respectively; conversely, both binding and yield remained constant as the NaCl concentration increased, demonstrating that LigaTrap™ resin features salt-tolerant adsorption of pIgG.

These results are coherent with the composition of the LigaTrap ligand, which features a combination of hydrophobic, cationic, and hydrogen bonding moieties. As the isoelectric point of pIgG varies in the range 7.0-8.1, it stands to reason that binding is highest at 7.4, driven by hydrophobic and hydrogen bond interactions, which provide for salt-tolerant binding; additionally, as the pH is lowered to 4, elution was likely triggered by the electrostatic repulsion between the induced cations on pIgG and the ligands. When column loading is conducted at low salinity, the purity of eluted pIgG decreases with pH. This is imputed to anionic plasma proteins, such as α₁-antitrypsin (pI˜4.6), albumin (pI˜4.7), fibrinogen (pI˜5.5), and transferrin (pI˜6), that interact with the cationic moieties of the ligand. This, however, is effectively mitigated by increasing the conductivity of the binding and washing buffers, which afforded high pIgG capture, yield (≥85.0%), and purity (˜90.0%) (FIG. 21). Accordingly, the NaCl concentration of 0.5 M was adopted in both binding and washing buffer for the remainder of this study.

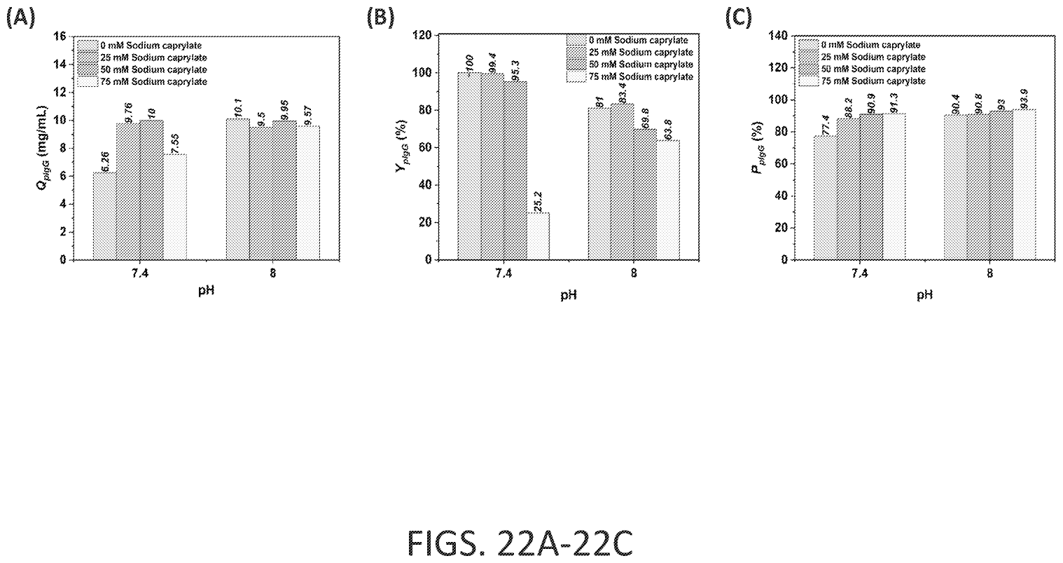

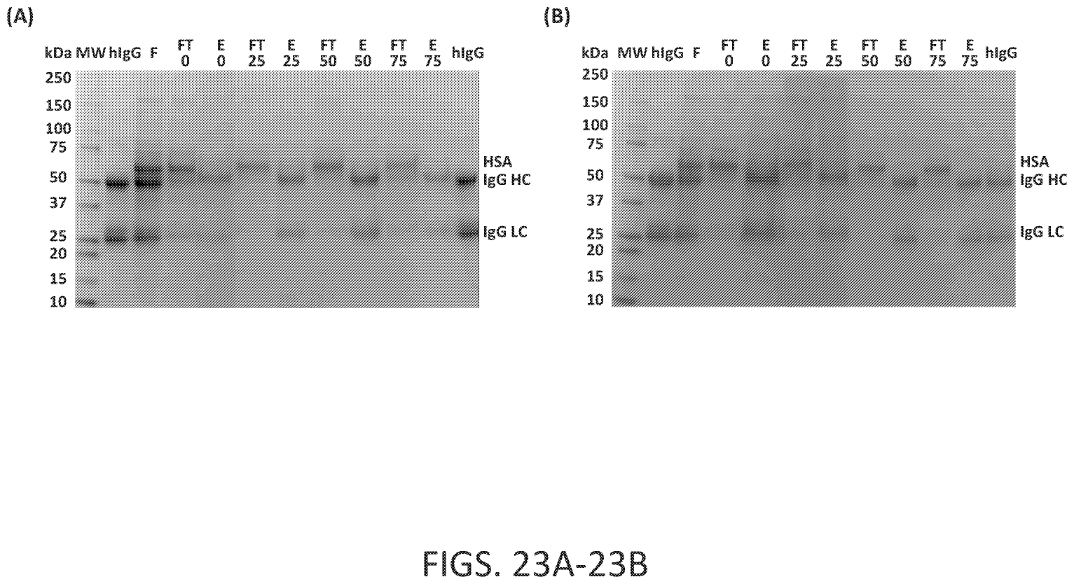

To further improve the performance of the LigaTrap™ resin, experiments were conducted to evaluate the addition of sodium caprylate to the binding and washing buffers to minimize the capture of highly abundant non-Ig serum proteins, chiefly albumin. The key chromatographic results—namely, pIgG binding, and elution yield (FIG. 22) and purity (FIG. 23)—suggest that the sodium caprylate substantially reduced the amount of bound non-Ig plasma proteins, bringing the purity of pIgG in the elution from ˜90% (no caprylate) to 94% (75 mM caprylate and pH 8.0); the rejection of albumin in particular is evident in the electrophoretic analysis of the flow-through fractions collected at pH 8.0 (FIG. 23). The addition of caprylate, however, lowers pIgG yield from ˜99% (no caprylate) to 64% (75 mM caprylate and pH 8.0). In this regard, when present at higher concentration, it is likely that the caprylate anions progressively saturate the binding sites on albumin and other plasma proteins, eventually adsorbing on IgG and increasing the effective hydrophobicity of its surface; the same trend has been observed in prior work using the mixed-mode resin MEP HyperCel. Overall, column loading at pH 7.4 afforded values of yield that are significantly and consistently higher (˜95-100%) than those obtained by loading at pH 8.0 (˜63-83%). Considering the high yield (˜95%) and purity (˜91%), 0.1 M phosphate buffer at pH 7.4 added with 0.5 M NaCl and 25 mM sodium caprylate was selected as binding and washing buffer for the remainder of this study.

### Example 5

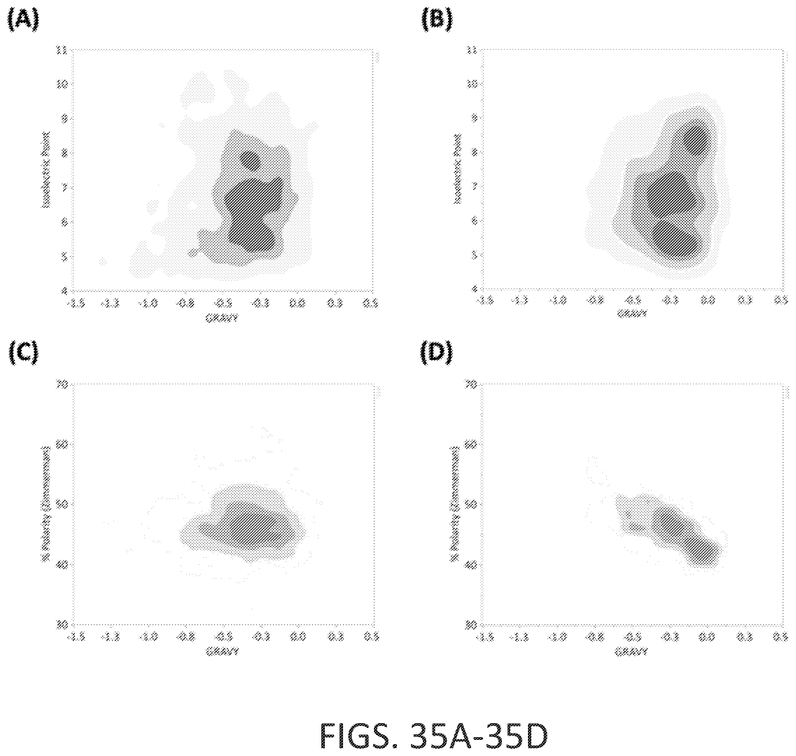

Purification of pIgG from cryo-rich and cryo-poor plasma in flow-through mode using LigaGuard™ adsorbents. The LigaGuard™ adsorbent has been originally developed for purifying monoclonal antibodies from Chinese Hamster Ovary (CHO) cell culture supernatants in flow-through mode. The resin is functionalized with an ensemble of peptide ligands that capture a broad spectrum of protein impurities differing by composition, post-translational modification, size, and titer, while allowing the antibody product to flow through unbound. To this end, the LigaGuard™ peptides operate as advanced mixed-mode ligands, wherein each peptide targets multiple proteins through a combination of electrostatic and hydrophobic interactions, and hydrogen bonds. Notably, the proteins secreted by CHO cells and human plasma proteins show a remarkable biocheminformatic similarity, as illustrated by the comparison of the respective values of sequence-based isoelectric point (pI), polarity (Zimmerman scale), and GRand AVerage of hYdropathicity (GRAVY) index (FIG. 35). This homology prompted adoption of the LigaGuard™ resin for purifying IVIg from plasma in the same manner utilized for purifying monoclonal antibodies from CHO cell culture harvests.

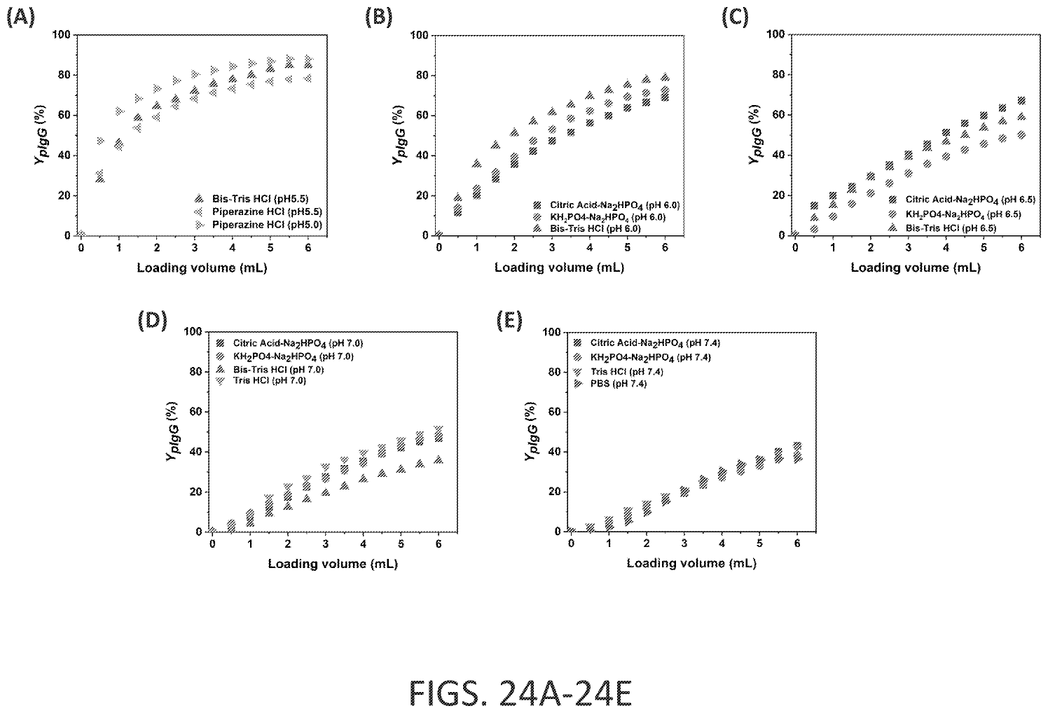

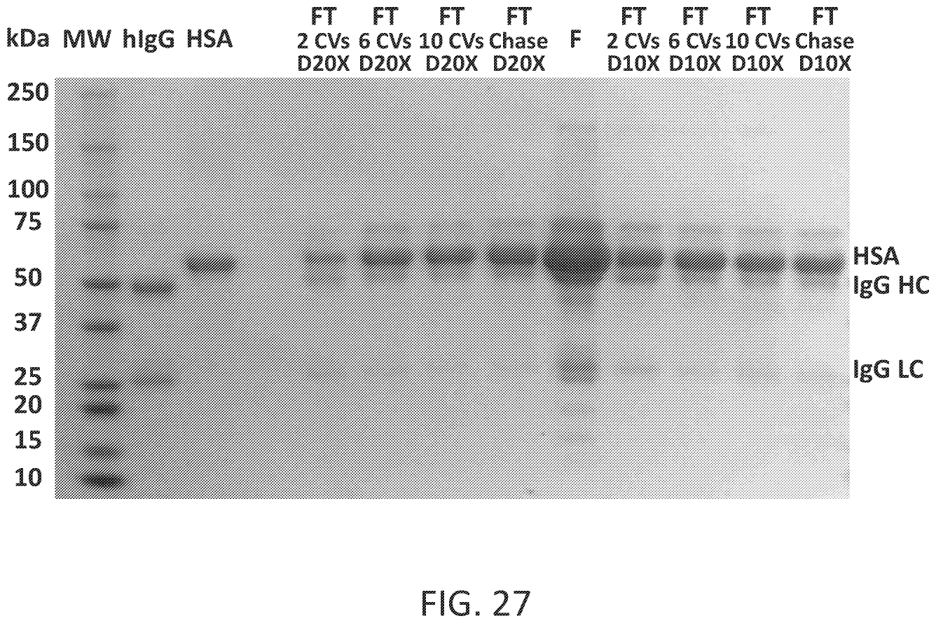

At the same time, the higher complexity of plasma, where the ratio of Ig vs. non-Ig proteins is about 2·10⁵ ppm, compared to that of recombinant sources, where the same ratio varies between 1-2·10⁵ ppm, poses the need to optimize both the composition of the LigaGuard™ ligands and the chromatographic protocol. First, the chromatographic process was optimized by evaluating the effect of composition and pH of the running buffer on the recovery of pIgG and the retention of non-Ig plasma proteins. Injections of either pure human pIgG (˜3.0 mg/mL) or Ig-depleted plasma (˜5.0 mg/mL) were performed to select a value of load and a binding condition that enable flow-through purification of pIgG from plasma. The resultant profiles of pIgG yield (Y_{pIgG}) vs. loading volume are collated in FIG. 24; the corresponding values of breakthrough ratio, namely the ratio of pIgG titer in the effluent vs. feedstock (C_{pIgG}/C_{pIgG}*), and binding (Q_{pIgG}) are reported in FIG. 35A and FIG. 35B, respectively. The profiles of non-Ig plasma proteins capture (Q_{PP}) vs. loading volume are collated in FIG. 27, while the corresponding values of breakthrough ratio (C_{PP}/C_{PP}*) are reported in FIG. 35C. By highlighting the role of buffer composition and pH on the binding of plasma proteins, these results corroborate the classification of the LigaGuard™ peptides as advanced multi-modal ligands. First, decreasing pH improved binding selectivity (Y_{pIgG} and Q_{PP}): at the loading volume of 5 mL—corresponding to 10 column volumes (CVs), at which the LigaGuard™ adsorbent is saturated with non-Ig plasma proteins-decreasing the loading pH from 7.4 to 5.5 improved Y_{pIgG} from ˜35.0% to ˜80.0%; as the environment becomes more acidic, in fact, both the ligands (pI=7.8-12.5) and pIgG (pI=6.1-8.5) acquire a net positive charge, and undesired pIgG capture is prevented by electrostatic repulsion.

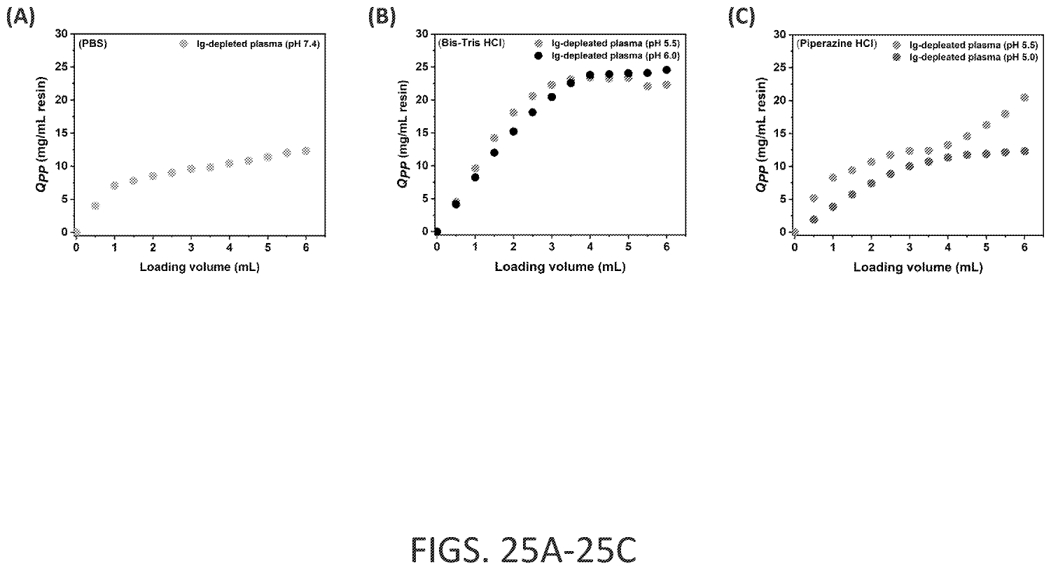

Compared to pH, buffer composition has a minor, yet still noticeable effect on pIgG yield. At pH 7.4, in fact, the values of Y_{pIgG} obtained with different binding buffers are virtually indistinguishable; at pH 6.5-7.0, a 10% difference in Y_{pIgG} across the entire range of loading volume is observed between the monovalent (Bis-Tris HCl) and the trivalent (citric acid-Na₂HPO₄) buffer; as the pH decreases further to 5.0-6.0, the difference in Y_{pIgG} among the various buffers grows to 20%, with piperazine HCl buffer at pH 5.0 affording the highest product yield, namely 87% at the cut-off loading volume of 10 CVs. The capture of pIgG during the initial stages of binding—namely, up to a loading of 3 CVs—is due to the makeup of the load: in the absence of non-Ig proteins competing with pIgG for the peptide ligands, a small product loss due to non-specific adsorption is inevitable. In this context, Y_{pIgG} can be increased by “chasing” the loading with buffer C, which increased Y_{pIgG} from 75-77% to 87%. While operating binding under acidic conditions reduced the loss of pIgG, experiments were not conducted to explore any buffer with pH<5.0 to avoid the flow-through of albumin (pI˜4.7) and the risk of pIgG denaturation and aggregation. Accordingly, 20 mM Bis-Tris HCl buffer was adopted at pH 6.0 and 5.5 and 20 mM piperazine HCl buffer was adopted at pH 5.5 as buffers for pIgG purification from human plasma in flow-through mode. Next, the capture of non-Ig plasma proteins by the LigaGuard™ adsorbent was evaluated using comprising of filtered and Ig-depleted plasma as feedstock (FIG. 25).

When operating at pH 7.4, substantial flow-through of non-Ig plasma proteins was observed: upon reaching a loading a volume of ˜1-1.5 mL (2-3 CVs), the C_{PP}/C_{PP}* rapidly rose to ˜0.8, thus disqualifying PBS as a mobile phase for LigaGuard™; to some surprise, 20 mM piperazine-HCl buffers at pH 5.0 and 5.5 failed to afford appreciable capture of plasma proteins and were also excluded. Conversely, loading in Bis-Tris buffer led to a notable increase in the capture of non-Ig plasma proteins: at the loading volume of 1 mL (2 CVs), the Y_{pIgG} reached 50% while only 4% of the loaded non-Ig plasma proteins are found in the effluent, corresponding to a Q_{PP} of 8.9 mg/mL; at the cut-off value of loading (10 CVs), the Y_{pIgG} reached 80% and approximately 53.1% of the non-Ig plasma proteins has been captured, corresponding to a Q_{PP} of ˜23.7 mg/mL.

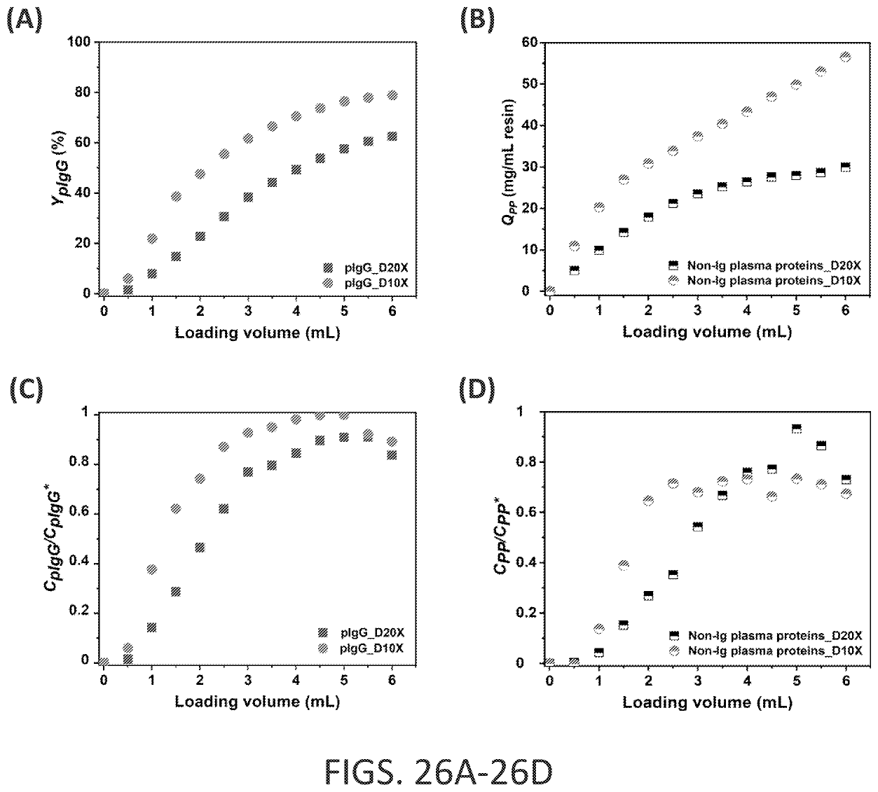

Capitalizing on these results, experiments were conducted to evaluate the LigaGuard™ adsorbent for the flow-through purification of pIgG from cryo-rich plasma (total protein titer of ˜70 mg/mL, Ig titer of 9.7 mg/mL) [47]; the plasma was diluted by either 10- or 20-fold with binding buffer to achieve the ionic strength and pH needed for effective capture of non-Ig proteins by LigaGuard™ peptides. The resultant profiles of Y_{pIgG} and Q_{PP} vs. loading volume are collated in FIG. 26A and FIG. 26B; the corresponding profiles of C_{pIgG}/C_{pIgG}* and C_{PP}/C_{PP}* are reported in FIG. 26C and FIG. 26D.

Most notably, Q_{pIgG} was not affected by the dilution factor and plateaued at ˜2.5 mg per mL of resin. Conversely, upon decreasing feedstock dilution from 20-fold to 10-fold, the Q_{PP} at cut-off loading doubled from 25 mg/mL to 50 mg/mL; this translated in a global Y_{pIgG} of ˜76.4% and a 1.7-fold reduction of non-Ig plasma proteins from 10-fold diluted plasma, corresponding to a 0.59-fold product enrichment in the effluent (Table 4). The improvement in product yield at the cut-off loading with plasma in lieu of pure pIgG solution (Y_{pIgG}˜70%) likely resulted from the presence of non-Ig plasma proteins outcompeting pIgG binding; furthermore, the adsorbed pIgG was stripped from the column by chasing the loading with 2 CVs binding buffer, which increased Y_{pIgG} up to 78.8% without compromising the purity of the pooled effluent (FIG. 27 and Table 4).

TABLE 4
Values of Y_{pIgG}, reduction of non-Ig plasma proteins, and
pIgG enrichment factor in the effluent compared to feedstock
obtained by loading 5 mL (10 CVs) of diluted plasma onto
LigaGuard ™ adsorbent at 1 min residence time.

| Diluted | | | Reduction of non-Ig | | pIgG enrichment | |
|---|---|---|---|---|---|---|
| plasma | Y_{pIgG} | | plasma proteins | | factor | |
| feed- | Loading | Loading + | Loading | Loading + | Loading | Loading + |
| stock | only | chasing | only | chasing | only | chasing |
| 10-fold | 77% | 80% | 4.7 | 4.9 | 1.3 | 1.0 |
| 20-fold | 58% | 63% | 5.9 | 5.0 | 1.0 | 1.2 |

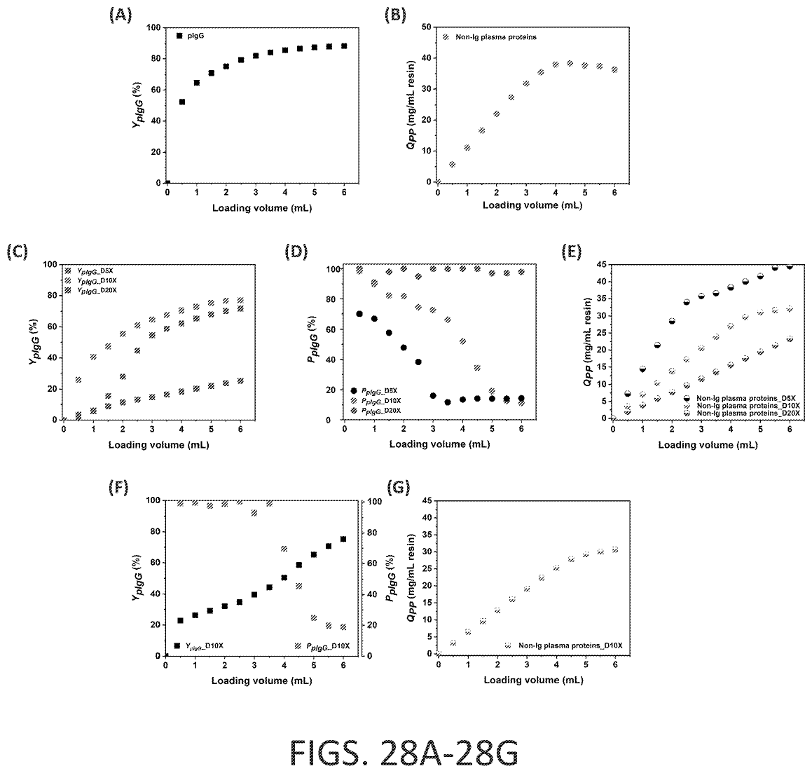

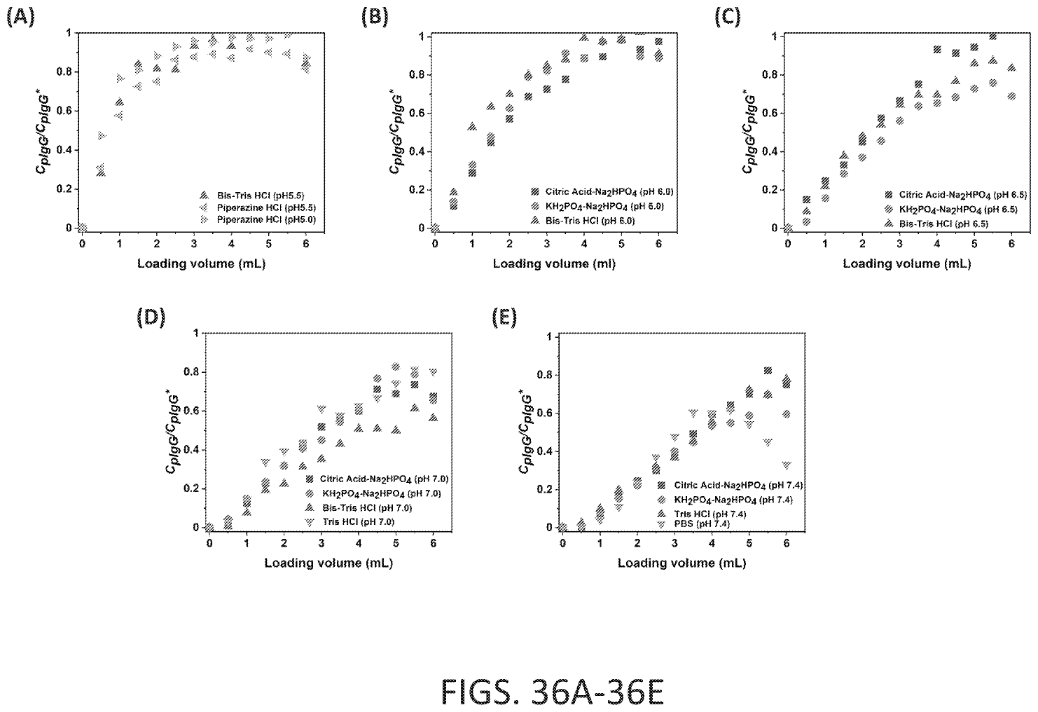

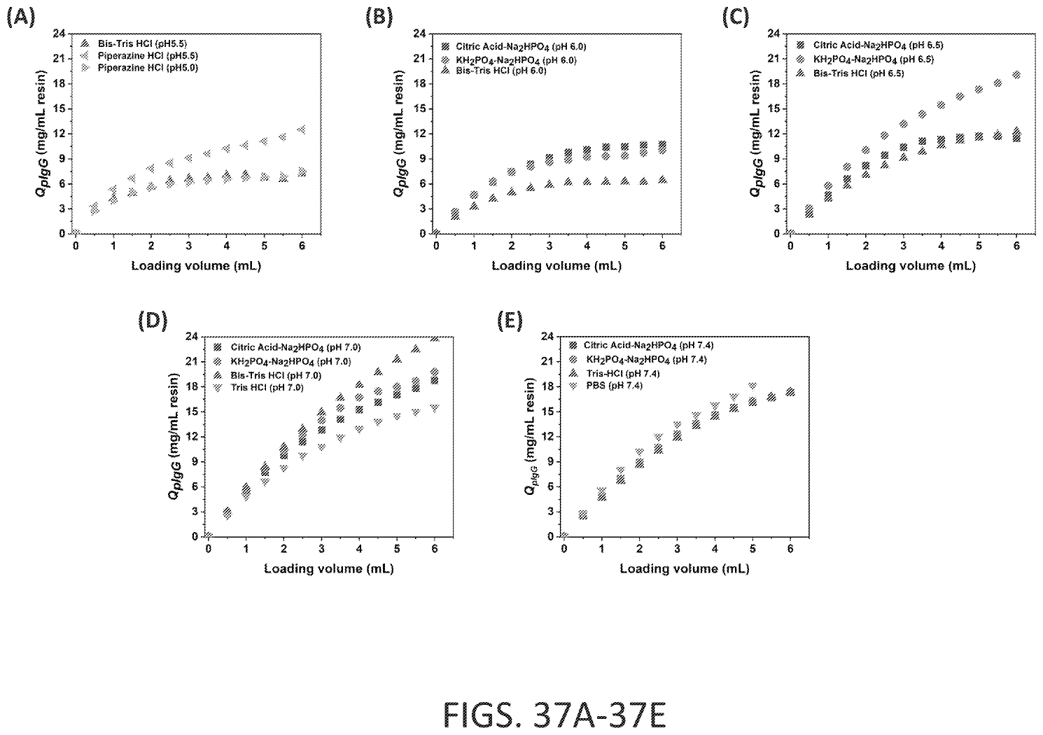

To increase both Y_{pIgG} and pIgG enrichment factor in the effluent, the LigaGuard™ resin was modified by improving its multi-modal binding character: specifically, the anion exchange component was strengthened by quaternizing the nitrogen groups displayed on the cationic residues, and additional binding modalities were introduced by integrating polar and thiophilic moieties. It was anticipated that the combination of amine quaternization and pH 6.0 would increase the capture of non-Ig plasma proteins, which are for the most part anionic, while reducing the capture of pIgG. The resultant profiles of Y_{pIgG} and Q_{PP} vs. loading volume collated in FIG. 28, and the corresponding values of C_{pIgG}/C_{pIgG}* and Q_{pIgG}, and C_{PP}/C_{PP}* and Q_{PP} reported in FIG. 36 and FIG. 37, demonstrate a remarkable improvement in both product yield and capture of non-Ig plasma proteins by this second-generation LigaGuard™ adsorbent.

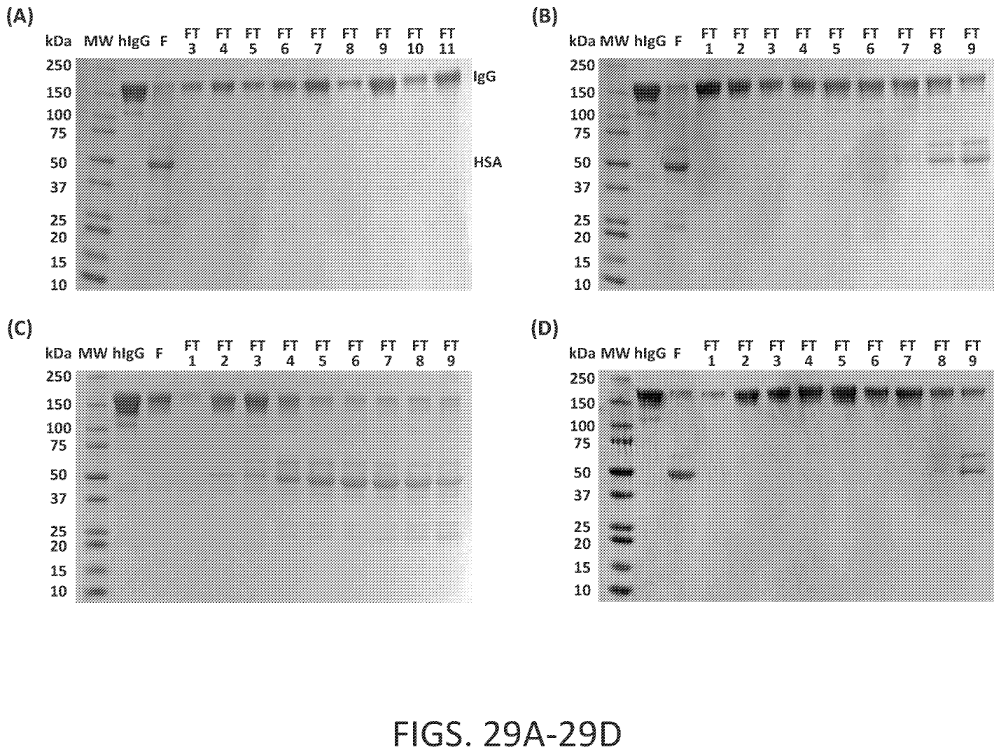

Following on these results, the flow-through purification of pIgG from diluted cryo-rich plasma was iterated. The resulting profiles of Y_{pIgG} and Q_{PP} vs. loading volume are reported in FIG. 28C and FIG. 28E; the corresponding profiles of C_{pIgG}/C_{pIgG}* are reported in FIG. 37. The loading of 20-fold diluted plasma (pIgG˜0.6 mg/mL; non-Ig plasma proteins ˜3.9 mg/mL) resulted in efficient flow-through purification: at the cut-off loading (10 CVs), the cumulative pIgG purity in the effluent reached 98.1% (FIG. 29A); the global yield, on the other hand, reached 25.4% only: the low protein concentration in the feedstock is unlikely to match the high binding capacity of the second-generation LigaGuard™ and prevent undesired capture of IgG via weak partitioning mechanism, as described in prior work. Accordingly, the loading of 10-fold diluted plasma resulted in a significant increase in pIgG yield with high purity; specifically, at the loading volume of 4.0 mL (8 CVs), Y_{pIgG} and Q_{PP} respectively reached ˜71% and 27 mg per mL resin, corresponding to a cumulative product purity of ˜80%; beyond this point, however, a significant amount of non-Ig proteins flow through the column, lowering product purity to ˜70% at the loading volume of 5.5 mL (FIG. 29B). Finally, the loading of 5-fold diluted plasma showed a significant loss of product yield and purity: upon loading 5 CVs, in fact, non-Ig plasma proteins began breaking through (C_{PP}/C_{PP}*˜5%) while the Y_{pIgG} was still relatively low (˜61%); yield increased appreciably through the subsequent loading phase, although product purity dropped significantly, reaching 76% at 10 CVs (FIG. 28E). Collectively, these results indicate that the 10-fold dilution ratio is ideal for stripping non-Ig plasma proteins using the LigaGuard™ adsorbent prior to, or in lieu of, affinity-based capture to improve the performance of subsequent chromatographic steps and maximize product yield and purity.

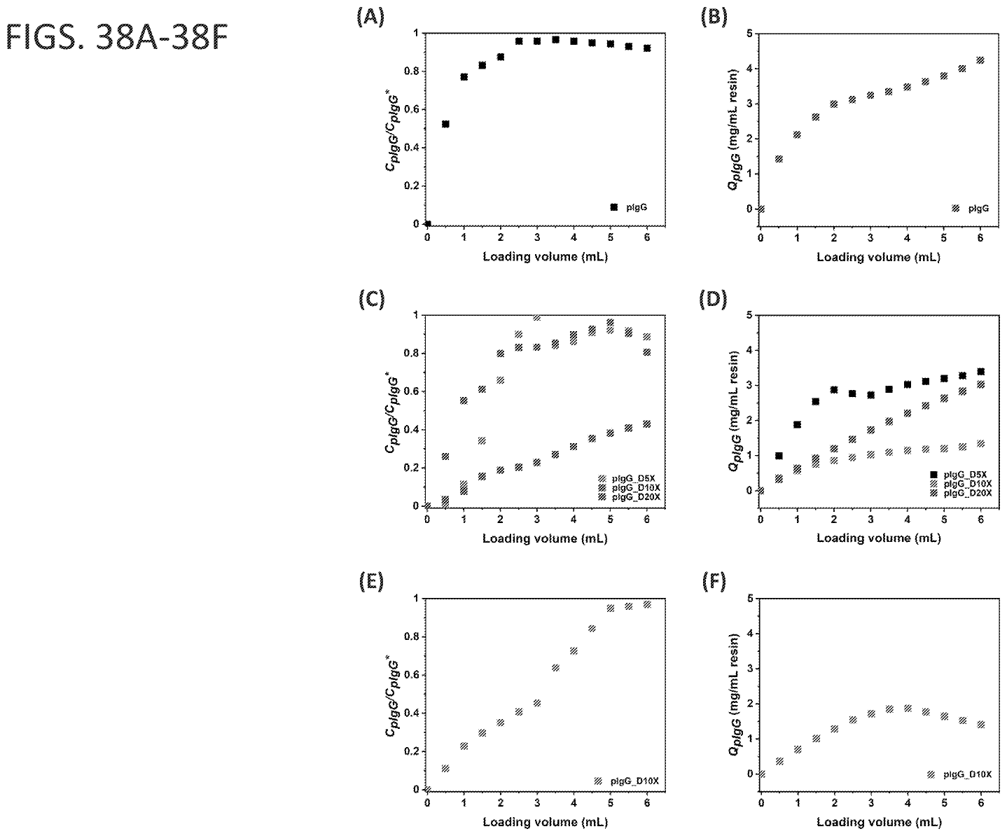

The optimal loading conditions were implemented to purify pIgG from cryo-poor plasma. The resulting profiles of product yield and purity vs. loading volume reported in FIG. 28F and FIG. 28G (the profiles of C_{pIgG}/C_{pIgG}* are in FIG. 38) indicate a purification performance similar to that obtained with cryo-rich plasma, with Y_{pIgG}˜50% and P_{pIgG}>93% (Q_{PP}>25.4 mg per mL of resin) obtained at the loading volume of 8 CVs. Interestingly, the Y_{pIgG} profile features two slopes, namely ˜7%/CV and ˜14%/CV, respectively before and after loading 7 CVs; this value also demarcates between the collection of high-purity effluent and the breakthrough of non-Ig plasma proteins, which lower the cumulative P_{pIgG} from 99% to 82% at 10 CVs (FIG. 29D). The profile of Q_{PP}, in fact, shows an inflection above 10 CVs, indicating the saturation of the LigaGuard™ adsorbent at 30 mg/mL, coherently with prior measurements.

### Example 6

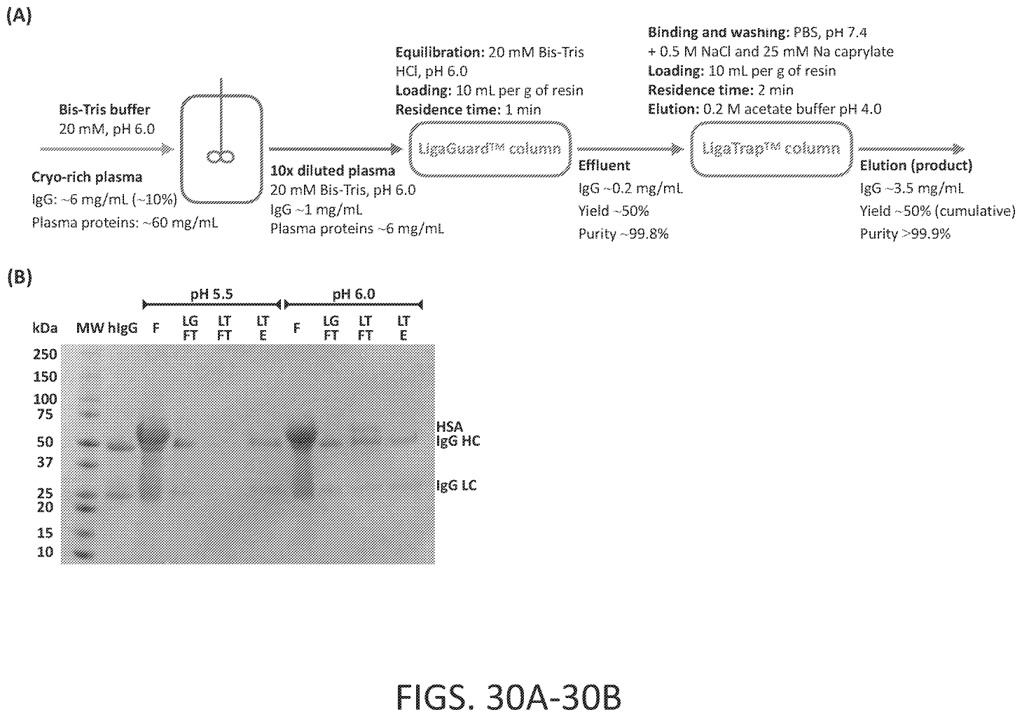

Purification of pIgG from cryo-rich plasma using a two-step process: Guard-Capture vs. Capture-Polish. Prior work demonstrated the use of LigaGuard™ resin as a scrubber of process-related impurities prior to the affinity capture step in a process purification for monoclonal antibodies. Analogous to that study, the purification of pIgG from plasma was attempted using a two-column process comprising a LigaGuard™ adsorbent that captures the non-Ig plasma proteins in flow-through followed by a LigaTrap™ adsorbent that operates in bind-and-elute mode to isolate and concentrate pIgG. The loading of diluted cryo-rich plasma was “chased” with buffer C, and the pooled effluent was adjusted to pH 7.4 and loaded onto LigaTrap™ adsorbent. The “guard-capture” process conditions detailing loading, buffer composition, and residence time, along with the resulting values of Y_{pIgG} and P_{pIgG} in the fractions across the two-column process are listed in FIG. 30A, while the electrophoretic analysis is shown in FIG. 30B. Notably, the depletion of non-Ig plasma protein by the LigaGuard™ adsorbent enabled a remarkable increase in the purity of pIgG eluted from the LigaTrap™ adsorbent (P_{pIgG}˜99.9%). Most importantly, the capture of albumin, the major contaminant in the affinity capture step, greatly facilitated the clearance of the residual impurities by LigaTrap™ adsorbent, thus improving its effective binding capacity and selectivity. This process design, however, suffers from limited throughput. The high concentration of non-Ig plasma protein in the feed (6.0 mg/mL) and the binding capacity of the LigaGuard™ adsorbent (32 mg per mL of resin), in fact, pose a limit to the volume of feedstock that can be fed into the two-column process.

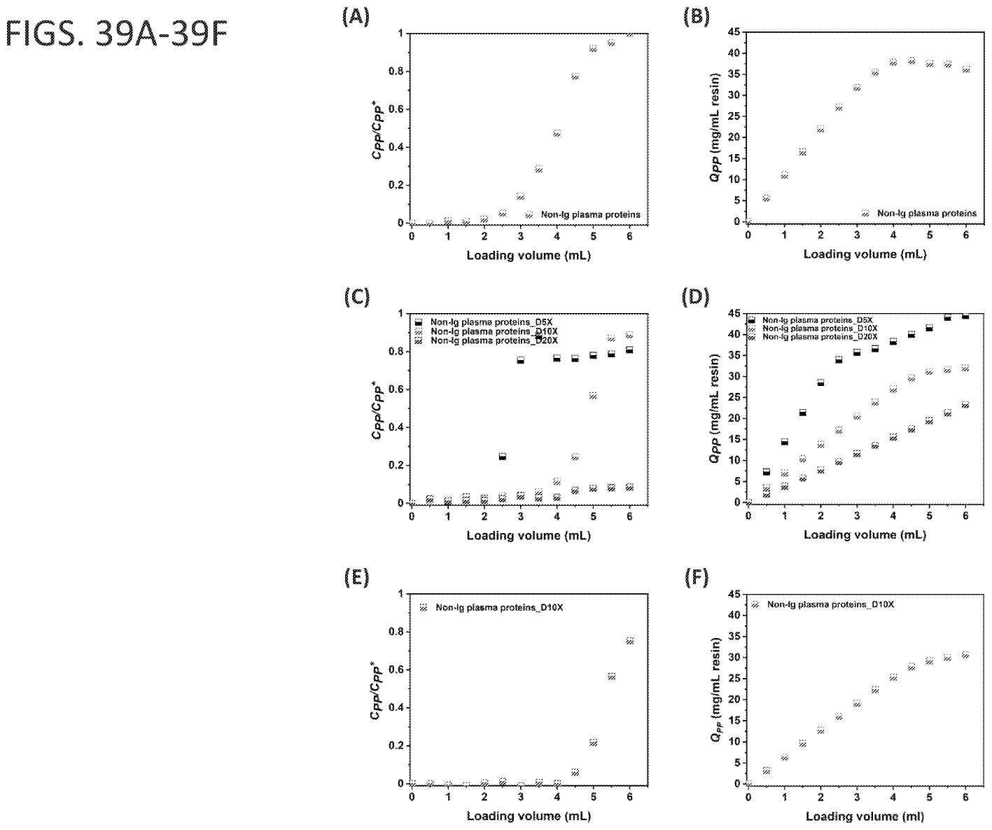

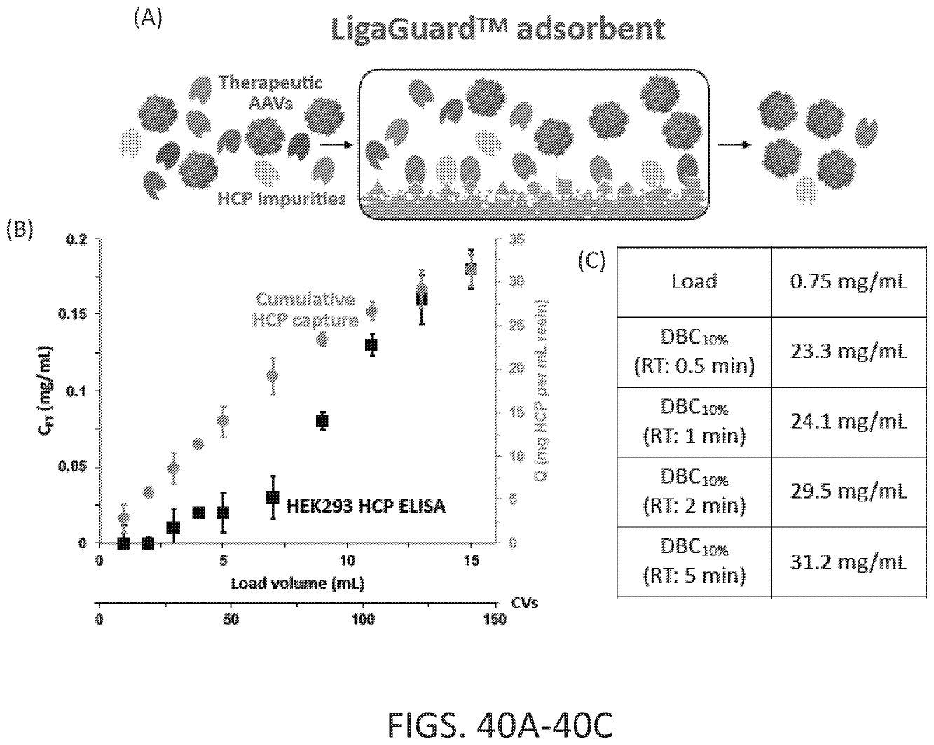

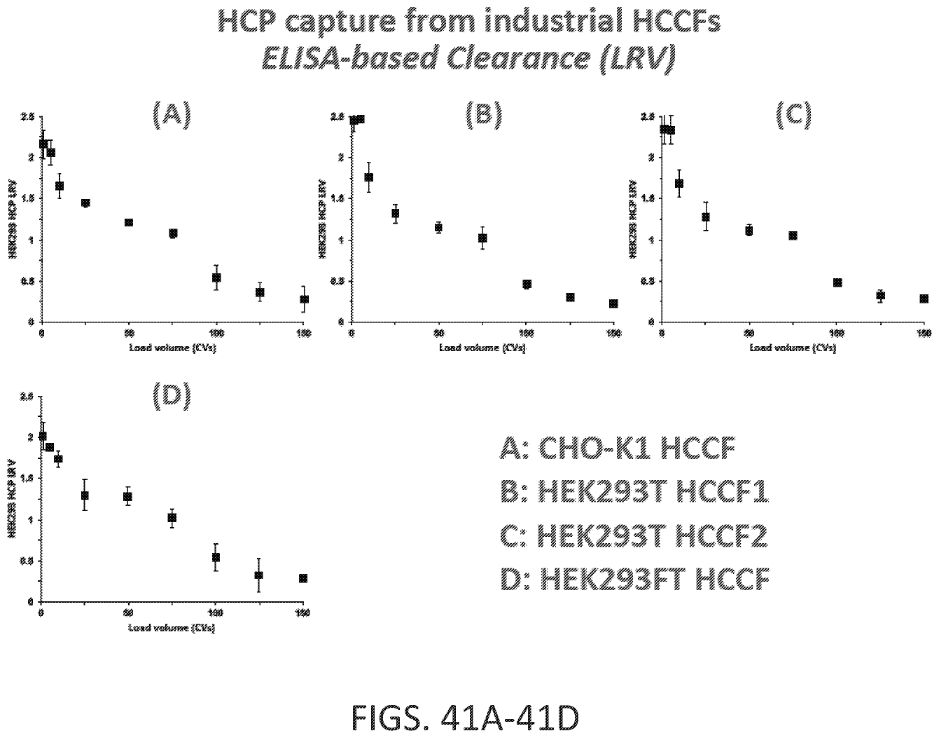

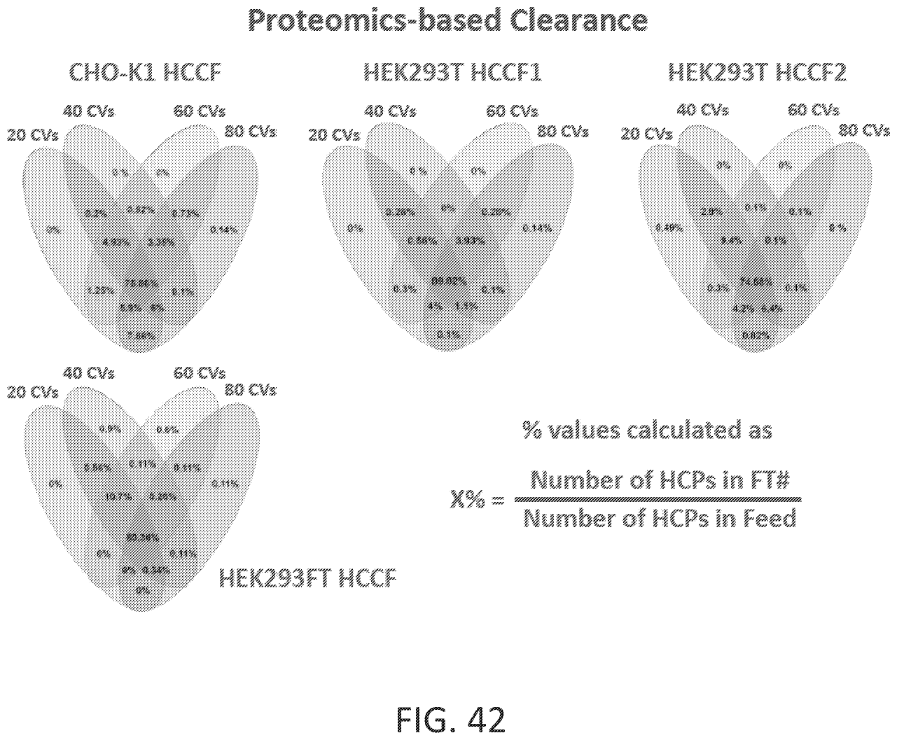

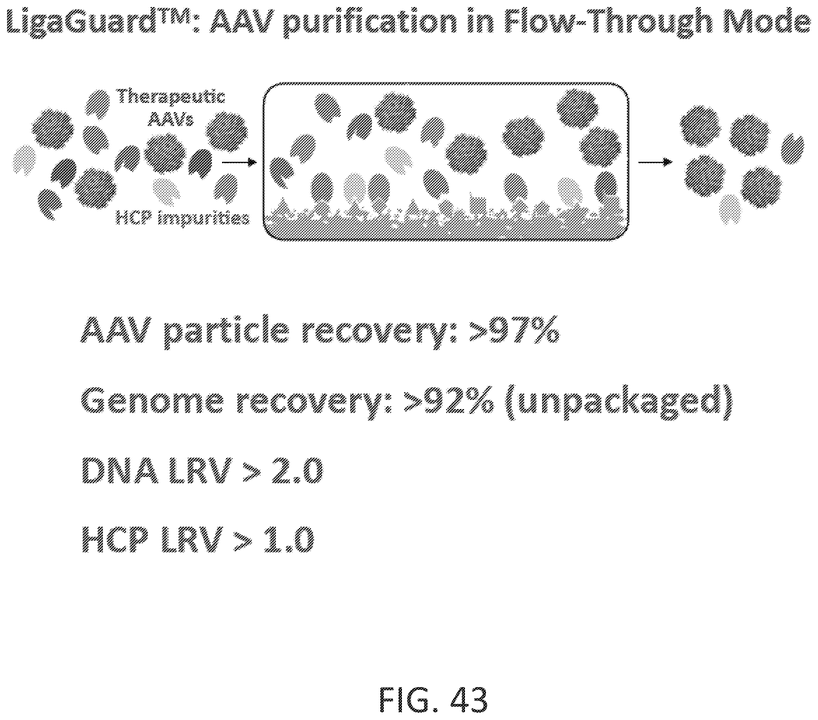

The global yield of the two-column process is evidently limited by the first step (Y_{pIgG}˜49.9%). To address this challenge, the pH of feedstock was lowered from 6.0 to 5.5 to enhance the electrostatic repulsion between IgG and the second-generation LigaGuard™ adsorbent (FIG. 39). This process adjustment raised the global yield to 63.8%, without altering final product purity (99.7%); for reference, the values of global pIgG yield obtained by processing Ig-rich Cohn fractions via ion exchange chromatography range between 40-70%.

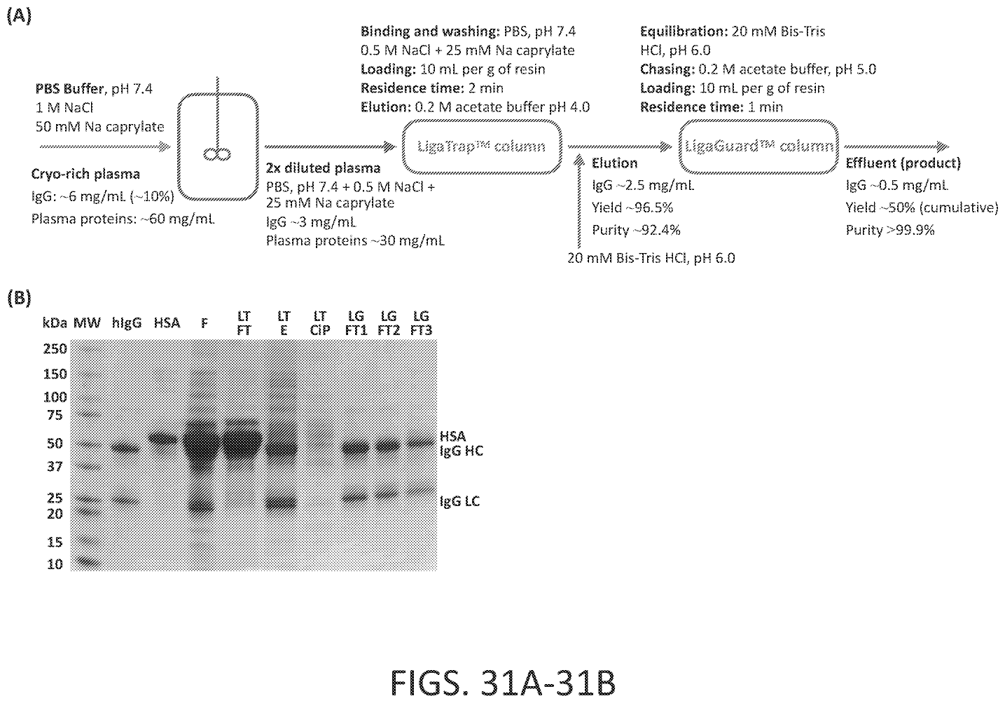

In the attempt to further pIgG recovery and productivity, an alternative two-column process was proposed comprising an affinity-based capture step using LigaTrap™ adsorbent in bind-and-elute mode followed by a polishing step using the LigaGuard™ adsorbent operating in flow-through mode. The “capture-polish” process diagram, conditions, the resulting values of Y_{pIgG} and P_{pIgG}, and electrophoretic analysis of the process fractions are reported in FIG. 31.

Notably, preponing product capture improved substantially the global recovery (Y_{pIgG}˜82.3%), while polishing still secured a high final product purity (P_{pIgG}˜98.8%). Furthermore, the binding capacity of the LigaTrap™ adsorbent enabled a 1.7-fold increase in the volume of plasma processed by the “capture-polish” compared to that enabled by the “guard-capture” using identical column volumes. On the other hand, the “capture-polish” imposes an intermediate step of buffer adjustment of the elution stream from the LigaTrap™ adsorbent prior to loading into the LigaGuard™ adsorbent, which lengthens the process and makes it less streamlined; furthermore, conducting the polishing in flow-through mode reduces the product concentration, thereby imposing a subsequent ultrafiltration step.

The performance of the two alternative process designs is summarized in Table 5, which reports, together with product yield and purity, the corresponding values of enrichment of pIgG and the clearance of non-Ig plasma proteins in the product stream. The remarkable levels of purification achieved with the first process configuration demonstrate the potential of the proposed technology for the purification of plasma-derived therapeutics.

TABLE 5
Values of Y_{pIgG}, P_{pIgG}, reduction of non-Ig plasma proteins, and
pIgG enrichment factor in the effluent compared to feedstock obtained
by loading 1 mL cryo-rich plasma onto LigaGuard ™ and
LigaTrap ™ adsorbents at different resin sequence and loading pH.

| | | | | | Reduction of | | | |
|---|---|---|---|---|---|---|---|---|
| | | | | | non-Ig | | pIgG enrichment | |
| Two- | Y_{pIgG} | | P_{pIgG} | | plasma proteins | | factor | |
| column | First | | First | | First | | First | |
| process | column | Global | column | Global | column | Global | column | Global |
| LG + LT | 66.6% | 63.8% | 97.2% | 99.7% | 22 | 241 | 76 | 870 |
| (pH 5.5) | | | | | | | | |
| LG + LT | ~50% | ~50% | 99.8% | >99.9% | 37 | 366 | 134 | 1362 |
| (pH 6.0) | | | | | | | | |
| LT + LG | 96.5% | 82.3% | 92.4% | 98.8% | 15 | 59 | 56 | 230 |
| (pH 6.0) | | | | | | | | |

Taken together, these results demonstrate the chromatographic purification of immunoglobulin G (IgG) from human plasma using a two-column process integrating the peptide-based adsorbents LigaGuard™, which captures non-Ig plasma proteins in flow-through mode, and LigaTrap™, which isolates IgG in bind-and-elute. Buffer composition and column loading were optimized for both adsorbents. Two process configurations were evaluated. In the first design, plasma was fed to a LigaGuard™ column to capture plasma proteins, the effluent was loaded on the LigaTrap™ column, and the bound IgG was eluted with 63.8% global recovery and 99.7% purity; in comparison, Protein G agarose afforded ˜67% recovery and 97.2% purity. In the alternative design, the LigaGuard™ column was utilized to polish the LigaTrap™ elution stream, affording 82.3% global recovery and 98.8% purity. Collectively, these results demonstrate the potential of a fully chromatographic process for purifying polyclonal IgG from plasma feedstocks.

### Example 7

Dynamic pIgG binding capacity of LigaTrap™ resin. The dynamic binding capacity at 10% breakthrough (DBC_{10%}, mg/mL resin) of human polyclonal IgG (pIgG) on LigaTrap™ resin was measured in non-competitive conditions (i.e., pure IgG in PBS, pH 7.4) at two values of pIgG titer, namely 5 and 10 mg/mL, and two values of residence time, namely 2 and 5 min. Notably, increasing the residence time from 2 min to 5 min, the DBC_{10%} increased dramatically from 49.0% to 55.0 mg/mL at the pIgG concentration of 5 mg/mL, and from 41.1% to 66.8 mg/mL at 10.0 mg/mL (FIG. 32). This can be ascribed to the relatively small pore diameter (<100 nm) of the crosslinked agarose resin utilized to build the LigaTrap™ adsorbent. Given the high affinity of the peptoid ligand for human IgG (K_{D}˜10⁻⁷ M), it was concluded that the binding capacity of LigaTrap™ resin is kinetically controlled, i.e., it is determined by the mass transfer of pIgG from the liquid phase to the ligand displayed on the pore's surface of the resin beads. Based on these results, the loading time of 5 min was adopted for all the IgG purification studies in bind-and-elute mode using LigaTrap™ resin in this work.

### Example 8

Purification of pIgG from human cryo-rich plasma using LigaTrap™ resin. Large-scale manufacturing of IVIg is dominated by the Cohn-Oncley process or its derivations, such as the Kistler-Nitschmann process, which employ cold ethanol and caprylate to precipitate plasma into fractions enriched with different segments of the blood protein population. Recently, however, chromatography technology has appeared in the landscape of IVIg purification: ion-exchange, hydrophobic charge induction, and peptide-based affinity resins have in fact been demonstrated for the purification of pIgG from Cryoprecipitate (a concentrate of high molecular-weight plasma proteins obtained by precipitating frozen plasma via slowly thawing at 1-6° C.) or Ig-rich fractions.

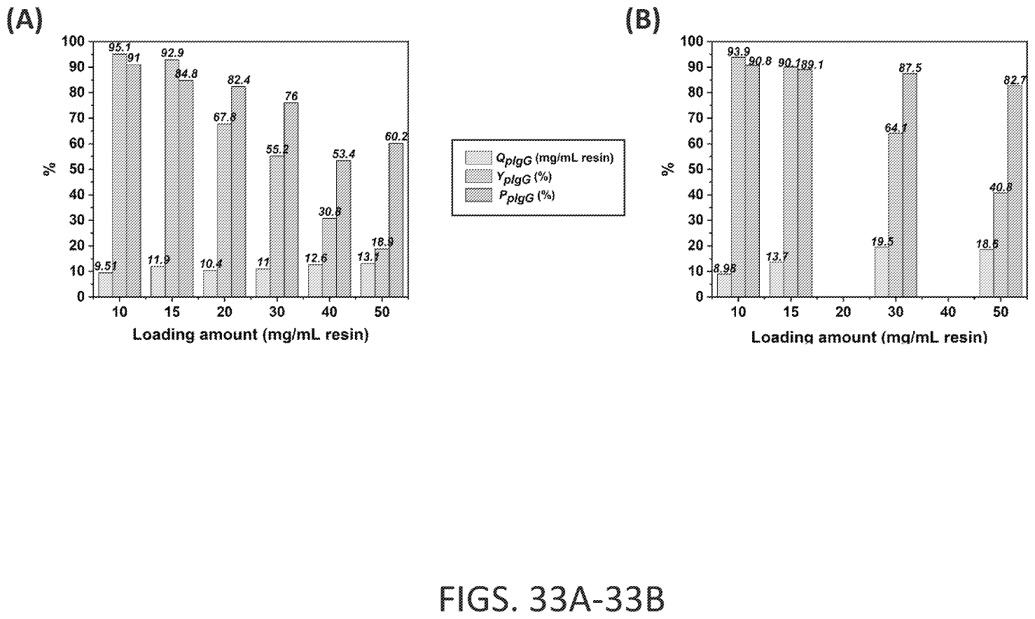

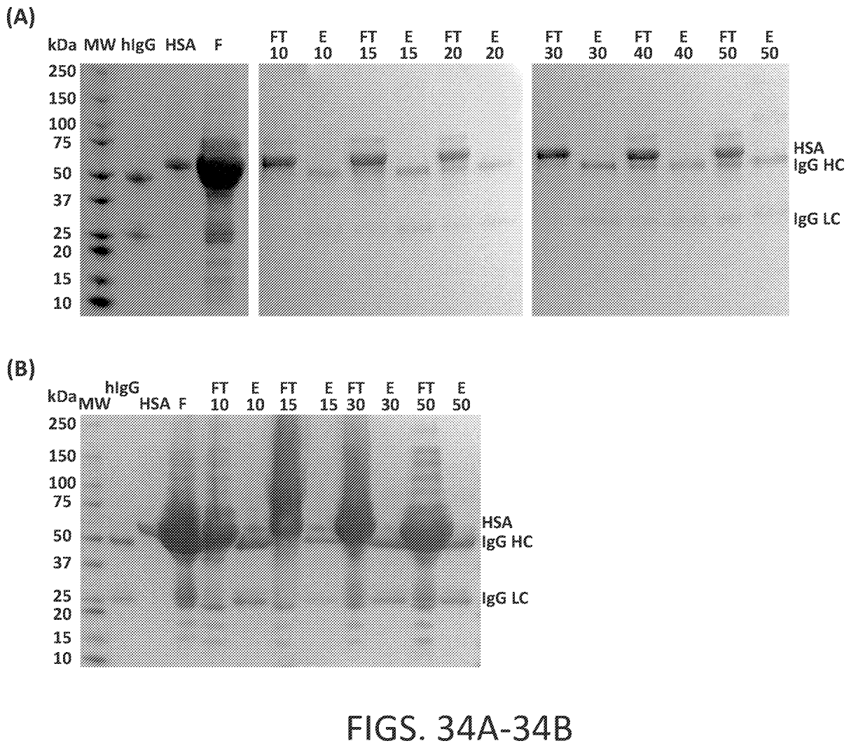

Following on the results discussed above, the application of LigaTrap™ resin was extended to purify pIgG from human cryoprecipitate “Cryo-rich” plasma. This feedstock is highly complex, as it contains, besides IgG (6.0-16 mg/mL, ˜150 kDa) and albumin (35-55 mg/mL, ˜69 kDa), substantial amounts of clotting factors including fibrinogen (factor I, 15-17 mg/ml, ˜340 kDa, pI˜5.8, and GRAVY index ˜−0.577), factor VIII (8-10 U/mL, ˜267 kDa, pI˜7.37), factor XIII (˜5 U/mL, ˜320 kDa, pI˜5.2), von Willebrand factor (VWF, ˜10 U/mL, ˜200 kDa, pI˜5.8), a nd fibronectin (220 μg/mL, ˜272 kDa, pI˜5.39), which vary widely by size and physicochemical properties. The selected loading and washing buffer, namely 0.5 M NaCl and 50 mM sodium caprylate in PBS at pH 7.4, was used to dilute the Cryo-rich plasma to a pIgG concentration of ˜7.0 mg/mL. To evaluate the robustness of the LigaTrap™ adsorbent against protein loading, different amounts of total proteins were loaded on the column, namely 1.0, 1.3, 1.9, 3.0, 4.2, and 5.3 mg, corresponding to 10, 15, 20, 30, 40 and 50 mg of IgG per mL of resin, respectively. The values of pIgG binding, yield, and purity determined by analyzing the eluted fractions via analytical Protein G HPLC, SEC HPLC, and SDS-PAGE are reported in FIG. 33 and FIG. 34.

At the loading value of 10 mg of pIgG per mL resin, the product yield is as high as 95.1% and purity is 91.0%, corresponding to a 3-fold enrichment compared to the feedstock, thus further corroborating the selectivity of LigaTrap™ adsorbent towards pIgG. Increasing the load, however, decreased significantly the product yield, which dropped from 93% at 15 mg per mL of resin to 19% at 50 mg per mL; concurrently, the purity of pIgG in the elution also decreased from 90% to 60%. Together, these results suggest that, as the load increase, the overwhelming amount of non-Ig plasma protein loaded on the column can displace the pIgG from the peptoid ligand, thus lowering both yield and purity, as evidenced in FIG. 34A. Additionally, following the formation of a first layer of pIgG molecules adsorbed by the affinity ligands on the surface of the chromatographic resin, ulterior layers of plasma proteins can be formed via non-specific—namely, electrostatic and hydrophobic—interactions driven by the high titer in the feedstock; the acidic elution environment triggers the dissociation of these non-Ig plasma proteins, which co-elute with pIgG and lower the purity of the eluted fraction while the yield is still high. The electrophoretic analysis of the collected chromatographic fractions (FIG. 34A) corroborates this interpretation, revealing the growing presence of abundant plasma proteins—e.g., albumin, fibrinogen, and α-2-macroglobulin—in the elution fractions obtained at higher values of protein load. Most notably, the control Protein G-agarose resin afforded comparable values of yield and purity, as shown in FIG. 33B and FIG. 34B, further demonstrating the value of LigaTrap™ resins as cost-effective alternative to traditional affinity adsorbents for industrial separations.

### Example 9

Purification of pIgG from cryo-rich plasma in flow-through mode using LigaGuard™ resin. FIG. 36 reports the profiles of pIgG breakthrough ratio, namely the ratio of pIgG titer in the effluent vs. feedstock (C_{pIgG}/C_{pIgG}*), and binding (Q_{pIgG}) vs. loading volume, while FIG. 37 report the breakthrough ratio of non-Ig plasma proteins (C_{pIgG}/C_{pIgG}*) vs. loading volume obtained using first-generation LigaGuard™. FIG. 38 report the profiles of pIgG breakthrough ratio (C_{pIgG}/C_{pIgG}*) and binding (Q_{pIgG}) vs. loading volume, while FIG. 39 report the corresponding profiles (C_{PP}/C_{PP}*) and (Q_{PP}) vs. loading volume for non-Ig plasma proteins obtained using second-generation LigaGuard™.

### Example 10

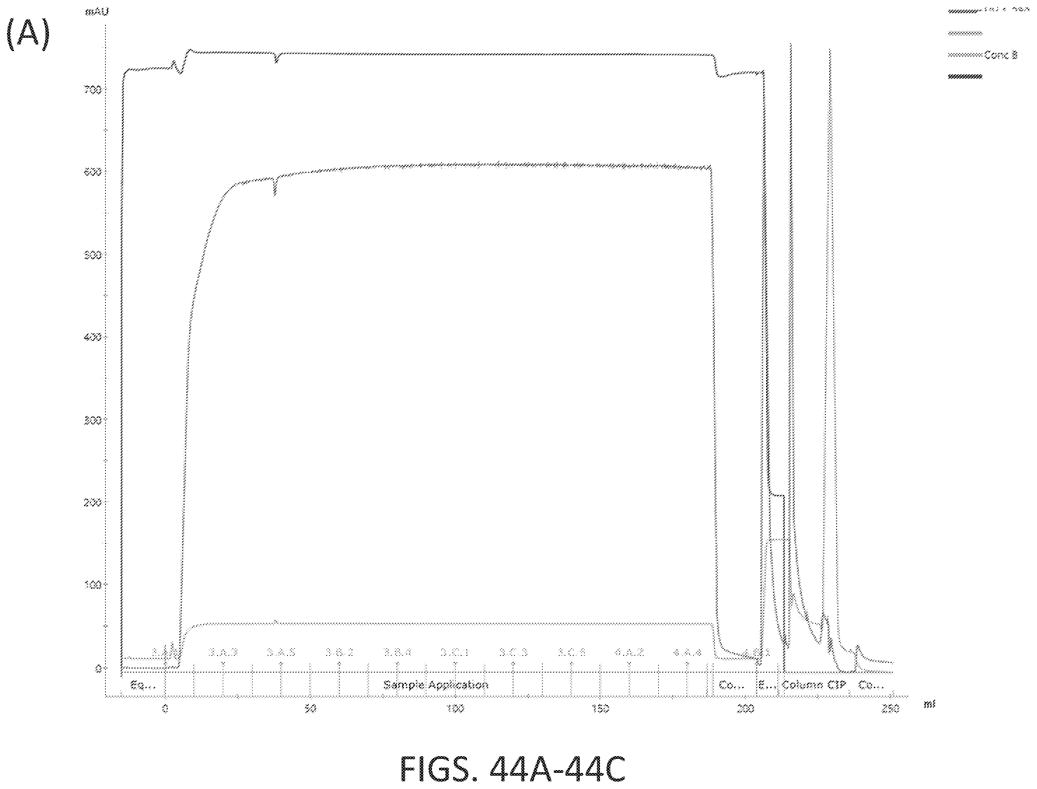

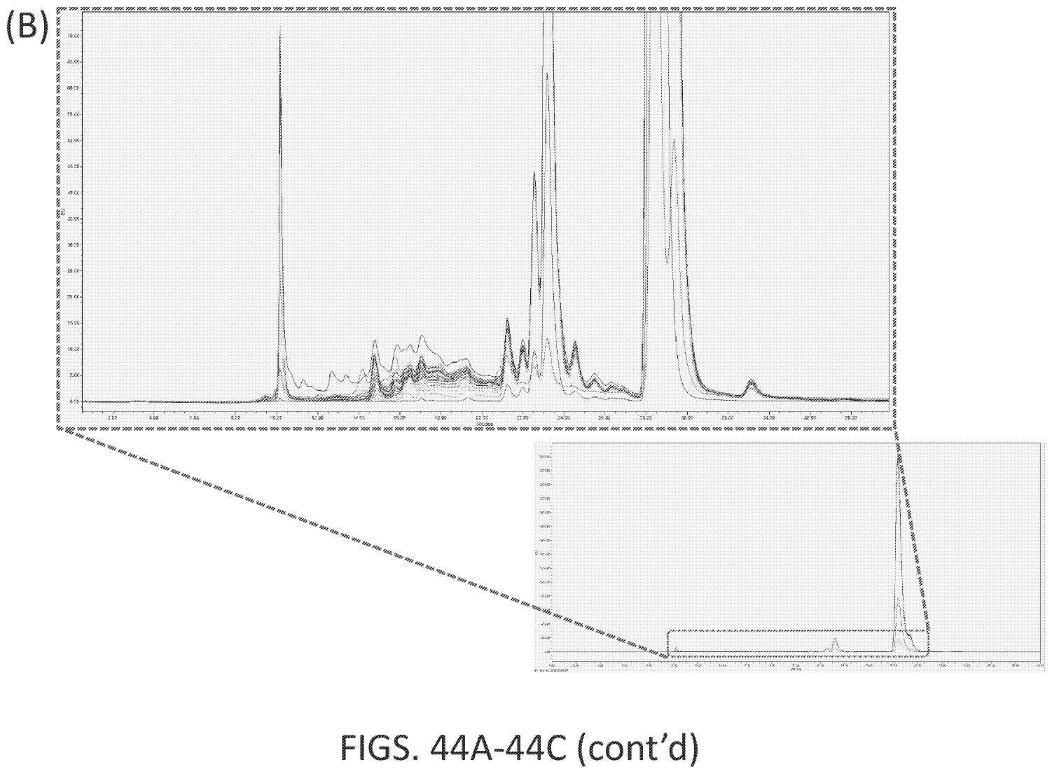

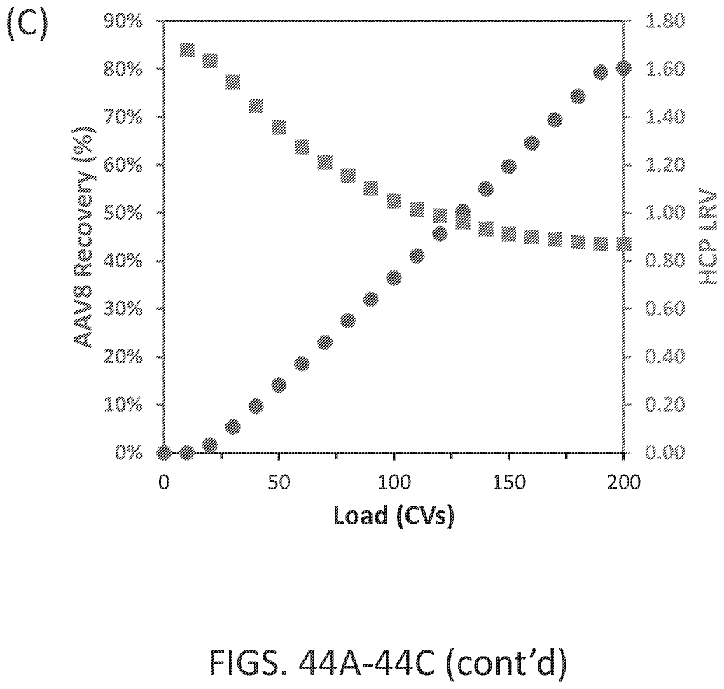

AAV8 purification via “flow-through” affinity chromatography. Purification studies of AAV8 from a clarified HEK293 cell lysate (clarified harvest) was performed in flow-through mode using G.2 LigaGuard™ resin packed in 1.5 mL chromatography column using an ÄKTA pure (Cytiva, Chicago, IL, USA) while continuously monitoring the effluents using UV spectroscopy at 280 nm. The resins were packed as a slurry in 20% v/v methanol in water and equilibrated with 20 mM NaCl ad 0.1% v/v Pluronic F-68 in 10 mM Bis-Tris, 20 mM NaCl buffer at pH 7.0 at 1.5 mL/min for 10 minutes. A volume of 200 mL of harvest was then loaded on each column at the residence time (RT) of 1.5 minutes and flow-through fractions of 15 mL (10 column volumes, CVs) were collected throughout the load and final column wash for analytical characterization (FIG. 44A). Column wash was performed with 10 CVs of 10 mM Bis-Tris buffer, 20 mM NaCl at pH 7.0 at 1 mL/min. Finally, a cleaning in place of the column was performed with 5 CVs of 150 mM Phosphoric acid (containing 0.1% v/v Pluronic-F68) followed by 5 CVs of 6 N Guanidine (containing 0.1% v/v Pluronic-F68).

Analytical size exclusion chromatography-ultra-high pressure liquid chromatography (SEC-UPLC) for high throughput mAb purity estimation. The flow-through fractions were analyzed for molecular weight distribution using a BioResolve SEC mAb Column, using 200 mM KCl and 0.05-0.1% NaN₃ in 50 mM sodium phosphate buffer at pH 7 as mobile phase (FIG. 44B). A sample volume of 10 μL was injected at the flowrate of 0.5 mL/min and the UV absorbance of the effluent was continuously monitored via UV spectroscopy at the wavelengths of 260 nm and 280 nm as well as fluorescence spectroscopy (ex./em. 280/350 nm). The resulting chromatograms were divided into (i) AAV product (retention time: 10⁻¹¹ min), HEK293 HCPs (retention time: 8-22 min), and media components (retention time: 22-34 min). The corresponding peak areas were utilized to estimate the values of AAV titer using a calibration curve. The titer of AAV in the effluent was also measured using anti-AAV8 ELISA kit (PROGEN Biotechnik GmbH); similarly, the titer of HEK293 host cell proteins (HCPs) in the effluent was measured using anti-HEK293 HCP ELISA kit (Cygnus) (FIG. 44C).

### Example 11

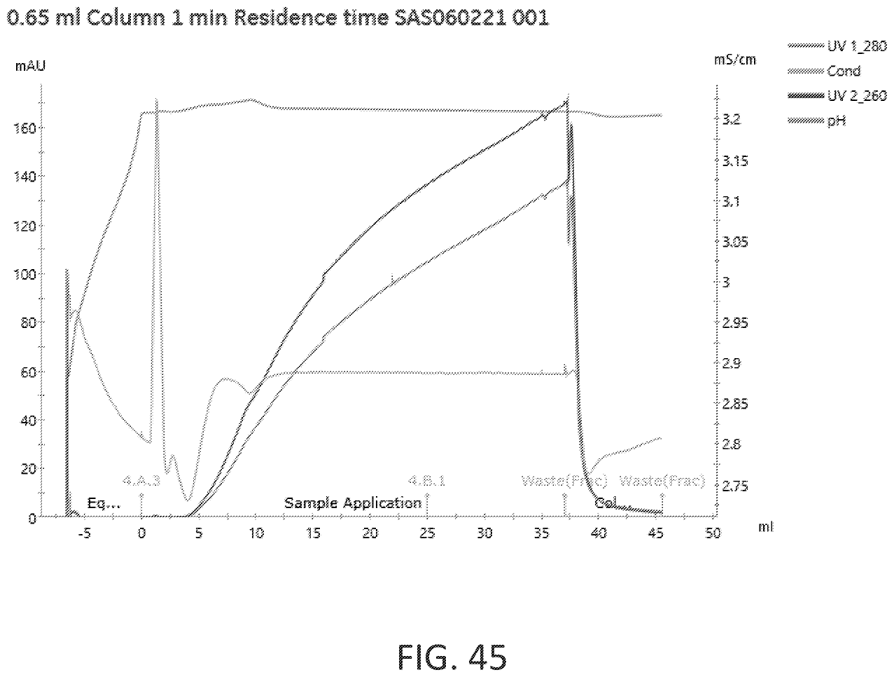

AAV2 purification via flow-through affinity chromatography. Purification studies of AAV2 from a clarified HEK293 cell lysate (clarified harvest) was performed in flow-through mode using G.2 LigaGuard™ resin packed in 0.65 mL chromatography column using an ÄKTA pure (Cytiva, Chicago, IL, USA) while continuously monitoring the effluents using UV spectroscopy at 280 nm (FIG. 45). The resins were packed as a slurry in 20% v/v methanol in water and equilibrated with 20 mM NaCl ad 0.1% v/v Pluronic F-68 in 10 mM Bis-Tris, 20 mM NaCl buffer at pH 7.0 at 0.65 mL/min for 10 minutes. A volume of 35 mL of harvest was then loaded on each column at the residence time (RT) of 1.0 minutes and two flow-through fractions of 25 mL (38.5 column volumes, CVs) and 12 mL were collected throughout the load and final column wash for analytical characterization. Column wash was performed with 10 CVs of 10 mM Bis-Tris buffer, 20 mM NaCl at pH 7.0 at 1 mL/min. Finally, a cleaning in place of the column was performed with 5 CVs of 150 mM Phosphoric acid (containing 0.1% v/v Pluronic-F68) followed by 5 CVs of 6 N Guanidine (containing 0.1% v/v Pluronic-F68). Recovery metrics of the AAV2 capsids using flow-through mode are provided in Table 6 below.

TABLE 6
Recovery metrics of the AAV2 capsids using flow-through mode.

| Sample Name | Volume | Total AAV2 Capsids | Recovery % |
|---|---|---|---|
| Load | 35 | 1.24E+13 | |
| Flow-through 1 | 25 | 4.47E+12 | 36.05 |
| Flow-through 2 | 12 | 3.96E+12 | 31.94 |
| Wash | 10 | 6.70E+11 | 5.40 |
| | | Total % Recovery: | 73.39 |

### Example 12

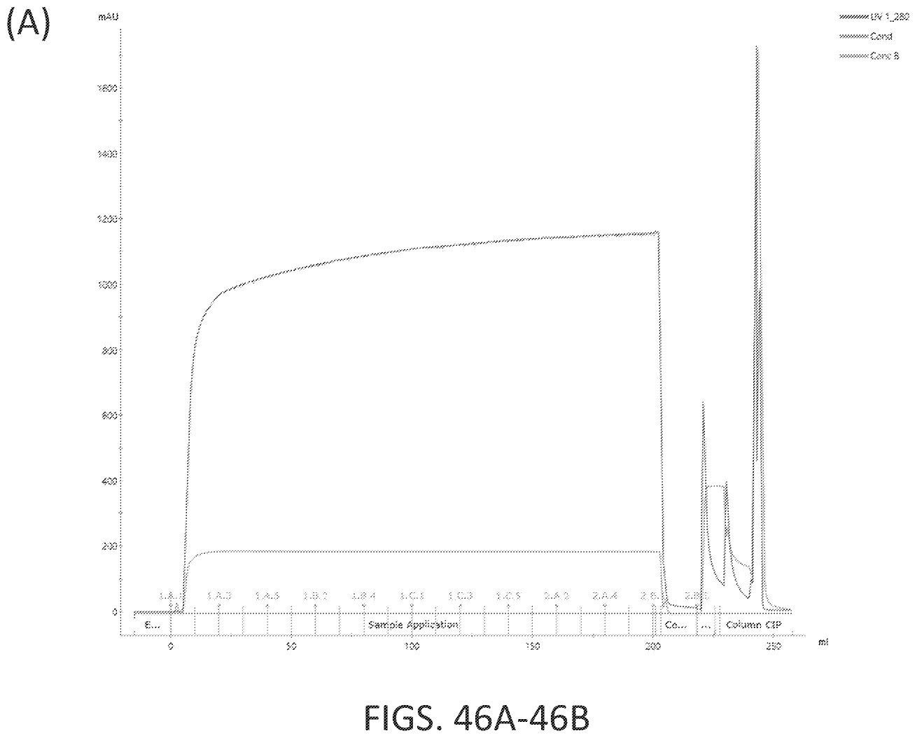

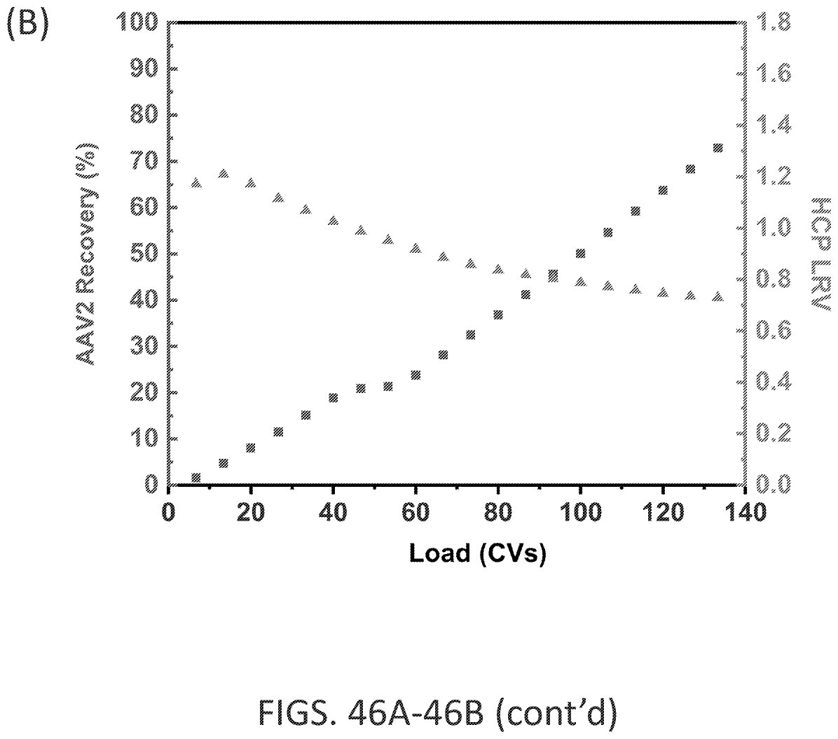

AAV2 purification via flow-through mode using LigaGuard™ resin. Purification studies of AAV2 from a clarified HEK293 cell lysate (clarified harvest) was performed in flow-through mode using G.2 LigaGuard™ resin packed in 1.5 mL chromatography column using an ÄKTA avant 150 (Cytiva, Chicago, IL, USA) while continuously monitoring the effluents using UV spectroscopy at 280 nm. The resins were packed as a slurry in 20% v/v methanol in water and equilibrated with 20 mM NaCl ad 0.1% v/v Pluronic F-68 in 10 mM Bis-Tris, 20 mM NaCl buffer at pH 7.0 at 1.5 mL/min for 10 minutes. A volume of 200 mL of harvest was then loaded on each column at the residence time (RT) of 1.5 minutes and flow-through fractions of 10 mL (6.7 column volumes, CVs) were collected throughout the load and final column wash for analytical characterization (FIG. 46A). Column wash was performed with 10 CVs of 10 mM Bis-Tris buffer, 20 mM NaCl at pH 7.0 at 1 mL/min. Finally, a cleaning in place of the column was performed with 5 CVs of 150 mM Phosphoric acid (containing 0.1% v/v Pluronic-F68) followed by 5 CVs of 6 N Guanidine (containing 0.1% v/v Pluronic-F68).

Analytical size exclusion chromatography-ultra-high pressure liquid chromatography (SEC-UPLC) for high throughput AAV titration estimation. The flow-through fractions were analyzed for molecular weight distribution using a BioResolve SEC mAb Column, using 200 mM KCl and 0.05-0.1% NaN₃ in 50 mM sodium phosphate buffer at pH 7 as mobile phase. A sample volume of 10 μL was injected at the flowrate of 0.5 mL/min and the UV absorbance of the effluent was continuously monitored via UV spectroscopy at the wavelengths of 260 nm and 280 nm as well as fluorescence spectroscopy (ex./em. 280/350 nm). The resulting chromatograms were divided into (i) AAV product (retention time: 10⁻¹¹ min), HEK293 HCPs (retention time: 8-22 min), and media components (retention time: 22-34 min). The corresponding peak areas were utilized to estimate the values of AAV titer using a calibration curve. The titer of AAV in the effluent was also measured using anti-AAV2 ELISA kit (PROGEN Biotechnik GmbH); similarly, the titer of HEK293 host cell proteins (HCPs) in the effluent was measured using anti-HEK293 HCP ELISA kit (Cygnus) (FIG. 46B).

### Example 13

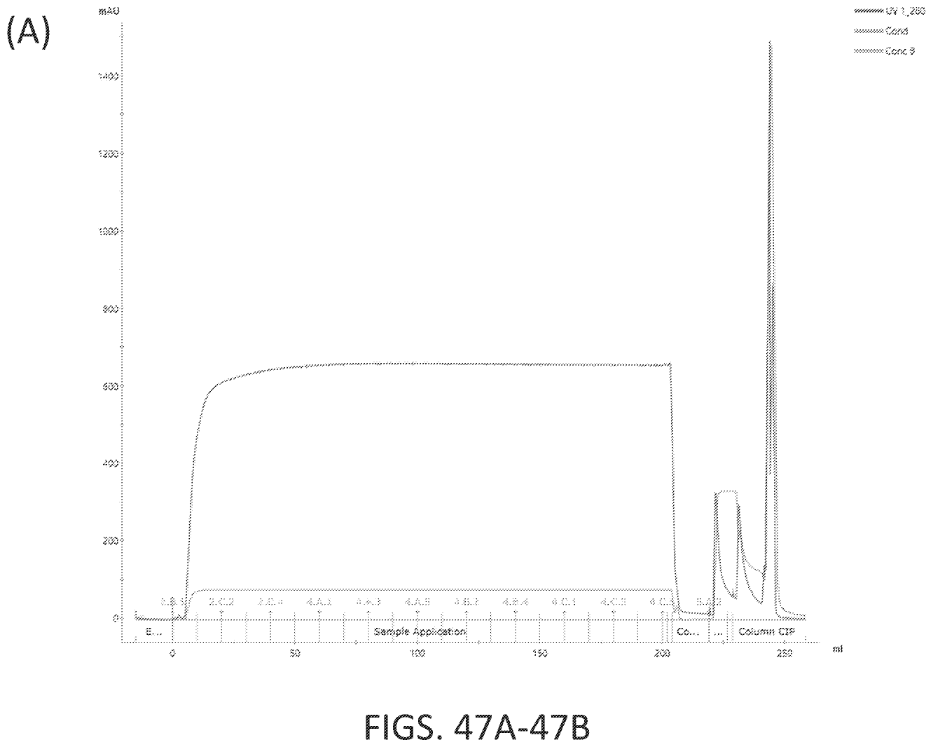

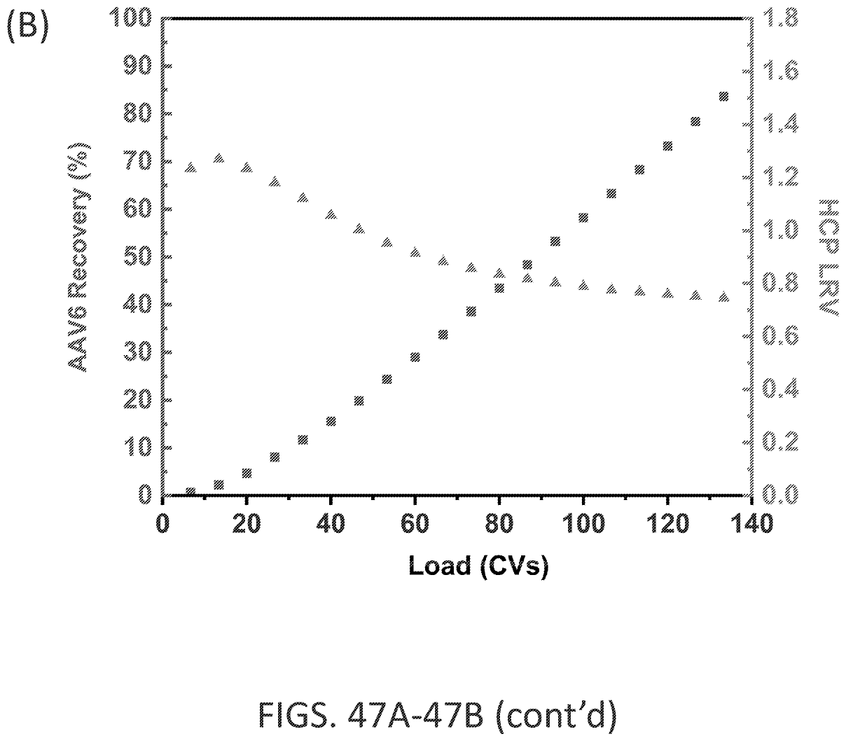

AAV6 purification via flow-through mode using LigaGuard™ resin. Purification studies of AAV6 from a clarified HEK293 cell lysate (clarified harvest) was performed in flow-through mode using G.2 LigaGuard™ resin packed in 1.5 mL chromatography column using an ÄKTA avant 150 (Cytiva, Chicago, IL, USA) while continuously monitoring the effluents using UV spectroscopy at 280 nm. The resins were packed as a slurry in 20% v/v methanol in water and equilibrated with 20 mM NaCl ad 0.1% v/v Pluronic F-68 in 10 mM Bis-Tris, 20 mM NaCl buffer at pH 7.0 at 1.5 mL/min for 10 minutes. A volume of 200 mL of harvest was then loaded on each column at the residence time (RT) of 1.5 minutes and flow-through fractions of 10 mL (6.7 column volumes, CVs) were collected throughout the load and final column wash for analytical characterization (FIG. 47A). Column wash was performed with 10 CVs of 10 mM Bis-Tris buffer, 20 mM NaCl at pH 7.0 at 1 mL/min. Finally, a cleaning in place of the column was performed with 5 CVs of 150 mM Phosphoric acid (containing 0.1% v/v Pluronic-F68) followed by 5 CVs of 6 N Guanidine (containing 0.1% v/v Pluronic-F68).

Analytical size exclusion chromatography-ultra-high pressure liquid chromatography (SEC-UPLC) for high throughput AAV titration estimation. The flow-through fractions were analyzed for molecular weight distribution using a BioResolve SEC mAb Column, using 200 mM KCl and 0.05-0.1% NaN₃ in 50 mM sodium phosphate buffer at pH 7 as mobile phase. A sample volume of 10 μL was injected at the flowrate of 0.5 mL/min and the UV absorbance of the effluent was continuously monitored via UV spectroscopy at the wavelengths of 260 nm and 280 nm as well as fluorescence spectroscopy (ex./em. 280/350 nm). The resulting chromatograms were divided into (i) AAV product (retention time: 10⁻¹¹ min), HEK293 HCPs (retention time: 8-22 min), and media components (retention time: 22-34 min). The corresponding peak areas were utilized to estimate the values of AAV titer using a calibration curve. The titer of AAV in the effluent was also measured using anti-AAV2 ELISA kit (PROGEN Biotechnik GmbH); similarly, the titer of HEK293 host cell proteins (HCPs) in the effluent was measured using anti-HEK293 HCP ELISA kit (Cygnus) (FIG. 47B).

### Example 14

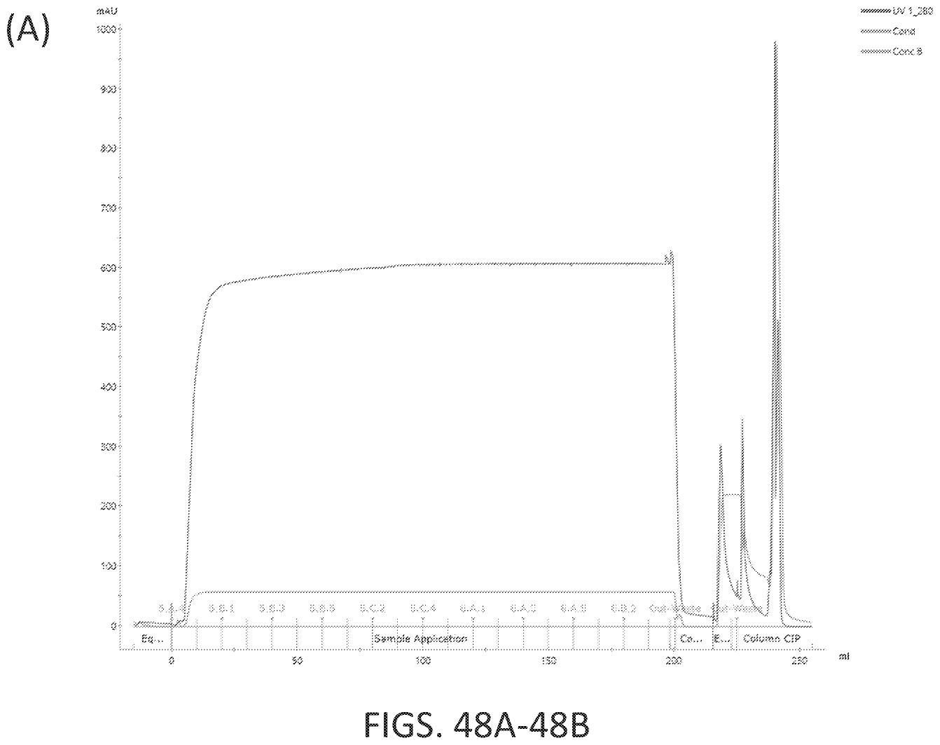

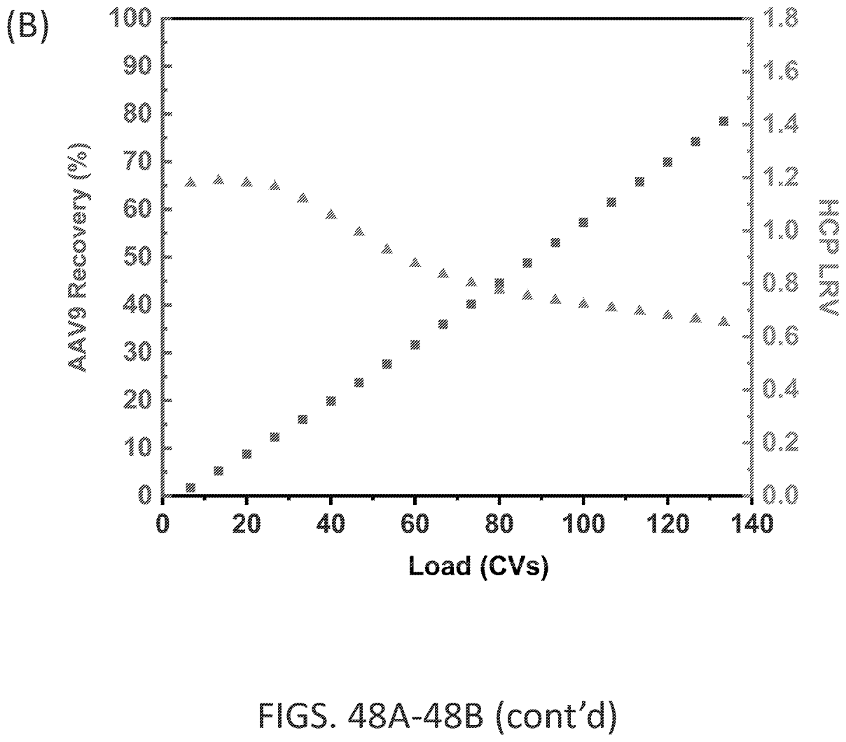

AAV9 purification via flow-through mode using LigaGuard™ resin. Purification studies of AAV9 from a clarified HEK293 cell lysate (clarified harvest) was performed in flow-through mode using G.2 LigaGuard™ resin packed in 1.5 mL chromatography column using an ÄKTA avant 150 (Cytiva, Chicago, IL, USA) while continuously monitoring the effluents using UV spectroscopy at 280 nm. The resins were packed as a slurry in 20% v/v methanol in water and equilibrated with 20 mM NaCl ad 0.1% v/v Pluronic F-68 in 10 mM Bis-Tris, 20 mM NaCl buffer at pH 7.0 at 1.5 mL/min for 10 minutes. A volume of 200 mL of harvest was then loaded on each column at the residence time (RT) of 1.5 minutes and flow-through fractions of 10 mL (6.7 column volumes, CVs) were collected throughout the load and final column wash for analytical characterization (FIG. 48A). Column wash was performed with 10 CVs of 10 mM Bis-Tris buffer, 20 mM NaCl at pH 7.0 at 1 mL/min. Finally, a cleaning in place of the column was performed with 5 CVs of 150 mM Phosphoric acid (containing 0.1% v/v Pluronic-F68) followed by 5 CVs of 6 N Guanidine (containing 0.1% v/v Pluronic-F68).

Analytical size exclusion chromatography-ultra-high pressure liquid chromatography (SEC-UPLC) for high throughput AAV titration estimation. The flow-through fractions were analyzed for molecular weight distribution using a BioResolve SEC mAb Column, using 200 mM KCl and 0.05-0.1% NaN₃ in 50 mM sodium phosphate buffer at pH 7 as mobile phase. A sample volume of 10 μL was injected at the flowrate of 0.5 mL/min and the UV absorbance of the effluent was continuously monitored via UV spectroscopy at the wavelengths of 260 nm and 280 nm as well as fluorescence spectroscopy (ex./em. 280/350 nm). The resulting chromatograms were divided into (i) AAV product (retention time: 10⁻¹¹ min), HEK293 HCPs (retention time: 8-22 min), and media components (retention time: 22-34 min). The corresponding peak areas were utilized to estimate the values of AAV titer using a calibration curve. The titer of AAV in the effluent was also measured using anti-AAV2 ELISA kit (PROGEN Biotechnik GmbH); similarly, the titer of HEK293 host cell proteins (HCPs) in the effluent was measured using anti-HEK293 HCP ELISA kit (Cygnus) (FIG. 48B).

### Example 15

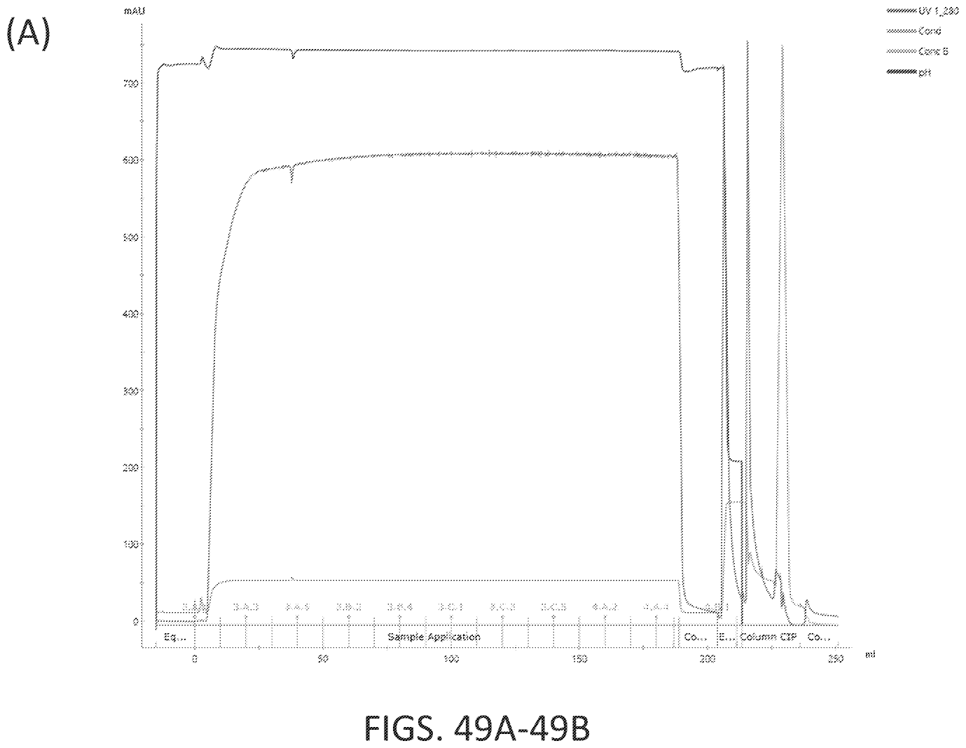

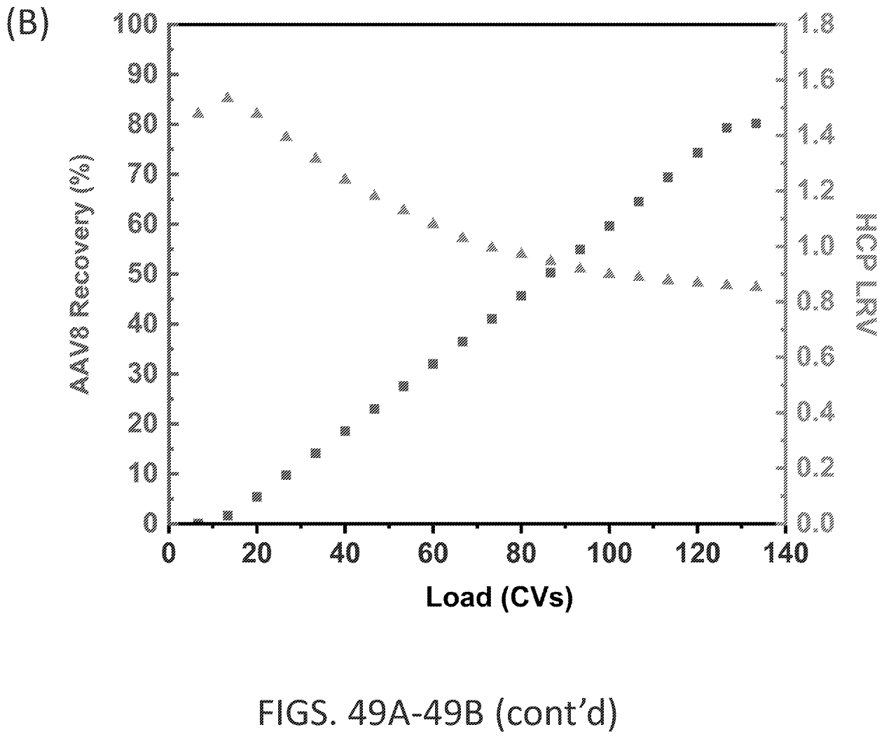

AAV8 purification via flow-through mode using LigaGuard™ resin. Purification studies of AAV8 from a clarified HEK293 cell lysate (clarified harvest) was performed in flow-through mode using G.2 LigaGuard™ resin packed in 1.5 mL chromatography column using an ÄKTA avant 150 (Cytiva, Chicago, IL, USA) while continuously monitoring the effluents using UV spectroscopy at 280 nm. The resins were packed as a slurry in 20% v/v methanol in water and equilibrated with 20 mM NaCl ad 0.1% v/v Pluronic F-68 in 10 mM Bis-Tris, 20 mM NaCl buffer at pH 7.0 at 1.5 mL/min for 10 minutes. A volume of 200 mL of harvest was then loaded on each column at the residence time (RT) of 1.5 minutes and flow-through fractions of 10 mL (6.7 column volumes, CVs) were collected throughout the load and final column wash for analytical characterization (FIG. 49A). Column wash was performed with 10 CVs of 10 mM Bis-Tris buffer, 20 mM NaCl at pH 7.0 at 1 mL/min. Finally, a cleaning in place of the column was performed with 5 CVs of 150 mM Phosphoric acid (containing 0.1% v/v Pluronic-F68) followed by 5 CVs of 6 N Guanidine (containing 0.1% v/v Pluronic-F68).

Analytical size exclusion chromatography-ultra-high pressure liquid chromatography (SEC-UPLC) for high throughput AAV titration estimation. The flow-through fractions were analyzed for molecular weight distribution using a BioResolve SEC mAb Column, using 200 mM KCl and 0.05-0.1% NaN₃ in 50 mM sodium phosphate buffer at pH 7 as mobile phase. A sample volume of 10 μL was injected at the flowrate of 0.5 mL/min and the UV absorbance of the effluent was continuously monitored via UV spectroscopy at the wavelengths of 260 nm and 280 nm as well as fluorescence spectroscopy (ex./em. 280/350 nm). The resulting chromatograms were divided into (i) AAV product (retention time: 10⁻¹¹ min), HEK293 HCPs (retention time: 8-22 min), and media components (retention time: 22-34 min). The corresponding peak areas were utilized to estimate the values of AAV titer using a calibration curve. The titer of AAV in the effluent was also measured using anti-AAV2 ELISA kit (PROGEN Biotechnik GmbH); similarly, the titer of HEK293 host cell proteins (HCPs) in the effluent was measured using anti-HEK293 HCP ELISA kit (Cygnus) (FIG. 49B).

### 4. Materials and Methods

Fmoc-protected amino acids Fmoc-Gly-OH, Fmoc-Ser(tBu)-OH, Fmoc-Ile-OH, Fmoc-Ala-OH, Fmoc-Phe-OH, Fmoc-Tyr(tBu)-OH, Fmoc-Asp(OtBu)-OH, Fmoc-His(Trt)-OH, Fmoc-Arg(Pbf)-OH, Fmoc-Lys(Boc)-OH, Fmoc-Asn(Trt)-OH, Fmoc-Glu(OtBu)-OH, Fmoc-Pro-OH, Fmoc-Trp(Boc)-OH, Fmoc-Cys(Trt)-OH, and Fmoc-Leu-OH, the coupling agent Azabenzotriazole Tetramethyl Uronium Hexafluorophosphate (HATU), and diisopropylethylamine (DIPEA), piperidine, and trifluoroacetic acid (TFA) were sourced from ChemImpex International (Wood Dale, IL, USA). The Toyopearl AF-Amino-650M resin was obtained from Tosoh Bioscience (Tokyo, Japan). Triisopropylsilane (TIPS), 1,2-ethanedithiol (EDT), anisole, Kaiser test kits, NISTmAb and Protein G Sepharose® Fast Flow resin were from MilliporeSigma (St. Louis, MO, USA). N,N′-dimethylformamide (DMF), dichloromethane (DCM), methanol, and N-methyl-2-pyrrolidone (NMP), sodium phosphate (monobasic), sodium phosphate (dibasic), hydrochloric acid, glycine, Bis-Tris, and Bicinchoninic acid (BCA) assay were obtained from Fisher Chemicals (Hampton, NH, USA). Harvested CHO cell culture harvests (HCCF) containing monoclonal antibodies were generously provided by Genentech (San Francisco, CA) and Merck (Kenilworth, NJ); the values of mAb and HCP titer are reported in Table 7. Vici Jour PEEK 2.1 mm ID, 30 mm empty chromatography columns and 10 μm polyethylene frits were obtained from VWR International (Radnor, PA, USA). The Yarra 3 μm SEC-2000 300×7.8 mm size exclusion chromatography column was obtained from Phenomenex Inc. (Torrance, CA, USA). CHO-specific HCP ELISA kits were obtained from Cygnus Technologies (Southport, NC).

TABLE 7
mAb titer and properties and HCP titers in the CHO
cell culture harvests utilized in this study.

| | | mAb | |
|---|---|---|---|
| HCCF | mAb titer | subclass and pI | HCP titer |
| 1 | 4.05 mg/mL | IgG₄ \| 7.3 | 0.596 mg/mL |
| 2 | 8.56 mg/mL | Proprietary | 0.421 mg/mL |
| 3 | 1.09 mg/mL | Proprietary | 0.419 mg/mL |
| 4 | 6.65 mg/mL | IgG₁ \| 9.2 | 0.572 mg/mL |
| 5 | 5.04 mg/mL | IgG₁ \| 8.7 | 0.283 mg/mL |
| 6 | 0.72 mg/mL | Proprietary | 0.261 mg/mL |

Preparation of LigaGuard™ resin. The peptide-based G.1 and G.2 LigaGuard™ resin were prepared via direct peptide synthesis on Toyopearl AF-Amino-650M resin via Fmoc/tBu strategy as described in prior work (note: the values of peptide density on Toyopearl resin are proprietary information of LigaTrap Technologies LLC) and stored in 20% v/v aqueous methanol for long-term storage.

Static Binding Studies. Static binding studies were performed on G.1 LigaGuard™ and G.2 resin using null CHO-S cell culture harvest donated by BTEC at NC State University. Briefly, 50 μL of LigaGuard™ resin were incubated with 200 μL of either CHO harvest or NISTmAb or a combination of both at different titers (0.05-2 mg/mL), for 2.5 hours under gentle agitation. Following centrifugation of the resin, the supernatant was analyzed via BCA assay to measure the bound, equilibrium HCP or mAb concentration when testing individual species. For studies with the simulated HCCF (NISTmAb+CHO HCP), a CHO HCP ELISA assay obtained from Cygnus Technologies (Southport, SC) was used to quantify and CHO HCP concentrations. The mass of protein adsorbed per volume of resin was calculated via mass balance. The adsorption data were fit against a Langmuir isotherm to calculate the values of maximum binding capacity at equilibrium (Qₘₐₓ) and affinity (i.e., dissociation constant, K_{D}).

mAb purification via “flow-through” affinity chromatography. Purification studies of therapeutic mAbs from the industrial CHO cell culture harvests listed in Table 7 were performed in flow-through mode using G.1 and G.2 LigaGuard™ resin packed in 0.1 mL chromatography columns. The resins were packed as a slurry in 20% v/v methanol in water and equilibrated with 20 mM Bis-Tris buffer at pH 6.5 at 0.2 mL/min for 10 minutes. A volume of 10 mL of harvest was then loaded on each column at the residence time of 1 minute or 2 minutes (for ELISA) and flow-through fractions of 1 mL were collected throughout the load and final column wash for analytical characterization. All purification studies were performed using an ÄKTA pure (Cytiva, Chicago, IL, USA) while monitoring the effluents using UV spectroscopy at 280 nm.

mAb quantification using analytical Protein A Chromatography. The mAb concentration in the CHO cell culture harvests and the flow-through fractions generated using G.1 and G.2 LigaGuard™ resin was measured via analytical Protein G chromatography using a 0.1 mL Protein G Sepharose Fast Flow column installed on a Waters Alliance 2690 system equipped with a Waters 2487 dual absorbance detector (Waters Corporation, Milford, MA, USA). A calibration curve was initially constructed using pure NISTmAb in PBS at pH 7.4 at the concentrations of 0.1, 0.5, 1.0, 2.0, 4.0, 6.0, 8.0, and 10.0 mg/mL. A volume of 20 μL of either a calibration sample or a flow-through fraction was injected onto the Protein G column at 0.5 mL/min and elution was performed with 0.1 M Glycine HCl at pH 2.5 at the same flowrate. UV absorbance of the eluate was continuously monitored at 280 nm and the resulting chromatograms were utilized to calculate the cumulative and fractional yields as described in prior work.

Analytical Size Exclusion Chromatography (SEC) for high throughput mAb purity estimation. The flow-through fractions were analyzed for molecular weight distribution using a Yarra 3 μm SEC-2000 column with PBS at pH 7.4 as mobile phase. A sample volume of 50 μL was injected at the flowrate of 0.5 mL/min and the UV absorbance of the effluent was continuously monitored at 280 nm. The resulting chromatograms were divided into (i) high molecular weight peak segment (MW>150 kDa), mAb product peak segment (MW˜150 kDa), and low molecular weight peak segment (10 kDa<MW<150 kDa) based on retention time. The corresponding peak areas were utilized to estimate the values of fractional and cumulative mAb purity as described in prior work.

Measurement of HCP LRV via CHO-specific Enzyme-Linked Immunosorbent Assay (ELISA). Selected flow-through fractions were also analyzed using CHO-specific ELISA kits obtained from Cygnus Technologies (Southport, SC) to measure the values of fractional and cumulative HCP LRV in the effluents produced using both G.1 and G.2 LigaGuard™ resin.

Proteomic analysis via liquid chromatography tandem mass spectrometry (LC-MS-MS). The CHO HCCFs and flow-through fractions were analyzed following the proteomics protocol described in prior work. Briefly, the samples were initially digested using a FASP protocol adapted from Wiśniewski et al., and analyzed using a nano LC-MS-MS instrument at the Molecular Education, Technology, and Research Innovation Center (METRIC) at NC State University. Post-acquisition data analysis was performed using Proteome Discoverer 2.2 (Thermo Fisher, San Jose, CA) against a Cricetulus griseus (Chinese hamster) CHO genome/EMBL database. The relative quantification of individual HCPs in the flow-through samples and the corresponding values of % removal were calculated as described by Lavoie et al. Finally, “Captured HCPs” were defined as (i) proteins identified in the cell culture but not in the flow-through effluent (note: “identified”, significant species are the ones with a sum of >4 spectral counts of their fragments) or (ii) are compared to be present in (statistically) significantly lower concentrations than in the feedstock, computed by ANOVA.

Materials. The Fmoc-Cys-(Trt)-Rink polystyrene resin was purchased from Anaspec (Fremont, CA, USA), Toyopearl AF-Amino-650M resin was obtained from Tosoh Corporation (Tokyo, Japan), and WorkBeads 40 ACT resin was from Bioworks (Uppsala, Sweden). Fmoc-N-[3-(N-Pbf-guanidino)-propyl]-glycine was from PolyPeptide (Torrance, CA, USA). Fluorenylmethoxycarbonyl-(Fmoc-) protected amino acids Fmoc-Gly-OH, Fmoc-Ser(tBu)-OH, Fmoc-Ile-OH, Fmoc-Ala-OH, Fmoc-Phe-OH, Fmoc-Tyr(tBu)-OH, Fmoc-Asp(OtBu)-OH, Fmoc-His(Trt)-OH, Fmoc-Arg(Pbf)-OH, Fmoc-Lys(Boc)-OH, Fmoc-Asn(Trt)-OH, and Fmoc-Glu(OtBu)-OH, Hexafluorophosphate Azabenzotriazole Tetramethyl Uronium (HATU), diisopropylethylamine (DIPEA), piperidine, and trifluoroacetic acid (TFA) were obtained from ChemImpex International (Wood Dale, IL, USA). Kaiser test kits, triisopropylsilane (TIPS), and 1,2-ethanedithiol (EDT) were obtained from Millipore Sigma (St. Louis, MO, USA). N,N′-dimethylformamide (DMF), dichloromethane (DCM), methanol, and N-methyl-2-pyrrolidone (NMP) were obtained from Fisher Chemical (Hampton, NH, USA).

Human polyclonal Immunoglobulin G (IgG) in lyophilized form was purchased from Athens Research & Technology, Inc (Athens, GA, USA). Ig-rich paste, and cryo-rich and cryo-poor human plasma were a gift of CSL Behring (King of Prussia, PA, USA). Sodium phosphate monobasic (NaH₂PO₄), sodium phosphate dibasic (Na₂HPO₄), hydrochloric acid, sodium hydroxide, Bis-Tris, ethanol, sodium chloride (NaCl), and sodium caprylate (NaCapr) were purchased from Fisher Scientific (Hampton, NH, USA). Phosphate buffered saline at pH 7.4 was purchased from Millipore Sigma (St. Louis, MO, USA). Vici Jour PEEK chromatography columns (2.1 mm ID, 30 mm length, 0.1 mL volume), Alltech chromatography columns (3.6 mm ID, 50 mm length, 0.5 mL volume), and 10 μm polyethylene frits were obtained from VWR International (Radnor, PA, USA). The Yarra 3 μm SEC-2000 300×7.8 mm size exclusion chromatography column was obtained from Phenomenex Inc. (Torrance, CA, USA). Protein G Sepharose™ Fast Flow resin was purchased from Millipore Sigma (Burlington, MA, USA). The 10⁻²⁰% Tris-Glycine HCl SDS-PAGE gels and Coomassie blue stain were purchased from Bio-Rad Life Sciences (Carlsbad, CA, USA). A Pierce™ BCA Protein Assay Kit was purchased from Fisher Scientific™ (Pittsburgh, PA, USA). All chromatographic experiments were performed using a Waters Alliance 2690 separations module system equipped with a Waters 2487 dual absorbance detector were purchased from Waters Corporation (Milford, MA, USA).

Preparation of LigaTrap™ Human IgG resin and LigaGuard™ resin. The peptoid ligand PL-16 was synthesized and conjugated to WorkBeads 40 ACT resin as described in prior work (note: the conjugation strategy and the value of peptoid density on WorkBeads resin are proprietary information of LigaTrap Technologies LLC)^{[32, 33]}. The resulting LigaTrap™ Human IgG resin was rinsed in water and stored in 20% v/v aqueous methanol for long-term storage. The peptide-based LigaGuard™ resin was produced by direct peptide synthesis on Toyopearl AF-Amino-650M resin via Fmoc/tBu strategy as described in prior work (note: the values of peptide density on Toyopearl resin are proprietary information of LigaTrap Technologies LLC)^{[34]}, and stored in 20% v/v aqueous methanol for long-term storage.

Dynamic binding capacity of pure IgG on LigaTrap™ Human IgG resin. The dynamic binding capacity of LigaTrap™ Human IgG resin at 10% breakthrough of IgG (DBC_{10%}, mg/mL resin) was measured as reported in prior studies. A volume of 0.1 mL of LigaTrap™ Human IgG resin was wet packed in a Vici Jour PEEK column, washed with 10 column volumes (CVs) of 20% v/v ethanol, deionized water (3 CVs), and finally equilibrated with 10 CVs of PBS buffer at pH 7.4. A volume of 2 mL of solution of human polyclonal IgG at either 5 mg/mL or 10 mg/mL in PBS buffer was continuously loaded on the column at the flow rate of either 0.05 mL/min (residence time, RT: 2 min) or 0.02 mL/min (RT: 5 min). Following load, the resin was washed with 10 CVs of PBS buffer at the flow rate of 0.1 mL/min. IgG elution was then performed with 20 CVs of 0.2 M acetate buffer at pH 4.0 at the flow rate of 0.2 mL/min. The resin was regenerated with 10 CVs of 0.1 M glycine buffer at pH 2.5 at the flow rate of 0.2 mL/min. The effluents were continuously monitored by UV spectrometry at 280 nm and the resulting chromatograms were utilized to calculate the DBC_{10%} of IgG.

Purification of IgG from Ig-rich paste, cryo-poor, and cryo-rich human plasma using LigaTrap™ Human IgG resin in bind-and-elute mode. A volume of 0.1 mL of LigaTrap™ Human IgG resin was wet packed in a Vici Jour PEEK column, washed with 20% v/v ethanol (10 CVs), deionized water (3 CVs), and finally equilibrated with binding buffer (10 CVs) at the flow rate of 0.2 mL/min. The following binding buffers were prepared: (i) X M NaCl in PBS at pH Y, wherein X is either 0, 0.15, 0.25, or 0.5, or Y is either 6.5, 7.0, 7.4, or 8.0; and (ii) 0.5 M NaCl and Z mM NaCapr in PBS at pH Y, wherein Z is either 0, 25, 50, or 75, and Y is either 7.4 or 8.0. The Ig-rich paste was dissolved in the binding buffer to achieve a total protein concentration of ˜10 mg/mL by stirring the solution overnight at 4° C.; cryo-poor plasma was diluted in binding buffer to achieve a total protein titer of 25.7 mg/mL and an IgG titer of 7.4 mg/mL; similarly, cryo-rich plasma was diluted in binding buffer to achieve a total protein titer of 30.0 mg/mL and an IgG titer of 7.0 mg/mL; the feedstocks were filtered using 0.44 μm and 0.22 μm Millex-GP syringe filters (MilliporeSigma, Burlington, MA). A volume of either 0.2 mL of Ig-rich paste solution (corresponding to 1.5 mg of IgG), 0.2 mL of cryo-poor plasma solution (1.5 mg of IgG), or 0.2 mL of cryo-rich plasma solution (1.4 mg of IgG) were loaded on the column at the flow rate of 0.02 mL/min (RT: 5 min). After washing the adsorbent with 10 CVs of binding buffer at 0.1 mL/min, the bound IgG was eluted with 20 CVs of 0.2 M acetate buffer at pH 4.0 at 0.2 mL/min and neutralized upon collection using 3 M Tris buffer at pH 8.5. The adsorbent was then regenerated with 10 CVs of 0.1 M glycine buffer at pH 2.5 at 0.2 mL/min, cleaned in place with 10 CVs of aqueous 0.1 M NaOH, and finally equilibrated with binding buffer. The collected flow-through and elution fractions were analyzed by Protein G Sepharose™ Fast Flow resin to obtain IgG yield, and via size exclusion chromatography (SEC) and SDS-PAGE under reducing condition to obtain IgG purity.

Capture of non-Ig plasma proteins in flow-through mode by first- and second-generation LigaGuard™ adsorbents. The following mobile phases were prepared: 20 mM piperazine HCl buffer at pH 5.0 and 5.5; 20 mM Bis-Tris HCl buffer at pH 5.5, 6.0, 6.5, and 7.0; 20 mM citric acid and Na₂HPO₄ at pH 6.0, 6.5, 7.0, and 7.4; 20 mM KH₂PO₄ and Na₂HPO₄ at pH 6.0, 6.5, 7.0, and 7.4; 20 mM Tris HCl buffer at pH 7.0 and 7.4; and PBS buffer at pH 7.4. A volume of 0.5 mL of either first- or second-generation LigaGuard™ resin was wet packed in a 0.5 mL Alltech PEEK column, washed with 20% v/v ethanol (10 CVs) and deionized water (3 CVs), and finally equilibrated with binding buffer (10 CVs) at the flow rate of 0.5 mL/min. Pure IgG solutions were prepared by dissolving human polyclonal IgG in the above-listed buffers at 2.5 mg/mL. The Ig-depleted plasma samples were prepared as the flow-through fractions obtained by injecting 1.0 mL of cryo-rich plasma diluted 10-fold with the corresponding buffer in the columns packed with 1.0 mL HiTrap™ Protein A HP and 1.0 mL of HiTrap™ Protein G HP. A volume of 7 mL volume of either pure IgG solution or Ig-depleted diluted plasma (protein titer ˜5.0 mg/mL) was continuously loaded on the LigaGuard™ column at the flow rate of 0.5 mL/min (RT: 1 min) and the flow-through fractions were collected at 0.5 mL increments; after loading, the column was washed with 20 CVs of equilibration buffer, and a pooled wash fraction was collected until the 280 nm absorbance decreased below 50 mAU. The resin was discarded after one use (i.e., no elution or regeneration was performed). The collected fractions were analyzed by Pierce™ BCA Protein Assay Kit, Protein G Sepharose™ Fast Flow resin, size exclusion chromatography (SEC), and SDS-PAGE under reducing condition to obtain the breakthrough ratio (Equation 1), yield (Equation 2), and binding (mg of protein per mL resin, Equation 3).

$$C / {C}_{{0}_{fractional , x}} = \frac{{C}_{IgG , x}}{{C}_{I gG , L}} \qquad Equation 1$$

$${Y ( \% )}_{p o o l e d , x} = \frac{{\sum }_{x = 1}^{N} {C}_{IgG , x} {Vv}_{x}}{{C}_{I gG , L} \times {V}_{L}} \times 100 \% \qquad Equation 2$$

$${Q ( mg / mL resin )}_{pooled , x} = \frac{{\sum }_{x = 1}^{N} ( {C}_{I gG , L} - {C}_{IgG , x} ) \times {V}_{x}}{{V}_{R}} \qquad Equation 3$$

Wherein C/C₀ (%)_{fractional,x} is the fractional IgG breakthrough ratio at fraction x, Y (%)_{pooled,x} is the pooled IgG yield at fraction x, Q (mg/mL resin)_{pooled,x} is the pooled binding capacity in at fraction x, C_{IgG,x} is the IgG concentration in fraction x, Vₓ is the volume of fraction X, C_{IgG,L} is the IgG concentration in the load samples, and V_{L} is the cumulative feed volume loaded, and N is the number of fractions generated by loading V_{L}, and V_{R} is the volume of selected resin.

Purification of IgG from cryo-rich and cryo-poor human plasma using a 2-step chromatographic process comprising LigaGuard™ and LigaTrap™ resins. LigaGuard™ and LigaTrap™ resin were wet packed in 0.5 mL Alltech PEEK columns, and washed with 20% v/v ethanol (10 CVs) and deionized water (3 CVs). The LigaGuard™ resin were equilibrated with 10 CVs of 20 mM Bis-Tris HCl buffer either at pH 6.0 or 5.5 (Buffer A), while the LigaTrap™ resin was equilibrated with 10 CVs of 0.1 M phosphate buffer at pH 7.4 containing 0.5 M NaCl and 25 mM NaCapr (Buffer B). Diluted plasma, prepared as described in above, was loaded on the LigaGuard™ column at the flow rate of 0.5 mL/min (RT: 1 min), and the flow-through fractions were collected at 0.5 mL increments; the loading was chased with 20 CVs of 0.2M acetate buffer at pH 5.0 (Buffer C). The IgG-rich effluent collected during loading and buffer chasing was continuously mixed with Buffer B and injected on the LigaTrap™ column. Column loading and washing, IgG elution, and column regeneration and cleaning were performed as detailed further herein. The collected flow-through and elution fractions were analyzed by Protein G Sepharose™ Fast Flow resin to obtain IgG yield, and via size exclusion chromatography (SEC) and SDS-PAGE under reducing condition to obtain IgG purity.

Quantification of IgG yield by analytical Protein G chromatography. The IgG concentration in the collected fractions were determined by analytical Protein G chromatography using a Waters Alliance 2690 separations module system with a Waters 2487 dual absorbance detector (Waters Corporation, Milford, MA, USA). Protein G Sepharose™ Fast Flow resin wet packed in a Vici Jour PEEK 2.1 mm ID×30 mm column (0.1 mL) was equilibrated with PBS, pH 7.4. A volume of 50 μL for each sample or standard was injected, and the analytical method proceeded as outlined in Table 8. The effluent was monitored by 280 nm absorbance (A280), and the concentration was determined based on the peak area of the A280 elution peak. Pure IgG at 0, 0.1, 0.5, 1.0, 2.0, 4.0, 6.0, 8.0, and 10.0 mg/mL was utilized to construct the standard curve.

TABLE 8
HPLC method for IgG quantification by
analytical Protein G chromatography.

| Time | Flowrate | PBS, pH | 0.1M Glycine |
|---|---|---|---|
| (min) | (mL/min) | 7.4 | buffer, pH 2.5 |
| 0.00 | 0.5 | 100% | 0% |
| 2.00 | 0.5 | 100% | 0% |
| 2.01 | 0.5 | 0% | 100% |
| 6.00 | 0.5 | 0% | 100% |
| 6.01 | 0.5 | 100% | 0% |
| 10.00 | 0.5 | 100% | 0% |

To assess the breakthrough ratio (C/C₀), yield (Y, %) and binding capacity (Q, mg/mL resin) of IgG product, the values of fractional breakthrough ratio, pooled yield, and protein binding as a function of CV were calculated using Equations 1-3, respectively.

Quantification of IgG purity by size-exclusion chromatography (SEC). The collected fractions were then analyzed by analytical SEC using a Yarra 3 μm SEC-2000 300 mm×7.8 mm column operated with a 40-min isocratic method using PBS at pH 7.4 as mobile phase. A volume of 50 μL of sample was injected and the effluent continuously monitored by UV spectrometry at 280 nm absorbance (A280). The fractional purity of IgG (P, %) was calculated using Equation 4.

$${P ( \% )}_{f , x} = \frac{{A}_{IgG , x}}{{A}_{IgG , x} + {A}_{n o n - IgG PPs , x}} \times 100 \% \qquad Equation 4$$

Wherein P (%)_{f,x} is the fractional IgG purity in the x^{th} flow-through fraction, and A_{IgG,x} and A_{non-IgG PPs,x} are the values of area of the peaks respectively related to IgG and non-IgG plasma proteins (based on the residence time of the peak) in the x^{th} flow-through fraction. The derivation of Equation 4 is provided below.

Quantification of IgG purity by sodium dodecyl sulfate polyacrylamide gel electrophoresis (SDS-PAGE). The collected fractions were analyzed by SDS-PAGE using 4-20% Mini-PROTEAN™ TGX™ Precast protein gels with Tris/Glycine/SDS buffer as running buffer. The fractions were diluted or concentrated to a total protein concentration of ˜1 mg/mL and a volume of 10 μL of different samples were loaded to the wells of SDS-PAGE gels. The sample stripes were concentrated under 80 V for about 30 min and separated under 120 V for about 1 h. Then the gels were stained by Coomassie Brilliant Blue R-250 Staining solution for about 25 min and then distained using 10% glacial acetic acid, 5% ethanol dissolved in Milli-Q water. Finally, the stained protein strips were imagined by the Gel Doc2000 imaging system from Bio-Rad.

Derivation of Equation 4. The purity of human IgG contained in the x^{th} flow-through fraction is rigorously defined by Equation 4a, wherein C_{IgG,x} and C_{non-IgG PPs,x} are respectively the concentrations of IgG and non-IgG plasma proteins in the x^{th} fraction:

$${P ( \% )}_{f , x} = \frac{{C}_{IgG , x}}{{C}_{IgG , x} + {C}_{n o n - IgG PPs , x}} \times 100 \% \qquad Equation 4 a$$

In a SEC chromatogram, the area of the peak corresponding to human IgG and the total area of the peaks corresponding to non-IgG plasma proteins—identified based on residence time—are directly proportional to the concentration of IgG and non-IgG plasma proteins. The Lambert-Beer law states, in fact, that the concentration of a protein in solution is equal to the product of the molar extinction coefficient of the protein (2) times the light absorbance of the solution (A). Applying the Lambert-Beer law to Equation 4a returns Equation 4b:

$${P ( \% )}_{f , x} = \frac{{λ}_{IgG , x} {A}_{IgG , x}}{{λ}_{IgG , x} {A}_{IgG , x} + {λ}_{non - IgG PPs , X} {A}_{non - IgG PPs , x}} \times 100 \% \qquad Equation 4 b$$

Upon assuming that the molar extinction coefficients of all plasma proteins are similar (λ_{IgG}˜λ_{non-IgG PPs}), Equation 4b ultimately yields Equation 4:

$${P ( \% )}_{f , x} = \frac{{A}_{IgG , x}}{{A}_{IgG , x} + {A}_{n o n - IgG PPs , x}} \times 100 \% \qquad Equation 4$$

It is noted that the assumption λ_{IgG}˜λ_{non-IgG} plasma proteins is an approximation. However, it is commonly utilized in bioprocess analytics to evaluate IgG purity.

1. A composition for purifying a target biologic from a biological fluid, wherein the composition comprises at least one peptide ligand that is at least four amino acids in length and comprises at least one basic amino acid and at least one hydrophilic amino acid.

2. The composition of claim 1, wherein the at least one peptide ligand comprises a hydrophobic amino acid or a positively charged amino acid at the second amino acid position.

3. The composition of claim 1, wherein the at least one peptide ligand comprises a hydrophobic amino acid or a negatively charged amino acid at the fourth amino acid position.

4. The composition of claim 1, wherein the at least one peptide ligand comprises an acidic amino acid directly adjacent to a hydrophobic amino acid or a negatively charged amino acid.

5. The composition of claim 1, wherein the at least one peptide ligand comprises a polar amino acid directly adjacent to cationic amino acid or an aliphatic amino acid.

6. The composition of claim 1, wherein a negatively charged amino acid in the peptide ligand is: (i) not directly adjacent to a polar amino acid; (ii) directly adjacent to an aliphatic amino acid or an aromatic amino acid; and/or (iii) directly adjacent to a positively charged amino acid.

7. The composition of claim 1, wherein an aromatic amino acid in the peptide ligand is: (i) directly adjacent to an aliphatic amino acid; (ii) directly adjacent to an anionic amino acid; and/or (iii) directly adjacent to a cationic amino acid.

8. The composition of claim 1, wherein the at least one peptide ligand binds at least one host cell protein (HCP), at least one high-risk HCP, at least one host cell nucleic acid, aggregates of the target biologic, and/or an impurity derived from the target biologic.

9. The composition of claim 8, wherein the at least one peptide ligand exhibits a K_{D} from about 10⁻⁹ M to about 10⁻⁵ M for the HCP, the host cell nucleic acid, the aggregates of the target biologic, and/or the impurity derived from the target biologic.

10. The composition of claim 1, wherein the at least one peptide ligand comprises a linker.

11. The composition of claim 10, wherein the linker is bound to the C-terminus of the peptide ligand, and wherein the linker comprises a Glyₙ or a [Gly-Ser-Gly]ₘ, wherein 6≥n≥1 and 3≥m≥1.

12. The composition of claim 1, wherein the at least one peptide ligand is bound to a solid support.

13-14. (canceled)

15. The composition of claim 1, wherein the at least one peptide ligand is no more than 15 amino acids in length.

16. The composition of claim 1, wherein the biological fluid comprises a supernatant and/or a cellular lysate.

17-25. (canceled)

26. The composition of claim 1, wherein the target biologic is one or more of a protein, peptide or polypeptide; an oligonucleotide or a polynucleotide; a virus or a virus-like particle; an exosome or an extracellular vesicle; a cell or cell organelle; or a small molecule.

27. The composition of claim 1, wherein the biological fluid comprises a pH from about 3.0 to about 9.0 and/or comprises a conductivity of about 1 to about 50 mS/cm.

28. (canceled)

29. The composition of claim 1, wherein the at least one peptide ligand is selected from the group consisting of: EHIPA (SEQ ID NO: 1), GPRPK (SEQ ID NO: 2), HAIYPHRH (SEQ ID NO: 3), DLSLRDWGCLW (SEQ ID NO: 4), and DISLPRWGCLW (SEQ ID NO: 5), or any derivatives or variants thereof.

30-33. (canceled)

34. The composition of claim 29, wherein the composition further comprises at least one peptide ligand selected from the group consisting of: GSRYRY (SEQ ID NO: 6), RYYYAI (SEQ ID NO: 7), AAHIYY (SEQ ID NO: 8), IYRIGR (SEQ ID NO: 9), HSKIYK (SEQ ID NO: 10), ADRYGH (SEQ ID NO: 11), DRIYYY (SEQ ID NO: 12), DKQRII (SEQ ID NO: 13), RYYDYG (SEQ ID NO: 14), YRIDRY (SEQ ID NO: 15), HYAI (SEQ ID NO: 16), FRYY (SEQ ID NO: 17), HRRY (SEQ ID NO: 18), RYFF (SEQ ID NO: 19), DKSI (SEQ ID NO: 20), DRNI (SEQ ID NO: 21), HYFD (SEQ ID NO: 22), and YRFD (SEQ ID NO: 23), or any derivatives or variants thereof.

35. An adsorbent comprising the composition of claim 1.

36. A method of purifying a target biologic from a biological fluid, the method comprising:
contacting the composition comprising the at least one peptide ligand of claim 1, or the adsorbent of claim 35, with a biological fluid comprising a target biologic; and
collecting the biological fluid in flow-through mode, wherein the biological fluid comprises the target biologic;
wherein the at least one peptide ligand binds at least one host cell protein (HCP), at least one high-risk HCP, at least one host cell nucleic acid, aggregates of the target biologic, and/or an impurity derived from the target biologic in a retentate.

37-41. (canceled)
